(19) United States
(12) Patent Application Publication
(10) Pub. No.: US 2018/0110831 A1
(43) Pub. Date: Apr. 26, 2018

(54) CD 47 CONTAINING POROUS NANOPARTICLE SUPPORTED LIPID BILAYERS (PROTOCELLS) FIELD OF THE INVENTION

(71) Applicant: STC.UNM, Albuquerque, NM (US)

(72) Inventors: C. Jeffrey Brinker, Albuquerque, NM (US); Eric C. Carnes, Albuquerque, NM (US); Carlee Erin Ashley, Albuquerque, NM (US); Jacob Ongundi Agola, Albuquerque, NM (US); Kimberly Butler, Albuquerque, NM (US); Christophe Theron, Villeneuve les Maguelone (FR)

(21) Appl. No.: 15/557,000

(22) PCT Filed: Mar. 9, 2016
(86) PCT No.: PCT/US2016/021490

§ 371 Date: Sep. 8, 2017

Related U.S. Application Data
(60) Provisional application No. 62/130,392, filed on Mar. 9, 2015.

(51) Int. Cl. A61K 38/17; A61K 47/64; A61K 47/69; A61K 9/51; C07K 14/705

(52) U.S. Cl. CPC A61K 38/1774; A61K 47/6425; A61K 9/127; A61K 9/5123; C07K 14/70546; A61K 47/6911

(57) ABSTRACT

The present invention is directed to protocells, which have a core and a lipid bilayer surrounding the core, with at least one CD47 molecule or an active fragment thereof in or conjugated to the lipid bilayer. The CD47 present on the lipid bilayer allows the protocell to evade phagocytosis by macrophages, and can be conjugated to the lipid bilayer via a crosslinker. The protocell can be loaded with a diagnostic or therapeutic cargo, such as a polypeptide, a nucleic acid, or a drug. The protocell can also include a targeting species for targeted delivery of the cargo to a cell. The protocell can also include an endosomolytic peptide, which promotes endosomal escape after uptake by the targeted cell. The protocells with CD47 on the lipid bilayer provide better circulation after in vivo administration compared to protocells without CD47, and are therefore particularly useful as a cargo delivery vehicle.

### CLAIM OF PRIORITY

This application claims priority benefit under 35 U.S.C. § 119(e) to provisional U.S. Patent Application 62/130,392, filed on Mar. 9, 2015, the entire contents of which are hereby incorporated by reference.

### GOVERNMENT SUPPORT

This invention was made with government support under DE-AC04-94AL85000 awarded by the United States Department of Energy. The government has certain rights in the invention.

### FIELD OF THE INVENTION

This invention generally relates to nanoparticles coated with a lipid bilayer, which includes components to evade macrophage phagocytosis or enhance biodistribution and/or circulation.

### BACKGROUND OF THE INVENTION

Engineered nanoparticle-cellular interactions encompass a broad spectrum of emerging topics in National Security and the improved Health and Well Being of the Nation. Engineered nanoparticles promise to enable detection of diseases caused by biological or chemical agents, as well as selective delivery of customized ‘cocktails’ of diagnostic and therapeutic agents to diseased cells or organs. Understanding and engineering nanoparticle-cellular interactions at scales that span the sub-cellular to whole organism levels are critical to the effective design of nanoparticle delivery vehicles (or ‘nanocarriers’) that increase the efficacy and safety of existing and novel therapeutics. Although hundreds of engineered nanocarriers are under development, they all fall short of addressing the multiple challenges of targeted delivery, which prevents them from being systematically engineered to establish structure-function relationships or to address personalized medicine.

To this end, we recently invented a new composite nanocarrier (the ‘protocell’), which was originally reported in a 2011 cover article in Nature Materials (Nat Mater 2011, 10, 389-397) and highlighted in an accompanying News & Views commentary, the author of which stated, ‘The properties engineered into this [protocell] system elegantly synergize to approach the goal of an ideal targeted-delivery agent.’ Protocells are formed via fusion of lipid bilayers (similar in composition to cell membranes) onto nanoporous, cargo-loaded nanoparticle cores; the lipid shell of the protocell can then be modified with targeting and trafficking ligands to tailor their in vivo behavior according to the disease of interest. Protocells combine advantages of FDA-approved liposomes (low inherent toxicity and immunogenicity; long circulation times) and porous nanoparticles (enormous capacities for multiple physicochemically disparate cargos; high degree of chemical and colloidal stability). In addition, the protocell's supported lipid bilayer (SLB) is a reconfigurable surface that can engage in complex biomolecular interactions with target cells, tissues, or organs in order to avoid off-target binding and maximize on-target internalization and intracellular trafficking. This invention exploits the modular nature and synergistic properties of the protocell in order to design and fabricate next-generation protocells, which will have tailorable circulation times, rapid accumulation at target sites, controllable release rates, and reproducible degradation and excretion.

### BRIEF DESCRIPTION OF THE INVENTION

Provided herein is a protocell comprising a nanoparticulate core surrounded by a lipid bilayer, wherein the lipid bilayer comprises a CD47 molecule or an active fragment thereof conjugated to the lipid bilayer. Also provided are pharmaceutical compositions comprising the protocell; a method of treating cancer in a subject, comprising administering to the subject the pharmaceutical composition, wherein the protocells are loaded with an anticancer agent; a method of treating a viral infection in a subject comprising administering to the subject the pharmaceutical composition, wherein the protocells are loaded with an antiviral agent; a method of delivering a cargo to a cell comprising contacting the protocell with a cell, such as a cancer cell; a method of enhancing the in vivo circulation of a protocell in a subject, comprising administering the protocell to the subject; and a method of making the protocell comprising conjugating a CD47 molecule or an active fragment thereof to the lipid bilayer, wherein the lipid bilayer surrounds the core. In any of the described embodiments, the CD47 molecule or active fragment thereof can be recombinantly produced. The CD47 molecule fragment can be a CD47 extracellular domain.

The CD47 molecule or an active fragment thereof can be conjugated to the lipid bilayer via a linker, such as a chemical linker. The linker can be, for example, a heterobifunctional crosslinker, such as an amine-to-carboxylic acid crosslinker or an amine-to-sulfhydryl crosslinker. The crosslinker can be formed using thyl(dimethylaminopropyl) carbodiimide and N-hydroxylsuflosuccinimide. In some embodiments, the heterobifunctional crosslinker comprises a maleimide reactive group and an N-hydroxysuccinimide ester reactive group, such as SM-PEGₙ. In some embodiments, the crosslinker is propargyl-PEG-maleimide.

The CD47 molecule or the active fragment thereof can be conjugated to a lipid comprising a primary amine or an azide moiety. In another embodiment, the CD47 molecule or the active fragment thereof can be conjugated to the lipid bilayer via chelation, for example by conjugating to a lipid comprising a divalent cation and a nitrilotriacetic acid moiety or iminodiacetic acid moiety. In some embodiments, the CD47 molecule or active fragment thereof comprises a polyhistidine tag, which can be on the N-terminus or the C-terminus of the CD47 molecule or active fragment thereof.

In any of the embodiments described above, the protocell comprises an effective amount of CD47 molecules conjugated to the lipid bilayer that allows the protocell to avoid macrophage phagocytosis. For example, in some embodiments, the protocell has about 21 or more copies of the CD47 molecule or active fragment thereof.

Optionally, the lipid bilayer of the protocell further comprises a cell-targeting species (such as a cell-targeting peptide) or a fusogenic peptide.

In some embodiments, the protocell is loaded with a cargo, such as a diagnostic agent or a therapeutic agent. The cargo can be selected from the group consisting of a nucleic acid, a polypeptide, a drug, and an imaging agent. In some embodiments, the cargo is DNA, such as a plasmid DNA or a double stranded linear DNA. The DNA can encode a polypeptide toxin, a reporter protein, shRNA, or siRNA. In some embodiments, the cargo is RNA, such as siRNA, shRNA, miRNA, or antisense RNA. In some embodiments, the cargo is conjugated to a nuclear localization sequence. The drug can be an anticancer agent or an antiviral agent.

The core of the protocell can be porous. In some embodiments, the core comprises silica or a metal oxide. The core of the protocell can have a diameter of about 10 nm to about 250 nm, such as about 30 nm to about 100 nm. The protocell can have a diameter of about 10 nm to about 250 nm, such as about 30 nm to about 100 nm.

In some embodiments, the lipid bilayer comprises a lipid selected from the group consisting of 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-dioleoyl-sn-glycero-3-[phosphor-L-serine] (DOPS), 1,2-dioleoyl-3-trimethylammonium-propane (18:1 DOTAP), 1,2-dioleoyl-sn-glycero-3-phospho-(1′-rac-glycerol) (DOPG), 1,2-dioleoyl-sn-glycero-3-phosphoethanolamine (DOPE), 1,2-dipalmitoyl-sn-glycero-3-phosphoethanolamine (DPPE), 1,2-dioleoyl-sn-glycero-3-phosphoethanolamine-N-[methoxy(polyethylene glycol)-2000] (18:1 PEG-2000 PE), 1,2-dipalmitoyl-sn-glycero-3-phosphoethanolamine-N-[methoxy(polyethylene glycol)-2000] (16:0 PEG-2000 PE), 1-Oleoyl-2-[12-[(7-nitro-2-1,3-benzoxadiazol-4-yl)amino]lauroyl]-sn-Glycero-3-Phosphocholine (18:1-12:0 NBD PC), 1-palmitoyl-2-{12-[(7-nitro-2-1,3-benzoxadiazol-4-yl)amino]lauroyl}-sn-glycero-3-phosphocholine (16:0-12:0 NBD PC), and cholesterol.

The protocell can comprise an organosilane, such as an amine-containing silane. The amine-containing silane can comprise a primary amine, a secondary amine, a tertiary amine, N-(2-aminoethyl)-3-aminopropyltrimethoxysilane (AEPTMS), 3-aminopropyltrimethoxysilane (APTMS), (5) 3-aminopropyltriethoxysilane (APTS), an amino-functional trialkoxysilane, a protonated secondary amine, a protonated tertiary alkyl amine, a protonated amidine, a protonated guanidine, a protonated pyridine, a protonated pyrimidine, a protonated pyrazine, a protonated purine, a protonated imidazole, a protonated pyrrole, a quaternary alkyl amines, or a combinations thereof.

The core can have a BET surface area greater than about 600 m²/g. The core can also have a pore volume fraction of between about 60% to about 70 and/or a multimodal pore morphology In some embodiments, the core comprises pores having an average diameter of between about 20 nm to about 30 nm. In some embodiments, the core has surface-accessible pores interconnected by pores having an average diameter of between about 5 nm to about 15 nm.

In some embodiments, the lipid bilayer comprises one or more stratum corneum permeability-enhancers selected from the group consisting of a monounsaturated omega-9 fatty acid, an alcohol, a diol, a solvent, a co-solvent, R8 peptide, and an edge activator. The monounsaturated omega-9 fatty acid can be selected from the group consisting of oleic acid, elaidic acid, eicosenoic acid, mead acid, erucic acid, and nervonic acid; the alcohol can be selected from the group consisting of methanol, ethanol, propanol, and butanol; the solvent and co-solvent can be selected from the group consisting of PEG 400 and DMSO; the diol can be selected from the group consisting of ethylene glycol and polyethylene glycol; and the edge activator can be selected from the group consisting of bile salts, polyoxyethylene esters and polyoxyethylene ethers, and a single-chain surfactant, and mixtures thereof.

Also provided is a protocell composition comprising a plurality of the protocells described herein. The plurality of protocells can be monodisperse, for example having a polydispersity index of about 0.1 or less. The average diameter of the protocells can be about 10 nm to about 250 nm, for example about 30 nm to about 100 nm.

Further described is a pharmaceutical composition comprising the protocell composition described herein and a pharmaceutically acceptable excipient. The pharmaceutical composition can further comprise a drug which is not disposed within the protocell. The drug can be an antiviral agent or an anticancer agent. The pharmaceutical composition can be in parenteral dosage form, topical dosage form, or transdermal dosage form. For example, the pharmaceutical composition can be in an intradermal, intramuscular, intraosseous, intraperitoneal, intravenous, subcutaneous, or intrathecal dosage form.

Certain embodiments of the present invention are directed to protocells for specific targeting of cells, in particular aspects, cancer cells and infected cells (e.g., bacteria and virus infections).

Applicants incorporate by reference in its entirety, international application PCT/US2012/060072, filed 12 Oct. 2012 and published as WO 2013/056132 18 Apr. 2013.

### BRIEF DESCRIPTION OF THE FIGURES

FIG. 1 shows that the nanoparticles according to one embodiment used in the present invention which are prepared by an aerosol-assisted EISA process can be altered to control particle size and distribution.

FIG. 2A shows the pore size and framework designed to be tailorable for multiple types of cargo and that aerosolized auxiliary components are easily incorporated according to one embodiment.

FIG. 2B shows that that a, b c, and e of FIG. 2A are templated by CTAB, B58, P123 and PS+B56. A, B, C, D and E are templated by CTAP+NaCl, 3% wt P123, 3% wt P123+poly(propylene glycol acrylate), microemulsion and CTAB(NH₄)₂SO₄.

FIG. 3 shows that pore surface chemistry (i.e., charge and hydrophobicity) and pore size is controlled principally by co-condensation of organo-silanes and silicic acids either by co-self-assembly or post-self-assembly derivatization according to one embodiment. See Lin, et al., Chem. Mater. 15, 4247-56 2003; Liu, J. et al., J. Phys. Chem., 104, 8328-2339, 2000; Fan, H. et al., Nature, 405, 56-60, 2000 and Lu, Y. et al., J Am. Chem. Soc., 122, 5258-5261, 2000.

FIG. 4 shows a protocell with a core surrounded by a lipid bilayer. CD47, targeting peptides, or endosomolytic peptides can be conjugated to the lipid bilayer to the lipid bilayer using a crosslinker, for example the crosslinker illustrated in FIG. 4.

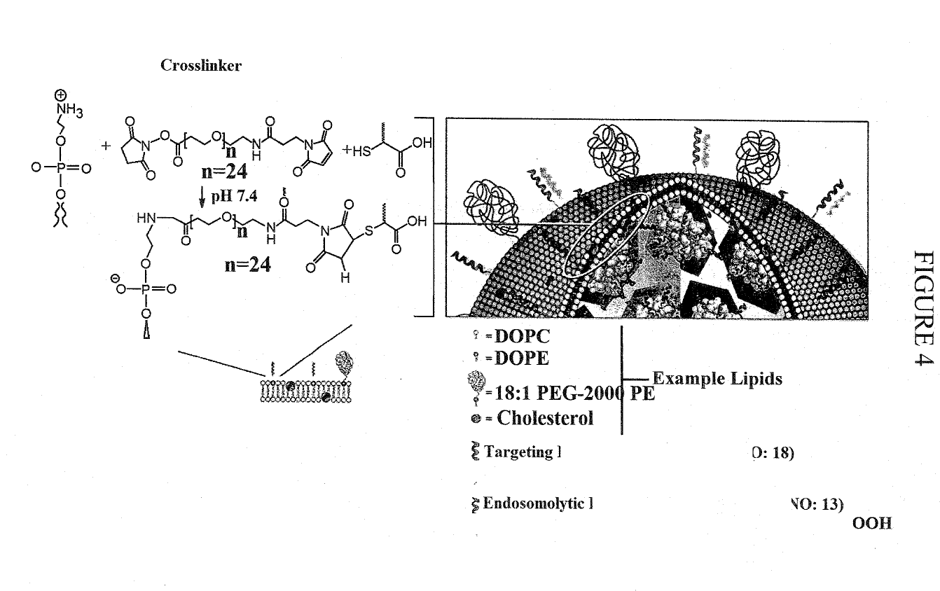

FIG. 5 illustrates chemical structure of various crosslinkers that can be utilized to attach CD47 or other polypeptides to the lipid bilayer of the protocell.

FIG. 6 illustrate exemplary lipids that could be present in the lipid bilayer. For example, the lipid bilayer can comprise DSPC, cholesterol, DSPE-PEG, or any other lipid disclosed herein. Functionalized lipids, such as the ones illustrated in FIG. 6, are useful to conjugate CD47 or other polypeptides to the lipid bilayer, as described herein.

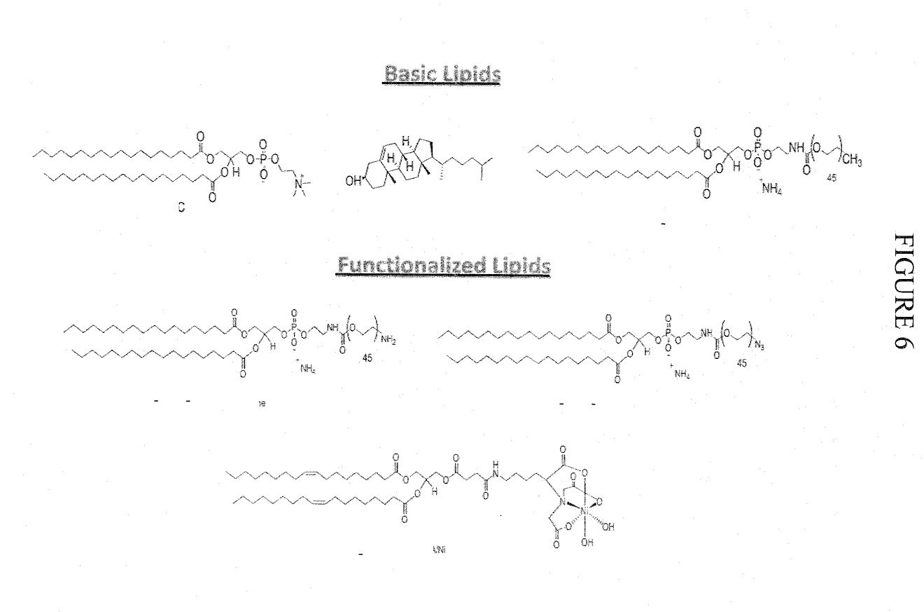

FIG. 7 presents fluorescence microscopy images of macrophages after incubation with protocells conjugated to CD47 using EDC/Sulfo-NHS crosslinkers for 30 minutes, 2 hours, 4 hours, 8 hours, or 24 hours. The protocell cores were fluorescently labeled, the cytoskeleton was dyed using phalloidin, and the nuclei were stained using Hoescht stain.

FIG. 8 presents fluorescence microscopy images of macrophages after incubation with protocells conjugated to CD47 using a SM-PEG₁₂ crosslinker for 30 minutes, 2 hours, 4 hours, 8 hours, or 24 hours. The protocell cores were fluorescently labeled, the cytoskeleton was dyed using phalloidin, and the nuclei were stained using Hoescht stain.

FIG. 9 presents fluorescence microscopy images of macrophages after incubation with protocells conjugated to CD47 using click chemistry linker Propargyl-PEG-maleimide for 30 minutes, 2 hours, 4 hours, 8 hours, or 24 hours. The protocell cores were fluorescently labeled, the cytoskeleton was dyed using phalloidin, and the nuclei were stained using Hoescht stain.

FIG. 10 presents fluorescence microscopy images of macrophages after incubation with protocells conjugated to CD47 using Ni/NTA functionalized lipid and His-tagged CD47 for 30 minutes, 2 hours, 4 hours, 8 hours, or 24 hours. The protocell cores were fluorescently labeled, the cytoskeleton was dyed using phalloidin, and the nuclei were stained using Hoescht stain.

FIG. 11 presents fluorescence microscopy images of macrophages after incubation with mesoporous silica nanoparticles for 30 minutes, 2 hours, 4 hours, 8 hours, or 24 hours. The nanoparticles were fluorescently labeled, the cytoskeleton was dyed using phalloidin, and the nuclei were stained using Hoescht stain.

FIG. 12 presents fluorescence microscopy images of macrophages after incubation with protocells conjugated to PEG for 30 minutes, 2 hours, 4 hours, 8 hours, or 24 hours. The protocell cores were fluorescently labeled, the cytoskeleton was dyed using phalloidin, and the nuclei were stained using Hoescht stain.

FIG. 13 presents fluorescence microscopy images of macrophages after incubation with protocells conjugated to CD47 using various crosslinking techniques and pre-incubated with anti-CD47 antibodies. The protocell cores were fluorescently labeled, the cytoskeleton was dyed using phalloidin, and the nuclei were stained using Hoescht stain.

FIG. 14 presents mean fluorescence intensity of macrophages incubated with fluorescently labeled mesoporous silica nanoparticles, protocells conjugated to CD47 by various crosslinkers, and protocells conjugated to CD47 by various crosslinkers and pre-incubated with anti-CD47 antibody. As a control, the mean fluorescence intensity was measured for macrophages not incubated with particles.

FIG. 15 presents fluorescence microscopy images of macrophages after incubation with protocells conjugated to CD47 using Ni/NTA functionalized lipid and various amounts of His-tagged CD47. The numbers indicate the concentration of CD47 incubated with the protocells when conjugating the CD47 to the lipid bilayer. The density of CD47 on the lipid bilayer can be converted to approximate number of CD47 molecules, as reflected in Table 2. The protocell cores were fluorescently labeled, the cytoskeleton was dyed using phalloidin, and the nuclei were stained using Hoescht stain.

### DETAILED DESCRIPTION OF THE INVENTION

Provided herein is a protocell (i.e., a lipid coated nanoparticle), which comprises a nanoparticle core surrounded by a lipid bilayer, wherein a CD47 molecule or active fragment thereof is conjugated to the lipid bilayer. The CD47 molecule or active fragment thereof can be conjugated to the lipid bilayer, for example, via a linker, such as a chemical linker. In some embodiments, the CD47 molecule fragment is a CD47 extracellular domain. The lipid bilayer of the protocell can be a synthetic lipid bilayer (i.e., not formed from a cellular plasma membrane). The CD47 molecule or active fragment thereof conjugated to the lipid bilayer can be recombinantly produced. The core can be a porous core, which may comprise silica. In some embodiments, the lipid bilayer is a supported lipid bilayer. In some embodiments, the protocell comprises an effective number of copies of the CD47 molecule or the active fragment thereof to avoid macrophage phagocytosis. For example, the protocell can comprise about 21 or more copies of the CD47 molecule or active fragment thereof. In some embodiments, the protocell further comprises a targeting species and/or a fusogenic peptide. The protocell can also be loaded with a cargo (such as a therapeutic agent or a diagnostic agent), which can be delivered to a cell, such as a cancer cell. The protocells can have a diameter of about 10 nm to about 250 nm, such as 30 nm to about 100 nm. Also provided herein is a protocell composition comprising a plurality of the protocells. In some embodiments, the plurality of protocells is monodisperse, for example can have a polydispersity index of about 0.1 or less. The average diameter of the protocells can be between about 10 nm and about 250 nm, such as between 30 nm and about 100 nm. The protocell composition can be included in a pharmaceutical composition, which further comprises a pharmaceutically acceptable excipient. The pharmaceutical composition can be used to treat cancer in a subject when the protocells are loaded with an anticancer agent. The pharmaceutical composition can also be used to treat a viral infection in a subject when the protocells are loaded with an antiviral agent.

In some embodiments, there is provided a protocell comprising a nanoparticulate core surrounded by a supported lipid bilayer, wherein the lipid bilayer comprises a CD47 molecule or active fragment thereof conjugated to the lipid bilayer. Optionally, the CD47 molecule or active fragment thereof is recombinantly produced. In some embodiments, the CD47 molecule fragment is a CD47 extracellular domain. Also optionally, the lipid bilayer is a synthetic lipid bilayer. In some embodiments, the protocell comprises an effective number of copies of the CD47 molecule or the active fragment thereof to avoid macrophage phagocytosis. For example, the protocell can comprise about 21 or more copies of the CD47 molecule or active fragment thereof. In some embodiments, the protocell further comprises a targeting species and/or a fusogenic peptide. In some embodiments, the protocell comprises a cargo, for example a diagnostic agent or a therapeutic agent. The cargo can be a nucleic acid, a polypeptide, a drug, or an imaging agent. A plurality of the protocells can be included in a protocell composition. In some embodiments, the plurality of protocells is monodisperse, for example can have a polydispersity index of about 0.1 or less. In some embodiments, the protocells in the protocell composition have an average diameter of about 10 nm to about 250 nm, such as about 30 nm to about 100 nm. The protocell composition can be included in a pharmaceutical composition, which further comprises a pharmaceutically acceptable excipient. The pharmaceutical composition can be used to treat cancer in a subject when the protocells are loaded with an anticancer agent. The pharmaceutical composition can also be used to treat a viral infection in a subject when the protocells are loaded with an antiviral agent. The protocell can be used to deliver a cargo to a cell, such as a cancer cell or virally-infected cell. The protocell comprising the CD47 molecule or fragment thereof can also be administered to a subject to enhance in vivo circulation of the protocell. The CD47 molecule or fragment thereof can also be conjugated to the lipid bilayer of the protocell to decrease phagocytosis of the protocell by macrophages.

In some embodiments, there is provided a protocell comprising a nanoparticulate core surrounded by a lipid bilayer, wherein the lipid bilayer comprises a CD47 molecule or active fragment thereof conjugated to the lipid bilayer via a chemical linker (such as an amine-to carboxylic acid crosslinker or an amine-to-sulfhydryl crosslinker). In one example, the chemical crosslinker comprises a malemide reactive group and an N-hydroxysuccinimide ester reactive group (for example SM-PEGₙ, where n indicates the number of PEG subunits in the crosslnker). In some embodiments, the CD47 molecule fragment is a CD47 extracellular domain. In some embodiments, the protocell comprises an effective number of copies of the CD47 molecule or the active fragment thereof to avoid macrophage phagocytosis. For example, the protocell can comprise about 21 or more copies of the CD47 molecule or active fragment thereof. In some embodiments, the protocell further comprises a targeting species and/or a fusogenic peptide. The protocell can have a diameter between about 10 nm and about 250 nm, such as between about 30 nm and about 100 nm. In some embodiments, the protocell comprises a cargo, for example a diagnostic agent or a therapeutic agent. The cargo can be a nucleic acid, a polypeptide, a drug, or an imaging agent. A plurality of the protocells can be included in a protocell composition. In some embodiments, the plurality of protocells is monodisperse, for example can have a polydispersity index of about 0.1 or less. In some embodiments, the protocells in the protocell composition have an average diameter of about 10 nm to about 250 nm, such as about 30 nm to about 100 nm. The protocell composition can be included in a pharmaceutical composition, which further comprises a pharmaceutically acceptable excipient. The pharmaceutical composition can be used to treat cancer in a subject when the protocells are loaded with an anticancer agent. The pharmaceutical composition can also be used to treat a viral infection in a subject when the protocells are loaded with an antiviral agent. The protocell can be used to deliver a cargo to a cell, such as a cancer cell or virally-infected cell. The protocell comprising the CD47 molecule or fragment thereof can also be administered to a subject to enhance in vivo circulation of the protocell. The CD47 molecule or fragment thereof can also be conjugated to the lipid bilayer of the protocell to decrease phagocytosis of the protocell by macrophages.

In some embodiments, there is provided a protocell comprising a nanoparticulate core surrounded by a lipid bilayer, wherein a CD47 molecule or active fragment thereof is conjugated to the lipid bilayer via chelation. For example, a lipid in the lipid bilayer can comprise a divalent cation and a nitrilotriacetic acid moiety or an iminodiacetic acid moiety, and the CD47 molecule or active fragment thereof comprises a His tag. The His tag on the CD47 molecule or active fragment thereof can then coordinate with the divalent cation. In some embodiments, the CD47 fragment is a CD47 extracellular domain. In some embodiments, the protocell comprises an effective number of copies of the CD47 molecule or the active fragment thereof to avoid macrophage phagocytosis. For example, the protocell can comprise about 21 or more copies of the CD47 molecule or active fragment thereof. In some embodiments, the protocell further comprises a targeting species and/or a fusogenic peptide. The protocell can have a diameter between about 10 nm and about 250 nm, such as between about 30 nm and about 100 nm. In some embodiments, the protocell comprises a cargo, for example a diagnostic agent or a therapeutic agent. The cargo can be a nucleic acid, a polypeptide, a drug, or an imaging agent. A plurality of the protocells can be included in a protocell composition. In some embodiments, the plurality of protocells is monodisperse, for example can have a polydispersity index of about 0.1 or less. In some embodiments, the protocells in the protocell composition have an average diameter of about 10 nm to about 250 nm, such as about 30 nm to about 100 nm. The protocell composition can be included in a pharmaceutical composition, which further comprises a pharmaceutically acceptable excipient. The pharmaceutical composition can be used to treat cancer in a subject when the protocells are loaded with an anticancer agent. The pharmaceutical composition can also be used to treat a viral infection in a subject when the protocells are loaded with an antiviral agent. The protocell can be used to deliver a cargo to a cell, such as a cancer cell or virally-infected cell. The protocell comprising the CD47 molecule or fragment thereof can also be administered to a subject to enhance in vivo circulation of the protocell. The CD47 molecule or fragment thereof can also be conjugated to the lipid bilayer of the protocell to decrease phagocytosis of the protocell by macrophages.

In any of the embodiments described herein, the protocell comprises an effective number of copies of the CD47 molecule or the active fragment thereof to avoid macrophage phagocytosis. For example, the protocell can comprise about 21 or more copies of the CD47 molecule or active fragment thereof. In some embodiments, the CD47 fragment is a CD47 extracellular domain. In some embodiments, there is a protocell comprising a nanoparticulate core surrounded by a lipid bilayer, wherein an effective number of copies of a CD47 molecule or an active fragment thereof are conjugated to the lipid bilayer. In some embodiments, the protocell further comprises a targeting species and/or a fusogenic peptide. The protocell can have a diameter between about 10 nm and about 250 nm, such as between about 30 nm and about 100 nm. In some embodiments, the protocell comprises a cargo, for example a diagnostic agent or a therapeutic agent. The cargo can be a nucleic acid, a polypeptide, a drug, or an imaging agent. A plurality of the protocells can be included in a protocell composition. In some embodiments, the plurality of protocells is monodisperse, for example can have a polydispersity index of about 0.1 or less. In some embodiments, the protocells in the protocell composition have an average diameter of about 10 nm to about 250 nm, such as about 30 nm to about 100 nm. The protocell composition can be included in a pharmaceutical composition, which further comprises a pharmaceutically acceptable excipient. The pharmaceutical composition can be used to treat cancer in a subject when the protocells are loaded with an anticancer agent. The pharmaceutical composition can also be used to treat a viral infection in a subject when the protocells are loaded with an antiviral agent. The protocell can be used to deliver a cargo to a cell, such as a cancer cell or virally-infected cell. The protocell comprising the CD47 molecule or fragment thereof can also be administered to a subject to enhance in vivo circulation of the protocell. The CD47 molecule or fragment thereof can also be conjugated to the lipid bilayer of the protocell to decrease phagocytosis of the protocell by macrophages.

In any of the embodiments described herein, the protocell can comprise a cargo, for example a diagnostic agent or a therapeutic agent. In some embodiments, the cargo is a nucleic acid, a polypeptide, a drug, or an imaging agent.

A protocell comprising CD47 molecule or active fragment thereof conjugated to the lipid bilayer enhances in vivo circulation of the protocell after the protocell is administered to a subject. Accordingly, further provided herein is a method of enhancing the in vivo circulation of a protocell in a subject, comprising administering the protocell to the subject, wherein the protocell comprises a nanoparticulate core surrounded by a lipid bilayer, and a CD47 molecule or an active fragment thereof is conjugated to the lipid bilayer. In some embodiments, the CD47 fragment is a CD47 extracellular domain. In some embodiments, the protocell comprises an effective number of copies of the CD47 molecule or the active fragment thereof to avoid macrophage phagocytosis. For example, the protocell can comprise about 21 or more copies of the CD47 molecule or active fragment thereof. In some embodiments, the protocell further comprises a targeting species and/or a fusogenic peptide. The protocell can have a diameter between about 10 nm and about 250 nm, such as between about 30 nm and about 100 nm. In some embodiments, the protocell comprises a cargo, for example a diagnostic agent or a therapeutic agent. The cargo can be a nucleic acid, a polypeptide, a drug, or an imaging agent. A plurality of the protocells can be included in a protocell composition. In some embodiments, the plurality of protocells is monodisperse, for example can have a polydispersity index of about 0.1 or less. In some embodiments, the protocells in the protocell composition have an average diameter of about 10 nm to about 250 nm, such as about 30 nm to about 100 nm. The protocell composition can be included in a pharmaceutical composition, which further comprises a pharmaceutically acceptable excipient. The pharmaceutical composition can be used to treat cancer in a subject when the protocells are loaded with an anticancer agent. The pharmaceutical composition can also be used to treat a viral infection in a subject when the protocells are loaded with an antiviral agent. The protocell can be used to deliver a cargo to a cell, such as a cancer cell or virally-infected cell. The protocell comprising the CD47 molecule or fragment thereof can also be administered to a subject to enhance in vivo circulation of the protocell. The CD47 molecule or fragment thereof can also be conjugated to the lipid bilayer of the protocell to decrease phagocytosis of the protocell by macrophages.

The CD47 molecule or active fragment conjugated to the lipid bilayer of a protocell decreases phagocytosis of the protocells by macrophages. Accordingly, also provided herein is a method of decreasing uptake of a protocell by a macrophage comprising conjugating a CD47 molecule or active fragment thereof to the lipid bilayer of the protocell and exposing the protocell to an environment comprising a macrophage. In some embodiments, the CD47 fragment is a CD47 extracellular domain. In some embodiments, the protocell comprises an effective number of copies of the CD47 molecule or the active fragment thereof to avoid macrophage phagocytosis. For example, the protocell can comprise about 21 or more copies of the CD47 molecule or active fragment thereof. In some embodiments, the protocell further comprises a targeting species and/or a fusogenic peptide. The protocell can have a diameter between about 10 nm and about 250 nm, such as between about 30 nm and about 100 nm. In some embodiments, the protocell comprises a cargo, for example a diagnostic agent or a therapeutic agent. The cargo can be a nucleic acid, a polypeptide, a drug, or an imaging agent. A plurality of the protocells can be included in a protocell composition. In some embodiments, the plurality of protocells is monodisperse, for example can have a polydispersity index of about 0.1 or less. In some embodiments, the protocells in the protocell composition have an average diameter of about 10 nm to about 250 nm, such as about 30 nm to about 100 nm. The protocell composition can be included in a pharmaceutical composition, which further comprises a pharmaceutically acceptable excipient. The pharmaceutical composition can be used to treat cancer in a subject when the protocells are loaded with an anticancer agent. The pharmaceutical composition can also be used to treat a viral infection in a subject when the protocells are loaded with an antiviral agent. The protocell can be used to deliver a cargo to a cell, such as a cancer cell or virally-infected cell. The protocell comprising the CD47 molecule or fragment thereof can also be administered to a subject to enhance in vivo circulation of the protocell. The CD47 molecule or fragment thereof can also be conjugated to the lipid bilayer of the protocell to decrease phagocytosis of the protocell by macrophages.

The protocells comprising a CD47 molecule or active fragment thereof conjugated to the lipid bilayer of the protocell, wherein the protocell is loaded with a cargo, can be used to deliver a cargo to a cell. Thus, there is provided herein a method of delivering a cargo to a cell comprising contacting a protocell to the cell, wherein the protocell is loaded with a cargo and a CD47 molecule or active fragment thereof is conjugated to the lipid bilayer of the protocell. In some embodiments, the CD47 fragment is a CD47 extracellular domain. In some embodiments, the protocell comprises an effective number of copies of the CD47 molecule or the active fragment thereof to avoid macrophage phagocytosis. For example, the protocell can comprise about 21 or more copies of the CD47 molecule or active fragment thereof. In some embodiments, the protocell further comprises a targeting species and/or a fusogenic peptide. The protocell can have a diameter between about 10 nm and about 250 nm, such as between about 30 nm and about 100 nm. In some embodiments, the protocell comprises a cargo, for example a diagnostic agent or a therapeutic agent. The cargo can be a nucleic acid, a polypeptide, a drug, or an imaging agent. A plurality of the protocells can be included in a protocell composition. In some embodiments, the plurality of protocells is monodisperse, for example can have a polydispersity index of about 0.1 or less. In some embodiments, the protocells in the protocell composition have an average diameter of about 10 nm to about 250 nm, such as about 30 nm to about 100 nm. The protocell composition can be included in a pharmaceutical composition, which further comprises a pharmaceutically acceptable excipient. The pharmaceutical composition can be used to treat cancer in a subject when the protocells are loaded with an anticancer agent. The pharmaceutical composition can also be used to treat a viral infection in a subject when the protocells are loaded with an antiviral agent. The protocell can be used to deliver a cargo to a cell, such as a cancer cell or virally-infected cell. The protocell comprising the CD47 molecule or fragment thereof can also be administered to a subject to enhance in vivo circulation of the protocell. The CD47 molecule or fragment thereof can also be conjugated to the lipid bilayer of the protocell to decrease phagocytosis of the protocell by macrophages.

Protocells (i.e., lipid coated nanoparticles) include a nanoparticulate core, such as a silica or metal oxide core, surrounded by a lipid layer, such as a lipid bilayer. Generally, the protocells are small nanoparticles that can be used to deliver a cargo to a targeted cell. Surrounding the core with a lipid layer increases cargo capacity of the core, thereby allowing the protocells to be particularly useful for the delivery of diagnostic or therapeutic agents. Good biodistribution and circulation of the protocells after in vivo administration is desirable to maximize the utility of the protocells as drug delivery vehicles. Conjugation of CD47 or an active fragment thereof increases circulation of the protocells after in vivo administration by evading uptake by macrophages.

The innate immune system of vertebrate animals uses several strategies of immune recognition that distinguish ‘self’ from ‘non-self’ to identify foreign invaders for destruction. One strategy is the ‘missing-self’ strategy of immune recognition. Specialized markers of self are expressed constitutively on normal healthy cells of the host and, by engaging inhibitory receptors, prevent phagocytosis of these cells by macrophages. Conversely, the absence of these markers on microbial cells and other foreign invaders render these targets susceptible to phagocytosis. Thus, unmarked nanoparticles administered to a subject will be identified as ‘non-self,’ resulting in phagocytosis and clearance. Previously, in vivo clearance of nanoparticles had been mitigated by conjugating the nanoparticle to a polyethylene glycol (PEG) moiety. While PEG helps to avoid non-specific protein binding and opsonization, it is not a marker of self and has been recently shown to induce an anti-PEG immune response. Additionally, the presence of PEG could sterically inhibit a targeting moiety on the protocell from binding to a target cell.

Red blood cell (RBC) membranes incorporate several markers of self, including CD47. CD47 is constitutively expressed on many cell types, including RBCs, and is a ligand for SIRP, an immunoreceptor tyrosine inhibitory motif receptor expressed on macrophages. Ligation of SIRP by CD47 prevents phagocytosis of normal cells. It has been found that conjugation of CD47 to the protocell lipid bilayer limits macrophage phagocytosis of the protocells.

The present invention provides improvements to protocell technology, to the protocells themselves, to pharmaceutical compositions which comprise such protocells and methods of using protocells and pharmaceutical compositions according to the invention for therapy and diagnostics, including monitoring therapy.

Additional embodiments of the invention relate to providing CD47 molecules on the surface of the protocells in order to enhance biodistribution and to minimize interaction with immune cells of the patient or subject to whom compositions according to the present invention are administered, and to their use in pharmaceutical compositions and methods according to other embodiments the present invention.

The following terms shall be used throughout the specification to describe the present invention. Where a term is not specifically defined herein, that term shall be understood to be used in a manner consistent with its use by those of ordinary skill in the art.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either both of those included limits are also included in the invention. In instances where a substituent is a possibility in one or more Markush groups, it is understood that only those substituents which form stable bonds are to be used.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described.

It must be noted that as used herein and in the appended claims, the singular forms “a,” “and” and “the” include plural references unless the context clearly dictates otherwise.

Reference to “about” a value or parameter herein includes (and describes) variations that are directed to that value or parameter per se. For example, description referring to “about X” includes description of “X”.

Furthermore, the following terms shall have the definitions set out below.

The term “patient” or “subject” is used throughout the specification within context to describe an animal, generally a mammal, especially including a domesticated animal and preferably a human, to whom treatment, including prophylactic treatment (prophylaxis), with the compounds or compositions according to the present invention is provided. For treatment of those infections, conditions or disease states which are specific for a specific animal such as a human patient, the term patient refers to that specific animal. In most instances, the patient or subject of the present invention is a human patient of either or both genders.

The term “effective” is used herein, unless otherwise indicated, to describe an amount of a compound or component which, when used within the context of its use, produces or effects an intended result, whether that result relates to the prophylaxis and/or therapy of an infection and/or disease state or as otherwise described herein. The term effective subsumes all other effective amount or effective concentration terms (including the term “therapeutically effective”) which are otherwise described or used in the present application.

The term “compound” is used herein to describe any specific compound or bioactive agent disclosed herein, including any and all stereoisomers (including diasteromers), individual optical isomers (enantiomers) or racemic mixtures, pharmaceutically acceptable salts and prodrug forms. The term compound herein refers to stable compounds. Within its use in context, the term compound may refer to a single compound or a mixture of compounds as otherwise described herein.

The term “bioactive agent” refers to any biologically active compound or drug which may be formulated for use in an embodiment of the present invention. Exemplary bioactive agents include the compounds according to the present invention which are used to treat cancer or a disease state or condition which occurs secondary to cancer and may include antiviral agents, especially anti-HIV, anti-HBV and/or anti-HCV agents (especially where hepatocellular cancer is to be treated) as well as other compounds or agents which are otherwise described herein.

The terms “treat”, “treating”, and “treatment”, are used synonymously to refer to any action providing a benefit to a patient at risk for or afflicted with a disease, including improvement in the condition through lessening, inhibition, suppression or elimination of at least one symptom, delay in progression of the disease, prevention, delay in or inhibition of the likelihood of the onset of the disease, etc. In the case of viral infections, these terms also apply to viral infections and preferably include, in certain particularly favorable embodiments the eradication or elimination (as provided by limits of diagnostics) of the virus which is the causative agent of the infection.

Treatment, as used herein, encompasses both prophylactic and therapeutic treatment, principally of cancer, but also of other disease states, including viral infections, especially including HBV and/or HCV. Compounds according to the present invention can, for example, be administered prophylactically to a mammal in advance of the occurrence of disease to reduce the likelihood of that disease. Prophylactic administration is effective to reduce or decrease the likelihood of the subsequent occurrence of disease in the mammal, or decrease the severity of disease (inhibition) that subsequently occurs, especially including metastasis of cancer. Alternatively, compounds according to the present invention can, for example, be administered therapeutically to a mammal that is already afflicted by disease. In one embodiment of therapeutic administration, administration of the present compounds is effective to eliminate the disease and produce a remission or substantially eliminate the likelihood of metastasis of a cancer. Administration of the compounds according to the present invention is effective to decrease the severity of the disease or lengthen the lifespan of the mammal so afflicted, as in the case of cancer, or inhibit or even eliminate the causative agent of the disease, as in the case of hepatitis B virus (HBV) and/or hepatitis C virus infections (HCV) infections.

The term “pharmaceutically acceptable” as used herein means that the compound or composition is suitable for administration to a subject, including a human patient, to achieve the treatments described herein, without unduly deleterious side effects in light of the severity of the disease and necessity of the treatment.

The term “inhibit” as used herein refers to the partial or complete elimination of a potential effect, while inhibitors are compounds/compositions that have the ability to inhibit.

The term “prevention” when used in context shall mean “reducing the likelihood” or preventing a disease, condition or disease state from occurring as a consequence of administration or concurrent administration of one or more compounds or compositions according to the present invention, alone or in combination with another agent. It is noted that prophylaxis will rarely be 100% effective; consequently the terms prevention and reducing the likelihood are used to denote the fact that within a given population of patients or subjects, administration with compounds according to the present invention will reduce the likelihood or inhibit a particular condition or disease state (in particular, the worsening of a disease state such as the growth or metastasis of cancer) or other accepted indicators of disease progression from occurring.

The term “protocell” is used to describe a porous nanoparticle which is made of a material comprising silica, polystyrene, alumina, titania, zirconia, or generally metal oxides, organometallates, organosilicates or mixtures thereof.

Porous nanoparticulates used in protocells of the invention include mesoporous silica nanoparticles and core-shell nanoparticles.

The porous nanoparticulates can also be biodegradable polymer nanoparticulates comprising one or more compositions selected from the group consisting of aliphatic polyesters, poly (lactic acid) (PLA), poly (glycolic acid) (PGA), co-polymers of lactic acid and glycolic acid (PLGA), polycarprolactone (PCL), polyanhydrides, poly(ortho)esters, polyurethanes, poly(butyric acid), poly(valeric acid), poly(lactide-co-caprolactone), alginate and other polysaccharides, collagen, and chemical derivatives thereof, albumin a hydrophilic protein, zein, a prolamine, a hydrophobic protein, and copolymers and mixtures thereof.

A porous spherical silica nanoparticle is used for the preferred protocells and is surrounded by a supported lipid or polymer bilayer or multilayer. Various embodiments according to the present invention provide nanostructures and methods for constructing and using the nanostructures and providing protocells according to the present invention. Many of the protocells in their most elemental form are known in the art. Porous silica particles of varying sizes ranging in size (diameter) from less than 5 nm to 200 nm or 500 nm or more are readily available in the art or can be readily prepared using methods known in the art (see the examples section) or alternatively, can be purchased from SkySpring Nanomaterials, Inc., Houston, Tex., USA or from Discovery Scientific, Inc., Vancouver, British Columbia. Multimodal silica nanoparticles may be readily prepared using the procedure of Carroll, et al., Langmuir, 25, 13540-13544 (2009). Protocells (i.e., lipid coated nanoparticles) can be readily obtained using methodologies known in the art. The examples section of the present application provides certain methodology for obtaining protocells which are useful in the present invention. Protocells according to the present invention may be readily prepared, including protocells comprising lipids which are fused to the surface of the silica nanoparticle. See, for example, Liu, et al., Chem. Comm., 5100-5102 (2009), Liu, et al., J. Amer. Chem. Soc., 131, 1354-1355 (2009), Liu, et al., J. Amer. Chem. Soc., 131, 7567-7569 (2009) Lu, et al., Nature, 398, 223-226 (1999), Preferred protocells for use in the present invention are prepared according to the procedures which are presented in Ashley, et al., Nature Materials, 2011, May; 10(5):389-97, Lu, et al., Nature, 398, 223-226 (1999), Caroll, et al., Langmuir, 25, 13540-13544 (2009), and as otherwise presented in the experimental section which follows.

The terms “nanoparticulate” and “porous nanoparticulate” are used interchangeably herein and such particles may exist in a crystalline phase, an amorphous phase, a semi-crystalline phase, a semi amorphous phase, or a mixture thereof.

A nanoparticle may have a variety of shapes and cross-sectional geometries that may depend, in part, upon the process used to produce the particles. In one embodiment, a nanoparticle may have a shape that is a sphere, a rod, a tube, a flake, a fiber, a plate, a wire, a cube, or a whisker. A nanoparticle may include particles having two or more of the aforementioned shapes. In one embodiment, a cross-sectional geometry of the particle may be one or more of circular, ellipsoidal, triangular, rectangular, or polygonal. In one embodiment, a nanoparticle may consist essentially of non-spherical particles. For example, such particles may have the form of ellipsoids, which may have all three principal axes of differing lengths, or may be oblate or prelate ellipsoids of revolution. Non-spherical nanoparticles alternatively may be laminar in form, wherein laminar refers to particles in which the maximum dimension along one axis is substantially less than the maximum dimension along each of the other two axes. Non-spherical nanoparticles may also have the shape of frusta of pyramids or cones, or of elongated rods. In one embodiment, the nanoparticles may be irregular in shape. In one embodiment, a plurality of nanoparticles may consist essentially of spherical nanoparticles.

The phrase “effective average particle size” as used herein to describe a multiparticulate (e.g., a porous nanoparticulate) means that at least 50% of the particles therein are of a specified size. Accordingly, “effective average particle size of less than about 2,000 nm in diameter” means that at least 50% of the particles therein are less than about 2000 nm in diameter. In certain embodiments, nanoparticulates have an effective average particle size of less than about 2,000 nm (i.e., 2 microns), less than about 1,900 nm, less than about 1,800 nm, less than about 1,700 nm, less than about 1,600 nm, less than about 1,500 nm, less than about 1,400 nm, less than about 1,300 nm, less than about 1,200 nm, less than about 1,100 nm, less than about 1,000 nm, less than about 900 nm, less than about 800 nm, less than about 700 nm, less than about 600 nm, less than about 500 nm, less than about 400 nm, less than about 300 nm, less than about 250 nm, less than about 200 nm, less than about 150 nm, less than about 100 nm, less than about 75 nm, or less than about 50 nm, as measured by light-scattering methods, microscopy, or other appropriate methods. “D₅₀” refers to the particle size below which 50% of the particles in a multiparticulate fall. Similarly, “D₉₀” is the particle size below which 90% of the particles in a multiparticulate fall. Preferred nanoparticles have an effective average particle size ranging from less than 30 nm to about 300 nm, preferably about 30 nm to about 100 nm.

The term “CD47 molecule” as used herein refers to CD47 or an active fragment thereof which can be used to inhibit immune interaction with protocells as otherwise described herein by incorporating CD47 molecules into the lipid bilayer of the protocell either by conjugation with a lipid in the fused bilayer of the protocell or by fusing/mixing a cellular plasma membrane which contains surface CD47 molecules with the lipid bilayer which is fused on the protocell. CD47 can be of any species variant, including, but not limited to, rat, mouse, or human.

CD47 (Cluster of Differentiation 47), also known as integrin associated protein (TAP) is a transmembrane protein that in humans is encoded by the CD47 gene. CD47 belongs to the immunoglobulin superfamily and partners with membrane integrins and also binds the ligands thrombospondin-1 (TSP-1) and signal-regulatory protein alpha (SIRPα). CD47 is involved in a range of cellular processes, including apoptosis, proliferation, adhesion and migration and plays a key role in immune and angiogenic responses. CD47 is ubiquitously expressed in human cells and has found to be overexpressed in many different tumor cells. The present application incorporates CD47 molecules and active fragments thereof into lipid bilayers of protocell either through conjugation with lipids in the bilayers or by incorporating/fusing cell plasma membrane which contain CD47 molecules into the lipid bilayer of the protocell. The resulting protocells exhibit much lower interaction with immune cells after administration, thus promoting bioavailability and distribution which exceeds protocells which do not incorporate CD47.

### CD47 Conjugated to a Lipid Bilayer

The protocells described herein include a core (i.e., frequently referred to herein as a “nanoparticulate” or “nanoparticle” core) surrounded by a lipid bilayer. Generally, the lipid bilayer is a supported lipid bilayer. CD47, or an active fragment thereof, is conjugated to the lipid bilayer. In some embodiments, the CD47 fragment is a CD47 extracellular domain or a fragment of a CD47 extracellular domain. Optionally, the protocells (i.e., lipid coated nanoparticles) can further comprise a targeting species, an endosomolytic peptide, or a cargo, as further described herein.

Use of CD47 conjugated to the lipid bilayer is generally preferred over the use of erythrocyte membranes comprising CD47 because conjugation of isolated CD47 limits the number of unessential components on the surface of the protocell. This helps limit non-specific interactions and adverse reactions when the protocells are administered in vivo. Thus, in some embodiments, the lipid bilayer is a synthetic lipid bilayer. As used herein, the term “synthetic lipid bilayer” refers to a bilayer that is not derived from a cellular plasma membrane. The CD47 conjugated to the lipid bilayer can be recombinant CD47 to provide increased purity.

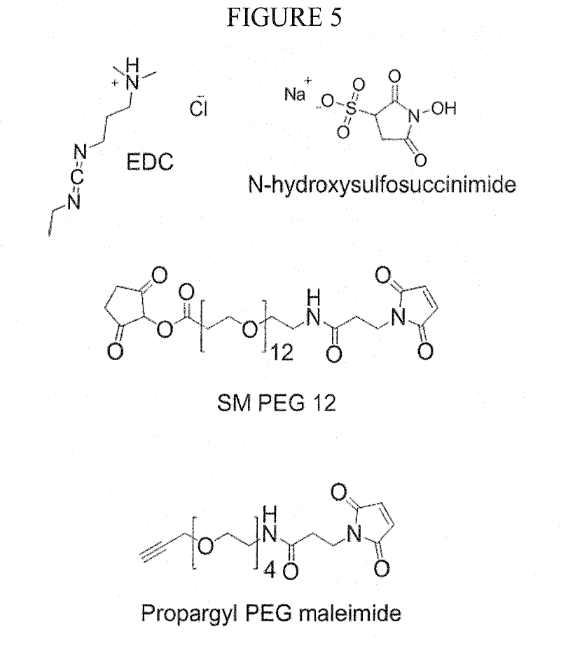

CD47 or an active fragment can be conjugated to the lipid bilayer using a linker, such as a heterobifunctional linker or a click chemistry linker. Exemplary heterobifunctional crosslinkers include an amine-to-sulfhydryl crosslinker or an amine-to-carboxylic acid crosslinker. The heterobifunctional crosslinker can include a spacer (such as a PEG spacer), which increases the distance between the CD47 molecule or active fragment thereof and the lipid bilayer. In some embodiments, the PEG spacer is between 2 and 24 units in length (such as between 2 and 12 units in length, between 4 and 8 units in length, or 6 units in length). Extending the number of units of PEG in the SM-PEGₙ alters the separation distance between the lipid bilayer and the CD47 molecule or active fragment. Thus, the linker can be between about 15 Å and about 100 Å in length (such as between about 30 Å and about 80 Å in length, or between about 40 Å and about 60 Å in length). FIG. 5 presents examples of crosslinkers that can be used to conjugate CD47 or an active fragment thereof to the lipid bilayer.

One example of an amine-to-sulfhydryl crosslinker comprises a maleimide reactive group and an N-hydroxysuccinimide ester (NHS) reactive groups separated by a PEG spacer (often referred to as SM-PEGₙ, wherein “n” denotes the number of PEG spacer units). Various SM-PEGₙ compounds are available from ThermoFisher Scientific, for example SM-PEG₂, SM-PEG₄, SM-PEG₆, SM-PEG₈, SM-PEG₁₂, and SM-PEG₂₄. The amine-to-sulfhydryl crosslinker reacts with a sulfhydryl moiety (such as a cysteine residue) present on CD47 or active fragment thereof, and an amine moiety on an amine-functionalized lipid, such as DSPE-PEG-Amine or any of the other amine-functionalized lipids disclosed herein.

The amine-to-carboxylic acid crosslinker reacts with a carboxylic acid moiety present in CD47 or active fragment thereof, and an amine moiety on an amine-functionalized lipid. One example of an amine-to-carboxylic acid crosslinker is ethyl(dimethylaminopropyl) carbodiimide (EDC), which can be further activated for better crosslinking efficiency using N-hydroxylsuflosuccinimide (sulfo-NHS).

Click chemistry crosslinkers can also be used to conjugate CD47 or an active fragment thereof to the lipid bilayer. One example of a click chemistry crosslinker is propargyl-PEG-maleimide. The click chemistry crosslinker can react with a sulfhydryl moiety (such as a cysteine reside) present on CD47 or active fragment thereof and an azide functionalized lipid, such as DSPE-PEG-Azide, present in the lipid bilayer.

In another example, CD47 or an active fragment thereof can be conjugated to the lipid bilayer by incorporating a chelating lipid (such as NTA/Ni chelating lipid, such as DOGS-NTA/Ni). The chelating lipid can comprise a nitrilotriacetic acid moiety (NTA) or an iminodiacetic acid (IDA) moiety, which chelates a divalent cation. The CD47 or an active fragment thereof can include a polyhistidine tag (commonly referred to as a “His-tag”) at the C-terminus or N-terminus of the protein. The His-tag can the bind to the chelating lipid to conjugate the CD47 or active fragment thereof to the lipid bilayer.

The protocells comprising CD47 conjugated to the lipid bilayer can be monodisperse. For example, the protocells can have a polydispersity index of about 0.2 or less, about 0.15 or less, about 0.1 or less, about 0.09 or less, about 0.08 or less, or about 0.07 or less.

In some embodiments, for more effective macrophage evasion, the protocells are conjugated to about 21 or more copies of CD47 or an active fragment thereof (such about 30 or more copies, about 40 or more copies, about 80 or more copies, about 120 or more copies, about 150 or more copies, about 170 or more copies, about 200 or more copies, about 250 or more copies, about 300 or more copies, about 400 or more copies, or about 500 or more copies) per protocell.

A plurality of protocells comprising a CD47 molecule conjugated to the lipid bilayer can be included in a pharmaceutical composition, which further comprises a pharmaceutically acceptable excipient. The protocells can also be loaded with an anticancer agent and used to treat cancer in a patient. In another embodiment, the protocells are loaded with an antiviral agent and used to treat a viral infection in a patient.

The protocells comprising a CD47 molecule conjugated to the lipid bilayer can also be used to deliver a cargo to cell, such as a cancer cell or a virally infected cell, by contacting the protocell with the cell.

CD47 conjugated to the lipid bilayer of the protocell enhances in vivo circulation of the protocell after administration to subject. Thus, a protocell comprising a core surrounded by a lipid bilayer, wherein a CD47 molecule is conjugated to the lipid bilayer can be administered to a subject for enhanced in vivo circulation of the protocell.

Protocells with CD47 conjugated to the lipid bilayer result in decreased macrophage uptake. Thus, there is provided a method of decreasing uptake protocells by macrophages comprising conjugating a CD47 molecule to the lipid bilayer of the protocell.

In certain embodiments, the porous nanoparticulates are comprised of one or more compositions selected from the group consisting of silica, a biodegradable polymer, a solgel, a metal and a metal oxide.

In an embodiment of the present invention, the nanostructures include a core-shell structure which comprises a porous particle core surrounded by a shell of lipid preferably a bilayer, but possibly a monolayer or multilayer (see Liu, et al., JACS, 2009, Id). The porous particle core can include, for example, a porous nanoparticle made of an inorganic and/or organic material as set forth above surrounded by a lipid bilayer. In the present invention, these lipid bilayer surrounded nanostructures are referred to as “protocells” or “functional protocells,” since they have a supported lipid bilayer membrane structure. In embodiments according to the present invention, the porous particle core of the protocells can be loaded with various desired species (“cargo”), including small molecules (e.g. anticancer agents as otherwise described herein), large molecules (e.g. including macromolecules such as RNA, including small interfering RNA or siRNA or small hairpin RNA or shRNA or a polypeptide which may include a polypeptide toxin such as a ricin toxin A-chain or other toxic polypeptide such as diphtheria toxin A-chain DTx, among others) or a reporter polypeptide (e.g. fluorescent green protein, among others) or semiconductor quantum dots, or metallic nanoparticles, or metal oxide nanoparticles or combinations thereof. In certain preferred aspects of the invention, the protocells are loaded with super-coiled plasmid DNA, which can be used to deliver a therapeutic and/or diagnostic peptide(s) or a small hairpin RNA/shRNA or small interfering RNA/siRNA which can be used to inhibit expression of proteins (such as, for example growth factor receptors or other receptors which are responsible for or assist in the growth of a cell especially a cancer cell, including epithelial growth factor/EGFR, vascular endothelial growth factor receptor/VEGFR-2 or platelet derived growth factor receptor/PDGFR-α, among numerous others, and induce growth arrest and apoptosis of cancer cells).

In certain embodiments, the cargo components can include, but are not limited to, chemical small molecules (especially anticancer agents and antiviral agents, including anti-HIV, anti-HBV and/or anti-HCV agents, nucleic acids (DNA and RNA, including siRNA and shRNA and plasmids which, after delivery to a cell, express one or more polypeptides or RNA molecules), such as for a particular purpose, such as a therapeutic application or a diagnostic application as otherwise disclosed herein.

In embodiments, the lipid bilayer of the protocells can provide biocompatibility and can be modified to possess targeting species including, for example, targeting peptides including antibodies, aptamers, and PEG (polyethylene glycol) to allow, for example, further stability of the protocells and/or a targeted delivery into a bioactive cell.

In preferred embodiments, the lipid bilayer is modified to contain CD47 molecules either by conjugation to lipids within the bilayer or by incorporation of a cellular plasma membrane into the lipid bilayer along with traditional lipids as otherwise disclosed herein.

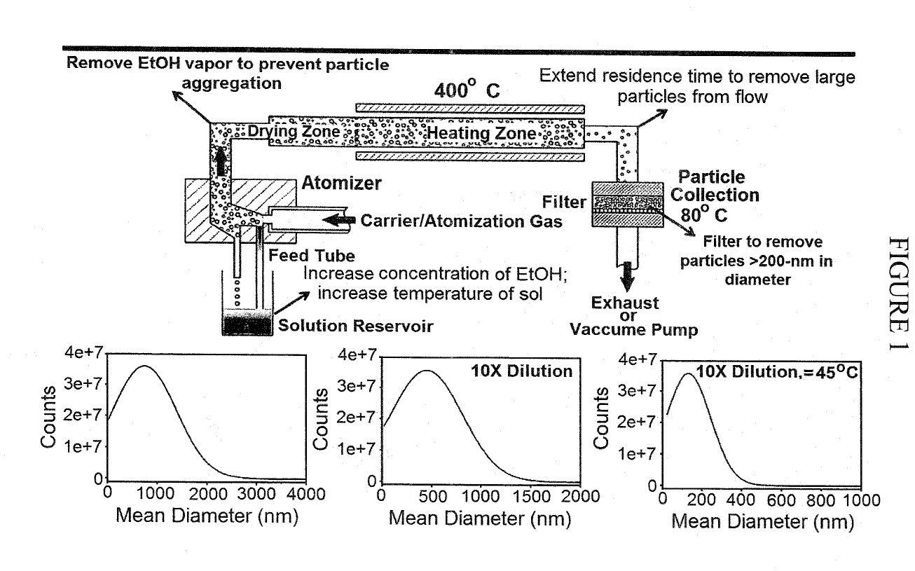

The protocells particle size distribution, according to the present invention, depending on the application, may be monodisperse or polydisperse. The silica cores can be rather monodisperse (i.e., a uniform sized population varying no more than about 5% in diameter e.g., ±10-nm for a 200 nm diameter protocell especially if they are prepared using solution techniques) or rather polydisperse (i.e., a polydisperse population can vary widely from a mean or medium diameter, e.g., up to ±200-nm or more if prepared by aerosol. See FIG. 1. Polydisperse populations can be sized into monodisperse populations. All of these are suitable for protocell formation. In the present invention, preferred protocells are preferably no more than about 500 nm in diameter, preferably no more than about 200 nm in diameter in order to afford delivery to a patient or subject and produce an intended therapeutic effect.

In certain embodiments, protocells according to the present invention generally range in size from greater than about 8-10 nm to about 5 μm in diameter, preferably about 20-nm-3 μm in diameter, about 10 nm to about 500 nm, more preferably about 20-200-nm (including about 150 nm, which may be a mean or median diameter). In certain embodiments, the protocells have a diameter between about 30 nm to about 100 nm. As discussed above, the protocell population may be considered monodisperse or polydisperse based upon the mean or median diameter of the population of protocells. Size is very important to therapeutic and diagnostic aspects of the present invention as particles smaller than about 8-nm diameter are excreted through kidneys, and those particles larger than about 200 nm are trapped by the liver and spleen. Thus, an embodiment of the present invention focuses in smaller sized protocells for drug delivery and diagnostics in the patient or subject.

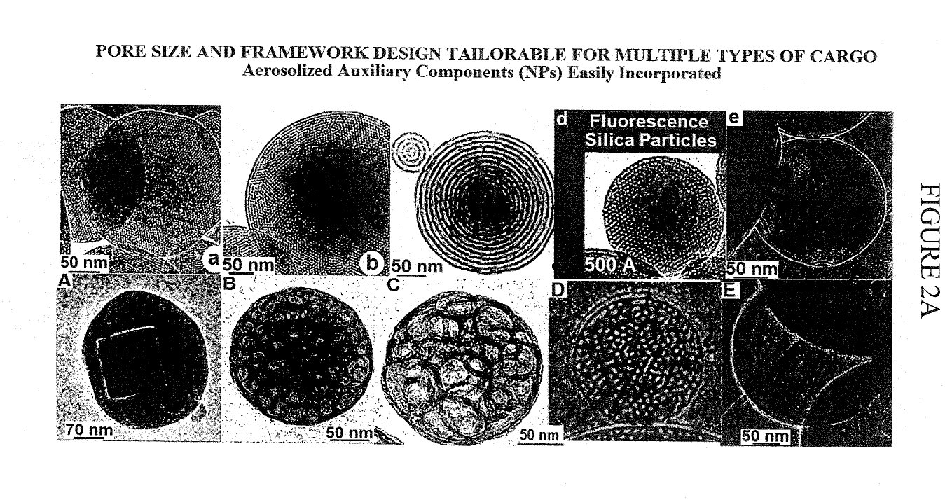

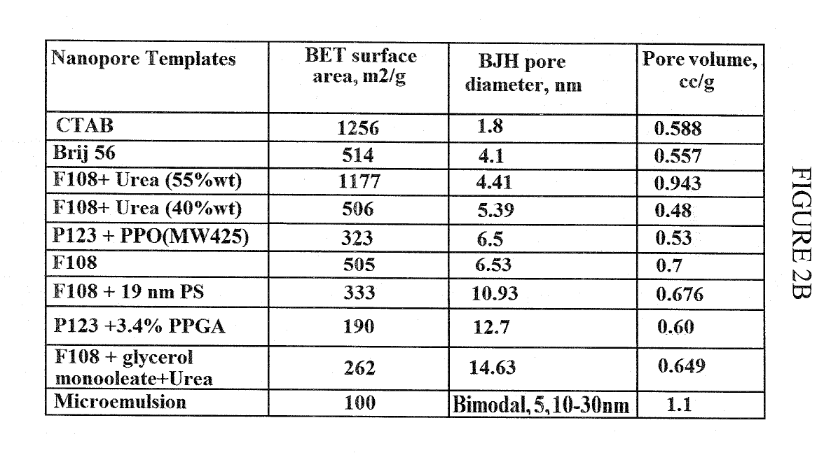

In certain embodiments, protocells according the present invention are characterized by containing mesopores, preferably pores which are found in the nanostructure material. These pores (at least one, but often a large plurality) may be found intersecting the surface of the nanoparticle (by having one or both ends of the pore appearing on the surface of the nanoparticle) or internal to the nanostructure with at least one or more mesopore interconnecting with the surface mesopores of the nanoparticle. Interconnecting pores of smaller size are often found internal to the surface mesopores. The overall range of pore size of the mesopores can be 0.03-50-nm in diameter. Preferred pore sizes of mesopores range from about 2-30 nm; they can be monosized or bimodal or graded—they can be ordered or disordered (essentially randomly disposed or worm-like). See FIGS. 2A and 2B.

Mesopores (IUPAC definition 2-50-nm in diameter) are ‘molded’ by templating agents including surfactants, block copolymers, molecules, macromolecules, emulsions, latex beads, or nanoparticles. In addition, processes could also lead to micropores (IUPAC definition less than 2-nm in diameter) all the way down to about 0.03-nm e.g. if a templating moiety in the aerosol process is not used. They could also be enlarged to macropores, i.e., 50-nm in diameter.

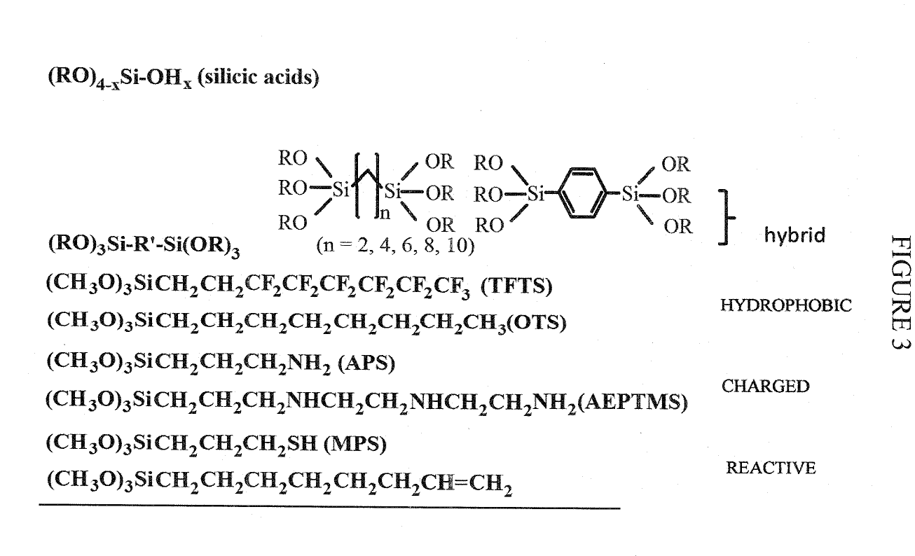

Pore surface chemistry of the nanoparticle material can be very diverse—all organosilanes yielding cationic, anionic, hydrophilic, hydrophobic, reactive groups—pore surface chemistry, especially charge and hydrohobicity, affect loading capacity. See FIG. 3. Attractive electrostatic interactions or hydrophobic interactions control/enhance loading capacity and control release rates. Higher surface areas can lead to higher loadings of drugs/cargos through these attractive interactions. See below.

In certain embodiments, the surface area of nanoparticles, as measured by the N2 BET method, ranges from about 100 m²/g to about 1200 m²/g. In general, the larger the pore size, the smaller the surface area. See FIG. 2B. The surface area theoretically could be reduced to essentially zero, if one does not remove the templating agent or if the pores are sub-0.5-nm and therefore not measurable by N₂ sorption at 77K due to kinetic effects. However, in this case, they could be measured by CO₂ or water sorption, but would probably be considered non-porous. This would apply if biomolecules are encapsulated directly in the silica cores prepared without templates, in which case particles (internal cargo) would be released by dissolution of the silica matrix after delivery to the cell.

Typically the protocells according to the present invention are loaded with cargo to a capacity up to over 100 weight %: defined as (cargo weight/weight of protocell)×100. The optimal loading of cargo is often about 0.01 to 30% but this depends on the drug or drug combination which is incorporated as cargo into the protocell. This is generally expressed in μM per 10¹⁰ particles where we have values ranging from 2000-100 μM per 10¹⁰ particles. In some protocells according to the present invention exhibit release of cargo at pH about 5.5, which is that of the endosome, but are stable at physiological pH of 7 or higher (7.4).

The surface area of the internal space for loading is the pore volume whose optimal value ranges from about 1.1 to 0.5 cubic centimeters per gram (cc/g). Note that in the protocells according to one embodiment of the present invention, the surface area is mainly internal as opposed to the external geometric surface area of the nanoparticle.

The lipid bilayer supported on the porous particle according to one embodiment of the present invention has a lower melting transition temperature, i.e. is more fluid than a lipid bilayer supported on a non-porous support or the lipid bilayer in a liposome. This is sometimes important in achieving high affinity binding of targeting ligands at low peptide densities, as it is the bilayer fluidity that allows lateral diffusion and recruitment of peptides by target cell surface receptors. One embodiment provides for peptides to cluster, which facilitates binding to a complementary target.

In the present invention, the lipid bilayer may vary significantly in composition. Ordinarily, any lipid or polymer which is may be used in liposomes may also be used in protocells. Preferred lipids are as otherwise described herein. Particularly preferred lipid bilayers for use in protocells according to the present invention comprise a mixtures of lipids (as otherwise described herein) at a weight ratio of 5% DOPE, 5% PEG, 30% cholesterol, 60% DOPC or DPPC (by weight).

The charge of the mesoporous silica NP core as measured by the Zeta potential may be varied monotonically from −50 to +50 mV by modification with the amine silane, 2-(aminoethyl) propyltrimethoxy-silane (AEPTMS) or other organosilanes. This charge modification, in turn, varies the loading of the drug within the cargo of the protocell. Generally, after fusion of the supported lipid bilayer, the zeta-potential is reduced to between about −10 mV and +5 mV, which is important for maximizing circulation time in the blood and avoiding non-specific interactions.

Depending on how the surfactant template is removed, e.g. calcination at high temperature (500° C.) versus extraction in acidic ethanol, and on the amount of AEPTMS incorporated in the silica framework, the silica dissolution rates can be varied widely. This in turn controls the release rate of the internal cargo. This occurs because molecules that are strongly attracted to the internal surface area of the pores diffuse slowly out of the particle cores, so dissolution of the particle cores controls in part the release rate.

Further characteristics of protocells according to an embodiment of the present invention are that they are stable at pH 7, i.e. they don't leak their cargo, but at pH 5.5, which is that of the endosome lipid or polymer coating becomes destabilized initiating cargo release. This pH-triggered release is important for maintaining stability of the protocell up until the point that it is internalized in the cell by endocytosis, whereupon several pH triggered events cause release into the endosome and consequently, the cytosol of the cell. The protocell core particle and surface can also be modified to provide non-specific release of cargo over a specified, prolonged period of time, as well as be reformulated to release cargo upon other biophysical changes, such as the increased presence of reactive oxygen species and other factors in locally inflamed areas. Quantitative experimental evidence has shown that targeted protocells illicit only a weak immune response, because they do not support T-Cell help required for higher affinity IgG, a favorable result.

Protocells according to the present invention in some embodiments exhibit one or more a number of characteristics (depending upon the embodiment) which distinguish them from prior art protocells:
1) In contrast to the prior art, an embodiment of the present invention specifies nanoparticles whose average size (diameter) is less than about 200-nm—this size is engineered to enable efficient cellular uptake by receptor mediated endocytosis and to minimize binding and uptake by non-target cells and organs;
2) An embodiment of the present invention can specify both monodisperse and/or polydisperse sizes to enable control of biodistribution.
3) An embodiment of the present invention is directed to targeted nanoparticles that induce receptor mediated endocytosis.
4) An embodiment of the present invention induces dispersion of cargo into cytoplasm through the inclusion of fusogenic or endosomolytic peptides.
5) An embodiment of the present invention provides particles with pH triggered release of cargo.
6) An embodiment of the present invention exhibits controlled time dependent release of cargo (via extent of thermally induced crosslinking of silica nanoparticle matrix).
7) An embodiment of the present invention can exhibit time dependent pH triggered release.
8) An embodiment of the present invention can contain and provide cellular delivery of complex multiple cargoes.
9) An embodiment of the present invention shows the killing of target cancer cells.
10) An embodiment of the present invention shows diagnosis of target cancer cells.
11) An embodiment of the present invention shows selective entry of target cells.
12) An embodiment of the present invention shows selective exclusion from off-target cells (selectivity).
13) An embodiment of the present invention shows enhanced fluidity of the supported lipid bilayer.
14) An embodiment of the present invention exhibits sub-nanomolar and controlled binding affinity to target cells.
15) An embodiment of the present invention exhibits sub-nanomolar binding affinity with targeting ligand densities below concentrations found in the prior art.
16) An embodiment of the present invention can further distinguish the prior art with finer levels of detail unavailable in the prior art.

The term “lipid” is used to describe the components which are used to form lipid bilayers on the surface of the nanoparticles which are used in the present invention. Various embodiments provide nanostructures which are constructed from nanoparticles which support a lipid bilayer(s). In embodiments according to the present invention, the nanostructures preferably include, for example, a core-shell structure including a porous particle core surrounded by a shell of lipid bilayer(s). The nanostructure, preferably a porous silica nanostructure as described above, supports the lipid bilayer membrane structure. In embodiments according to the invention, the lipid bilayer of the protocells can provide biocompatibility and can be modified to possess targeting species including, for example, targeting peptides, fusogenic peptides, antibodies, aptamers, and PEG (polyethylene glycol) to allow, for example, further stability of the protocells and/or a targeted delivery into a bioactive cell, in particular a cancer cell. PEG, when included in lipid bilayers, can vary widely in molecular weight (although PEG ranging from about 10 to about 100 units of ethylene glycol, about 15 to about 50 units, about 15 to about 20 units, about 15 to about 25 units, about 16 to about 18 units, etc, may be used and the PEG component which is generally conjugated to phospholipid through an amine group comprises about 1% to about 20%, preferably about 5% to about 15%, about 10% by weight of the lipids which are included in the lipid bilayer. One or more of the lipids used in the fused lipid bilayer of the protocells of the present invention may be conjugated to CD47 molecules and incorporated into the lipid bilayer. Alternatively, the lipid bilayer may be mixed or fused with a cellular plasma membrane (often from red blood cells) which contain CD47 molecules.

Numerous lipids which are used in liposome delivery systems may be used to form the lipid bilayer on nanoparticles to provide protocells according to the present invention. Virtually any lipid or polymer which is used to form a liposome or polymersome may be used in the lipid bilayer which surrounds the nanoparticles to form protocells according to an embodiment of the present invention. Preferred lipids for use in the present invention include, for example, 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-dioleoyl-sn-glycero-3-[phosphor-L-serine] (DOPS), 1,2-dioleoyl-3-trimethylammonium-propane (18:1 DOTAP), 1,2-dioleoyl-sn-glycero-3-phospho-(1′-rac-glycerol) (DOPG), 1,2-dioleoyl-sn-glycero-3-phosphoethanolamine (DOPE), 1,2-dipalmitoyl-sn-glycero-3-phosphoethanolamine (DPPE), 1,2-dioleoyl-sn-glycero-3-phosphoethanolamine-N-[methoxy(polyethylene glycol)-2000] (18:1 PEG-2000 PE), 1,2-dipalmitoyl-sn-glycero-3-phosphoethanolamine-N-[methoxy(polyethylene glycol)-2000] (16:0 PEG-2000 PE), 1-Oleoyl-2-[12-[(7-nitro-2-1,3-benzoxadiazol-4-yl)amino]lauroyl]-sn-Glycero-3-Phosphocholine (18:1-12:0 NBD PC), 1-palmitoyl-2-{12-[(7-nitro-2-1,3-benzoxadiazol-4-yl)amino]lauroyl}-sn-glycero-3-phosphocholine (16:0-12:0 NBD PC), cholesterol and mixtures/combinations thereof. Cholesterol, not technically a lipid, but presented as a lipid for purposes of an embodiment of the present invention given the fact that cholesterol may be an important component of the lipid bilayer of protocells according to an embodiment of the invention. Often cholesterol is incorporated into lipid bilayers of protocells in order to enhance structural integrity of the bilayer. These lipids are all readily available commercially from Avanti Polar Lipids, Inc. (Alabaster, Ala., USA). DOPE and DPPE are particularly useful for conjugating (through an appropriate crosslinker) peptides, polypeptides, including antibodies, RNA and DNA through the amine group on the lipid.

In certain embodiments, the porous nanoparticulates can also be biodegradable polymer nanoparticulates comprising one or more compositions selected from the group consisting of aliphatic polyesters, poly (lactic acid) (PLA), poly (glycolic acid) (PGA), co-polymers of lactic acid and glycolic acid (PLGA), polycarprolactone (PCL), polyanhydrides, poly(ortho)esters, polyurethanes, poly(butyric acid), poly(valeric acid), poly(lactide-co-caprolactone), alginate and other polysaccharides, collagen, and chemical derivatives thereof, albumin a hydrophilic protein, zein, a prolamine, a hydrophobic protein, and copolymers and mixtures thereof.

In still other embodiments, the porous nanoparticles each comprise a core having a core surface that is essentially free of silica, and a shell attached to the core surface, wherein the core comprises a transition metal compound selected from the group consisting of oxides, carbides, sulfides, nitrides, phosphides, borides, halides, selenides, tellurides, tantalum oxide, iron oxide or combinations thereof.

The silica nanoparticles used in the present invention can be, for example, mesoporous silica nanoparticles and core-shell nanoparticles. The nanoparticles may incorporate an absorbing molecule, e.g. an absorbing dye. Under appropriate conditions, the nanoparticles emit electromagnetic radiation resulting from chemiluminescence. Additional contrast agents may be included to facilitate contrast in MRI, CT, PET, and/or ultrasound imaging.

Mesoporous silica nanoparticles can be e.g. from around 5 nm to around 500 nm in size, including all integers and ranges there between. The size is measured as the longest axis of the particle. In various embodiments, the particles are from around 10 nm to around 500 nm, from around 10 nm to around 100 nm in size, or from around 30 nm to around 100 nm in size. The mesoporous silica nanoparticles have a porous structure. The pores can be from around 1 to around 20 nm in diameter, including all integers and ranges there between. In one embodiment, the pores are from around 1 to around 10 nm in diameter. In one embodiment, around 90% of the pores are from around 1 to around 20 nm in diameter. In another embodiment, around 95% of the pores are around 1 to around 20 nm in diameter.

The mesoporous nanoparticles can be synthesized according to methods known in the art. In one embodiment, the nanoparticles are synthesized using sol-gel methodology where a silica precursor or silica precursors and a silica precursor or silica precursors conjugated (i.e., covalently bound) to absorber molecules are hydrolyzed in the presence of templates in the form of micelles. The templates are formed using a surfactant such as, for example, hexadecyltrimethylammonium bromide (CTAB). It is expected that any surfactant which can form micelles can be used.

The core-shell nanoparticles comprise a core and shell. The core comprises silica and an absorber molecule. The absorber molecule is incorporated in to the silica network via a covalent bond or bonds between the molecule and silica network. The shell comprises silica.

In one embodiment, the core is independently synthesized using known sol-gel chemistry, e.g., by hydrolysis of a silica precursor or precursors. The silica precursors are present as a mixture of a silica precursor and a silica precursor conjugated, e.g., linked by a covalent bond, to an absorber molecule (referred to herein as a “conjugated silica precursor”). Hydrolysis can be carried out under alkaline (basic) conditions to form a silica core and/or silica shell. For example, the hydrolysis can be carried out by addition of ammonium hydroxide to the mixture comprising silica precursor(s) and conjugated silica precursor(s).

Silica precursors are compounds which under hydrolysis conditions can form silica. Examples of silica precursors include, but are not limited to, organosilanes such as, for example, tetraethoxysilane (TEOS), tetramethoxysilane (TMOS) and the like.

The silica precursor used to form the conjugated silica precursor has a functional group or groups which can react with the absorbing molecule or molecules to form a covalent bond or bonds. Examples of such silica precursors include, but is not limited to, isocyanatopropyltriethoxysilane (ICPTS), aminopropyltrimethoxysilane (APTS), mercaptopropyltrimethoxysilane (MPTS), and the like.

In one embodiment, an organosilane (conjugatable silica precursor) used for forming the core has the general formula R₄ₙSiXₙ, where X is a hydrolyzable group such as ethoxy, methoxy, or 2-methoxy-ethoxy; R can be a monovalent organic group of from 1 to 12 carbon atoms which can optionally contain, but is not limited to, a functional organic group such as mercapto, epoxy, acrylyl, methacrylyl, or amino; and n is an integer of from 0 to 4. The conjugatable silica precursor is conjugated to an absorber molecule and subsequently co-condensed for forming the core with silica precursors such as, for example, TEOS and TMOS. A silane used for forming the silica shell has n equal to 4. The use of functional mono-, bis- and tris-alkoxysilanes for coupling and modification of co-reactive functional groups or hydroxy-functional surfaces, including glass surfaces, is also known, see Kirk-Othmer, Encyclopedia of Chemical Technology, Vol. 20, 3rd Ed., J. Wiley, N.Y.; see also E. Pluedemann, Silane Coupling Agents, Plenum Press, N.Y. 1982. The organo-silane can cause gels, so it may be desirable to employ an alcohol or other known stabilizers. Processes to synthesize core-shell nanoparticles using modified Stoeber processes can be found in U.S. patent application Ser. Nos. 10/306,614 and 10/536,569, the disclosure of such processes therein are incorporated herein by reference.

“Amine-containing silanes” include, but are not limited to, a primary amine, a secondary amine or a tertiary amine functionalized with a silicon atom, and may be a monoamine or a polyamine such as diamine. Preferably, the amine-containing silane is N-(2-aminoethyl)-3-aminopropyltrimethoxysilane (AEPTMS). Non-limiting examples of amine-containing silanes also include 3-aminopropyltrimethoxysilane (APTMS) and 3-aminopropyltriethoxysilane (APTS), as well as an amino-functional trialkoxysilane. Protonated secondary amines, protonated tertiary alkyl amines, protonated amidines, protonated guanidines, protonated pyridines, protonated pyrimidines, protonated pyrazines, protonated purines, protonated imidazoles, protonated pyrroles, quaternary alkyl amines, or combinations thereof, can also be used.

In certain embodiments of a protocell of the invention, the lipid bilayer is comprised of one or more lipids selected from the group consisting of phosphatidyl-cholines (PCs) and cholesterol.

In certain embodiments, the lipid bilayer is comprised of one or more phosphatidyl-cholines (PCs) selected from the group consisting of 1,2-dimyristoyl-sn-glycero-3-phosphocholine (DMPC), 1,2-dioleoyl-3-trimethylammonium-propane (DOTAP), 1-palmitoyl-2-oleoyl-sn-glycero-3-phosphocholine (POPC), egg PC, and a lipid mixture comprising between about 50% to about 70%, or about 51% to about 69%, or about 52% to about 68%, or about 53% to about 67%, or about 54% to about 66%, or about 55% to about 65%, or about 56% to about 64%, or about 57% to about 63%, or about 58% to about 62%, or about 59% to about 61%, or about 60%, of one or more unsaturated phosphatidyl-cholines, DMPC [14:0] having a carbon length of 14 and no unsaturated bonds, 1,2-dipalmitoyl-sn-glycero-3-phosphocholine (DPPC) [16:0], 1,2-distearoyl-sn-glycero-3-phosphocholine (DSPC) [18:0], 1,2-dioleoyl-sn-glycero-3-phosphocholine (DOPC) [18:1 (Δ9-Cis)], POPC [16:0-18:1], and DOTAP [18:1].

In other embodiments:

(a) the lipid bilayer is comprised of a mixture of (1) egg PC, and (2) one or more phosphatidyl-cholines (PCs) selected from the group consisting of 1,2-dimyristoyl-sn-glycero-3-phosphocholine (DMPC), 1,2-dioleoyl-3-trimethylammonium-propane (DOTAP), 1-palmitoyl-2-oleoyl-sn-glycero-3-phosphocholine (POPC), a lipid mixture comprising between about 50% to about 70% or about 51% to about 69%, or about 52% to about 68%, or about 53% to about 67%, or about 54% to about 66%, or about 55% to about 65%, or about 56% to about 64%, or about 57% to about 63%, or about 58% to about 62%, or about 59% to about 61%, or about 60%, of one or more unsaturated phosphatidyl-choline, DMPC [14:0] having a carbon length of 14 and no unsaturated bonds, 1,2-dipalmitoyl-sn-glycero-3-phosphocholine (DPPC) [16:0], 1,2-distearoyl-sn-glycero-3-phosphocholine (DSPC) [18:0], 1,2-dioleoyl-sn-glycero-3-phosphocholine (DOPC) [18:1 (Δ9-Cis)], POPC [16:0-18:1] and DOTAP [18:1]; and wherein
(b) the molar concentration of egg PC in the mixture is between about 10% to about 50% or about 11% to about 49%, or about 12% to about 48%, or about 13% to about 47%, or about 14% to about 46%, or about 15% to about 45%, or about 16% to about 44%, or about 17% to about 43%, or about 18% to about 42%, or about 19% to about 41%, or about 20% to about 40%, or about 21% to about 39%, or about 22% to about 38%, or about 23% to about 37%, or about 24% to about 36%, or about 25% to about 35%, or about 26% to about 34%, or about 27% to about 33%, or about 28% to about 32%, or about 29% to about 31%, or about 30%.

In certain embodiments, the lipid bilayer is comprised of one or more compositions selected from the group consisting of a phospholipid, a phosphatidyl-choline, a phosphatidyl-serine, a phosphatidyl-diethanolamine, a phosphatidylinosite, a sphingolipid, and an ethoxylated sterol, or mixtures thereof. In illustrative examples of such embodiments, the phospholipid can be a lecithin; the phosphatidylinosite can be derived from soy, rape, cotton seed, egg and mixtures thereof; the sphingolipid can be ceramide, a cerebroside, a sphingosine, and a sphingomyelin, and a mixture thereof; the ethoxylated sterol can be phytosterol, PEG-(polyethyleneglykol)-5-soy bean sterol, and PEG-(polyethyleneglykol)-5 rapeseed sterol. In certain embodiments, the phytosterol comprises a mixture of at least two of the following compositions: sistosterol, camposterol and stigmasterol.

In still other illustrative embodiments, the lipid bilayer is comprised of one or more phosphatidyl groups selected from the group consisting of phosphatidyl choline, phosphatidyl-ethanolamine, phosphatidyl-serine, phosphatidyl-inositol, lyso-phosphatidyl-choline, lyso-phosphatidyl-ethanolamnine, lyso-phosphatidyl-inositol and lyso-phosphatidyl-inositol.

In still other illustrative embodiments, the lipid bilayer is comprised of phospholipid selected from a monoacyl or diacylphosphoglyceride.

In still other illustrative embodiments, the lipid bilayer is comprised of one or more phosphoinositides selected from the group consisting of phosphatidyl-inositol-3-phosphate (PI-3-P), phosphatidyl-inositol-4-phosphate (PI-4-P), phosphatidyl-inositol-5-phosphate 5-P), phosphatidyl-inositol-3,4-diphosphate (PI-3,4-P2), phosphatidyl-inositol-3,5-diphosphate (PI-3,5-P2), phosphatidyl-inositol-4,5-diphosphate (PI-4,5-P2), phosphatidyl-inositol-3,4,5-triphosphate (PI-3,4,5-P3), lysophosphatidyl-inositol-3-phosphate (LPI-3-P), lysophosphatidyl-inositol-4-phosphate (LPI-4-P), lysophosphatidyl-inositol-5-phosphate (LPI-5-P), lysophosphatidyl-inositol-3,4-diphosphate (LPI-3,4-P2), lysophosphatidyl-inositol-3,5-diphosphate (LPI-3,5-P2), lysophosphatidyl-inositol-4,5-diphosphate (LPI-4,5-P2), and lysophosphatidyl-inositol-3,4,5-triphosphate (LPI-3,4,5-P3), and phosphatidyl-inositol (PI), and lysophosphatidyl-inositol (LPI).

In still other illustrative embodiments, the lipid bilayer is comprised of one or more phospholipids selected from the group consisting of PEG-poly(ethylene glycol)-derivatized distearoylphosphatidylethanolamine (PEG-DSPE), poly(ethylene glycol)-derivatized ceramides (PEG-CER), hydrogenated soy phosphatidylcholine (HSPC), egg phosphatidylcholine (EPC), phosphatidyl ethanolamine (PE), phosphatidyl glycerol (PG), phosphatidyl insitol (PI), monosialogangolioside, spingomyelin (SPM), distearoylphosphatidylcholine (DSPC), dimyristoylphosphatidylcholine (DMPC), and dimyristoylphosphatidylglycerol (DMPG).

In one illustrative embodiment of a protocell of the invention:

(a) the one or more pharmaceutically-active agents include at least one anticancer agent;
(b) less than around 10% to around 20% of the anticancer agent is released from the porous nanoparticulates in the absence of a reactive oxygen species; and
(c) upon disruption of the lipid bilayer as a result of contact with a reactive oxygen species, the porous nanoparticulates release an amount of anticancer agent that is approximately equal to around 60% to around 80%, or around 61% to around 79%, or around 62% to around 78%, or around 63% to around 77%, or around 64% to around 77%, or around 65% to around 76%, or around 66% to around 75%, or around 67% to around 74%, or around 68% to around 73%, or around 69% to around 72%, or around 70% to around 71%, or around 70% of the amount of anticancer agent that would have been released had the lipid bilayer been lysed with 5% (w/v) Triton X-100.

One illustrative embodiment of a protocell of the invention comprises a plurality of negatively-charged, nanoporous, nanoparticulate silica cores that:

(a) are modified with an amine-containing silane selected from the group consisting of (1) a primary amine, a secondary amine a tertiary amine, each of which is functionalized with a silicon atom (2) a monoamine or a polyamine (3) N-(2-aminoethyl)-3-aminopropyltrimethoxysilane (AEPTMS) (4) 3-aminopropyltrimethoxysilane (APTMS) (5) 3-aminopropyltriethoxysilane (APTS) (6) an amino-functional trialkoxysilane, and (7) protonated secondary amines, protonated tertiary alkyl amines, protonated amidines, protonated guanidines, protonated pyridines, protonated pyrimidines, protonated pyrazines, protonated purines, protonated imidazoles, protonated pyrroles, and quaternary alkyl amines, or combinations thereof;
(b) are loaded with a siRNA or ricin toxin A-chain; and
(c) that are encapsulated by and that support a lipid bilayer comprising one of more lipids selected from the group consisting of 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-dioleoyl-sn-glycero-3-[phosphor-L-serine] (DOPS), 1,2-dioleoyl-3-trimethylammonium-propane (18:1 DOTAP), 1,2-dioleoyl-sn-glycero-3-phospho-(1′-rac-glycerol) (DOPG), 1,2-dioleoyl-sn-glycero-3-phosphoethanolamine (DOPE), 1,2-dipalmitoyl-sn-glycero-3-phosphoethanolamine (DPPE), 1,2-dioleoyl-sn-glycero-3-phosphoethanolamine-N-[methoxy(polyethylene glycol)-2000] (18:1 PEG-2000 PE), 1,2-dipalmitoyl-sn-glycero-3-phosphoethanolamine-N-[methoxy(polyethylene glycol)-2000] (16:0 PEG-2000 PE), 1-Oleoyl-2-[12-[(7-nitro-2-1,3-benzoxadiazol-4-yl)amino]lauroyl]-sn-Glycero-3-Phosphocholine (18:1-12:0 NBD PC), 1-palmitoyl-2-{12-[(7-nitro-2-1,3-benzoxadiazol-4-yl)amino]lauroyl}-sn-glycero-3-phosphocholine (16:0-12:0 NBD PC), cholesterol and mixtures/combinations thereof, and wherein the lipid bilayer comprises a cationic lipid and one or more zwitterionic phospholipids.

Protocells of the invention can comprise a wide variety of pharmaceutically-active ingredients.

The term “reporter” is used to describe an imaging agent or moiety which is incorporated into the phospholipid bilayer or cargo of protocells according to an embodiment of the present invention and provides a signal which can be measured. The moiety may provide a fluorescent signal or may be a radioisotope which allows radiation detection, among others. Exemplary fluorescent labels for use in protocells (preferably via conjugation or adsorption to the lipid bilayer or silica core, although these labels may also be incorporated into cargo elements such as DNA, RNA, polypeptides and small molecules which are delivered to cells by the protocells, include Hoechst 33342 (350/461), 4′,6-diamidino-2-phenylindole (DAPI, 356/451), Alexa Fluor® 405 carboxylic acid, succinimidyl ester (401/421), CellTracker™ Violet BMQC (415/516), CellTracker™ Green CMFDA (492/517), calcein (495/515), Alexa Fluor® 488 conjugate of annexin V (495/519), Alexa Fluor® 488 goat anti-mouse IgG (H+L) (495/519), Click-iT® AHA Alexa Fluor® 488 Protein Synthesis HCS Assay (495/519), LIVE/DEAD® Fixable Green Dead Cell Stain Kit (495/519), SYTOX® Green nucleic acid stain (504/523), MitoSOX™ Red mitochondrial superoxide indicator (510/580). Alexa Fluor® 532 carboxylic acid, succinimidyl ester (532/554), pHrodo™ succinimidyl ester (558/576), CellTracker™ Red CMTPX (577/602), Texas Red® 1,2-dihexadecanoyl-sn-glycero-3-phosphoethanolamine (Texas Red® DHPE, 583/608), Alexa Fluor® 647 hydrazide (649/666), Alexa Fluor® 647 carboxylic acid, succinimidyl ester (650/668), Ulysis™ Alexa Fluor® 647 Nucleic Acid Labeling Kit (650/670) and Alexa Fluor® 647 conjugate of annexin V (650/665). Moieties which enhance the fluorescent signal or slow the fluorescent fading may also be incorporated and include SlowFade® Gold antifade reagent (with and without DAPI) and Image-iT® FX signal enhancer. All of these are well known in the art. Additional reporters include polypeptide reporters which may be expressed by plasmids (such as histone-packaged supercoiled DNA plasmids) and include polypeptide reporters such as fluorescent green protein and fluorescent red protein. Reporters pursuant to the present invention are utilized principally in diagnostic applications including diagnosing the existence or progression of cancer (cancer tissue) in a patient and or the progress of therapy in a patient or subject.

The term “histone-packaged supercoiled plasmid DNA” is used to describe a preferred component of protocells according to the present invention which utilize a preferred plasmid DNA which has been “supercoiled” (i.e., folded in on itself using a supersaturated salt solution or other ionic solution which causes the plasmid to fold in on itself and “supercoil” in order to become more dense for efficient packaging into the protocells). The plasmid may be virtually any plasmid which expresses any number of polypeptides or encode RNA, including small hairpin RNA/shRNA or small interfering RNA/siRNA, as otherwise described herein. Once supercoiled (using the concentrated salt or other anionic solution), the supercoiled plasmid DNA is then complexed with histone proteins to produce a histone-packaged “complexed” supercoiled plasmid DNA.

“Packaged” DNA herein refers to DNA that is loaded into protocells (either adsorbed into the pores or confined directly within the nanoporous silica core itself). To minimize the DNA spatially, it is often packaged, which can be accomplished in several different ways, from adjusting the charge of the surrounding medium to creation of small complexes of the DNA with, for example, lipids, proteins, or other nanoparticles (usually, although not exclusively cationic). Packaged DNA is often achieved via lipoplexes (i.e. complexing DNA with cationic lipid mixtures). In addition, DNA has also been packaged with cationic proteins (including proteins other than histones), as well as gold nanoparticles (e.g. NanoFlares—an engineered DNA and metal complex in which the core of the nanoparticle is gold).

Any number of histone proteins, as well as other means to package the DNA into a smaller volume such as normally cationic nanoparticles, lipids, or proteins, may be used to package the supercoiled plasmid DNA “histone-packaged supercoiled plasmid DNA”, but in therapeutic aspects which relate to treating human patients, the use of human histone proteins are preferably used. In certain aspects of the invention, a combination of human histone proteins H1, H2A, H2B, H3 and H4 in a preferred ratio of 1:2:2:2:2, although other histone proteins may be used in other, similar ratios, as is known in the art or may be readily practiced pursuant to the teachings of the present invention. The DNA may also be double stranded linear DNA, instead of plasmid DNA, which also may be optionally supercoiled and/or packaged with histones or other packaging components.

Other histone proteins which may be used in this aspect of the invention include, for example, H1F, H1F0, H1FNT, H1FOO, H1FX H1H1 HIST1H1A, HIST1H1B, HIST1H1C, HIST1H1D, HIST1H1E, HIST1H1T; H2AF, H2AFB1, H2AFB2, H2AFB3, H2AFJ, H2AFV, H2AFX, H2AFY, H2AFY2, H2AFZ, H2A1, HIST1H2AA, HIST1H2AB, HIST1H2AC, HIST1H2AD, HIST1H2AE, HIST1H2AG, HIST1H2AI, HIST1H2AJ, HIST1H2AK, HIST1H2AL, HIST1H2AM, H2A2, HIST2H2AA3, HIST2H2AC, H2BF, H2BFM, HSBFS, HSBFWT, H2B1, HIST1H2BA, HIST1HSBB, HIST1HSBC, HIST1HSBD, HIST1H2BE, HIST1H2BF, HIST1H2BG, HIST1H2BH, HIST1H2BI, HIST1H2BJ, HIST1H2BK, HIST1H2BL, HIST1H2BM, HIST1H2BN, HIST1H2BO, H2B2, HIST2H2BE, H3A1, HIST1H3A, HIST1H3B, HIST1H3C, HIST1H3D, HIST1H3E, HIST1H3F, HIST1H3G, HIST1H3H, HIST1H3I, HIST1H3J, H3A2, HIST2H3C, H3A3, HIST3H3, H41, HIST1H4A, HIST1H4B, HIST1H4C, HIST1H4D, HIST1H4E, HIST1H4F, HIST1H4G, HIST1H4H, HIST1H4I, HIST1H4J, HIST1H4K, HIST1H4L, H44 and HIST4H4.

The term “nuclear localization sequence” refers to a peptide sequence incorporated or otherwise crosslinked into histone proteins which comprise the histone-packaged supercoiled plasmid DNA. In certain embodiments, protocells according to the present invention may further comprise a plasmid (often a histone-packaged supercoiled plasmid DNA) which is modified (crosslinked) with a nuclear localization sequence (note that the histone proteins may be crosslinked with the nuclear localization sequence or the plasmid itself can be modified to express a nuclear localization sequence) which enhances the ability of the histone-packaged plasmid to penetrate the nucleus of a cell and deposit its contents there (to facilitate expression and ultimately cell death. These peptide sequences assist in carrying the histone-packaged plasmid DNA and the associated histones into the nucleus of a targeted cell whereupon the plasmid will express peptides and/or nucleotides as desired to deliver therapeutic and/or diagnostic molecules (polypeptide and/or nucleotide) into the nucleus of the targeted cell. Any number of crosslinking agents, well known in the art, may be used to covalently link a nuclear localization sequence to a histone protein (often at a lysine group or other group which has a nucleophilic or electrophilic group in the side chain of the amino acid exposed pendant to the polypeptide) which can be used to introduce the histone packaged plasmid into the nucleus of a cell. Alternatively, a nucleotide sequence which expresses the nuclear localization sequence can be positioned in a plasmid in proximity to that which expresses histone protein such that the expression of the histone protein conjugated to the nuclear localization sequence will occur thus facilitating transfer of a plasmid into the nucleus of a targeted cell.

Proteins gain entry into the nucleus through the nuclear envelope. The nuclear envelope consists of concentric membranes, the outer and the inner membrane. These are the gateways to the nucleus. The envelope consists of pores or large nuclear complexes. A protein translated with a NLS will bind strongly to importin (aka karyopherin), and together, the complex will move through the nuclear pore. Any number of nuclear localization sequences may be used to introduce histone-packaged plasmid DNA into the nucleus of a cell. Preferred nuclear localization sequences include H₂N-GNQSSNFGPMKGGNFGGRSSGPYGGGGQYFAKPRNQGGYGGC-COOH SEQ I.D NO: 9, RRMKWKK (SEQ ID NO:10), PKKKRKV (SEQ ID NO: 11), and KR[PAATKKAGQA]KKKK (SEQ ID NO:12), the NLS of nucleoplasmin, a prototypical bipartite signal comprising two clusters of basic amino acids, separated by a spacer of about 10 amino acids. Numerous other nuclear localization sequences are well known in the art. See, for example, LaCasse, et al., Nuclear localization signals overlap DNA-or RNA-binding domains in nucleic acid-binding proteins. Nucl. Acids Res., 23, 1647-1656 1995); Weis, K. Importins and exportins: how to get in and out of the nucleus [published erratum appears in Trends Biochem Sci 1998 July; 23(7):235]. TIBS, 23, 185-9 (1998); and Murat Cokol, Raj Nair & Burkhard Rost, “Finding nuclear localization signals”, at the website ubic.bioc.columbia.edu/papers/2000 nls/paper.html#tab2.

The term “cancer” is used to describe a proliferation of tumor cells (neoplasms) having the unique trait of loss of normal controls, resulting in unregulated growth, lack of differentiation, local tissue invasion, and/or metastasis. As used herein, neoplasms include, without limitation, morphological irregularities in cells in tissue of a subject or host, as well as pathologic proliferation of cells in tissue of a subject, as compared with normal proliferation in the same type of tissue. Additionally, neoplasms include benign tumors and malignant tumors (e.g., colon tumors) that are either invasive or noninvasive. Malignant neoplasms are distinguished from benign neoplasms in that the former show a greater degree of dysplasia, or loss of differentiation and orientation of cells, and have the properties of invasion and metastasis. The term cancer also within context, includes drug resistant cancers, including multiple drug resistant cancers. Examples of neoplasms or neoplasias from which the target cell of the present invention may be derived include, without limitation, carcinomas (e.g., squamous-cell carcinomas, adenocarcinomas, hepatocellular carcinomas, and renal cell carcinomas), particularly those of the bladder, bone, bowel, breast, cervix, colon (colorectal), esophagus, head, kidney, liver (hepatocellular), lung, nasopharyngeal, neck, ovary, pancreas, prostate, and stomach; leukemias, such as acute myelogenous leukemia, acute lymphocytic leukemia, acute promyelocytic leukemia (APL), acute T-cell lymphoblastic leukemia, adult T-cell leukemia, basophilic leukemia, eosinophilic leukemia, granulocytic leukemia, hairy cell leukemia, leukopenic leukemia, lymphatic leukemia, lymphoblastic leukemia, lymphocytic leukemia, megakaryocytic leukemia, micromyeloblastic leukemia, monocytic leukemia, neutrophilic leukemia and stem cell leukemia; benign and malignant lymphomas, particularly Burkitt's lymphoma, Non-Hodgkin's lymphoma and B-cell lymphoma; benign and malignant melanomas; myeloproliferative diseases; sarcomas, particularly Ewing's sarcoma, hemangiosarcoma, Kaposi's sarcoma, liposarcoma, myosarcomas, peripheral neuroepithelioma, and synovial sarcoma; tumors of the central nervous system (e.g., gliomas, astrocytomas, oligodendrogliomas, ependymomas, gliobastomas, neuroblastomas, ganglioneuromas, gangliogliomas, medulloblastomas, pineal cell tumors, meningiomas, meningeal sarcomas, neurofibromas, and Schwannomas); germ-line tumors (e.g., bowel cancer, breast cancer, prostate cancer, cervical cancer, uterine cancer, lung cancer (e.g., small cell lung cancer, mixed small cell and non-small cell cancer, pleural mesothelioma, including metastatic pleural mesothelioma small cell lung cancer and non-small cell lung cancer), ovarian cancer, testicular cancer, thyroid cancer, astrocytoma, esophageal cancer, pancreatic cancer, stomach cancer, liver cancer, colon cancer, and melanoma; mixed types of neoplasias, particularly carcinosarcoma and Hodgkin's disease; and tumors of mixed origin, such as Wilms' tumor and teratocarcinomas, among others. It is noted that certain tumors including hepatocellular and cervical cancer, among others, are shown to exhibit increased levels of MET receptors specifically on cancer cells and are a principal target for compositions and therapies according to embodiments of the present invention which include a MET binding peptide complexed to the protocell.

The terms “coadminister” and “coadministration” are used synonymously to describe the administration of at least one of the protocell compositions according to the present invention in combination with at least one other agent, often at least one additional anti-cancer agent (as otherwise described herein), which are specifically disclosed herein in amounts or at concentrations which would be considered to be effective amounts at or about the same time. While it is preferred that coadministered compositions/agents be administered at the same time, agents may be administered at times such that effective concentrations of both (or more) compositions/agents appear in the patient at the same time for at least a brief period of time. Alternatively, in certain aspects of the present invention, it may be possible to have each coadministered composition/agent exhibit its inhibitory effect at different times in the patient, with the ultimate result being the inhibition and treatment of cancer, especially including hepatoccellular or cellular cancer as well as the reduction or inhibition of other disease states, conditions or complications. Of course, when more than disease state, infection or other condition is present, the present compounds may be combined with other agents to treat that other infection or disease or condition as required.

The term “anti-cancer agent” is used to describe a compound which can be formulated in combination with one or more compositions comprising protocells according to the present invention and optionally, to treat any type of cancer, in particular hepatocellular or cervical cancer, among numerous others. Anti-cancer compounds which can be formulated with compounds according to the present invention include, for example, Exemplary anti-cancer agents which may be used in the present invention include, everolimus, trabectedin, abraxane, TLK 286, AV-299, DN-101, pazopanib, GSK690693, RTA 744, ON 0910.Na, AZD 6244 (ARRY-142886), AMN-107, TKI-258, GSK461364, AZD 1152, enzastaurin, vandetanib, ARQ-197, MK-0457, MLN8054, PHA-739358, R-763, AT-9263, a FLT-3 inhibitor, a VEGFR inhibitor, an EGFR TK inhibitor, an aurora kinase inhibitor, a PIK-1 modulator, a Bcl-2 inhibitor, an HDAC inhibitor, a c-MET inhibitor, a PARP inhibitor, a Cdk inhibitor, an EGFR TK inhibitor, an IGFR-TK inhibitor, an anti-HGF antibody, a PI3 kinase inhibitors, an AKT inhibitor, a JAK/STAT inhibitor, a checkpoint-1 or 2 inhibitor, a focal adhesion kinase inhibitor, a Map kinase kinase (mek) inhibitor, a VEGF trap antibody, pemetrexed, erlotinib, dasatanib, nilotinib, decatanib, panitumumab, amrubicin, oregovomab, Lep-etu, nolatrexed, azd2171, batabulin, ofatumumab, zanolimumab, edotecarin, tetrandrine, rubitecan, tesmilifene, oblimersen, ticilimumab, ipilimumab, gossypol, Bio 111, 131-I-TM-601, ALT-110, BIO 140, CC 8490, cilengitide, gimatecan, IL13-PE38QQR, INO 1001, IPdR₁ KRX-0402, lucanthone, LY 317615, neuradiab, vitespan, Rta 744, Sdx 102, talampanel, atrasentan, Xr 311, romidepsin, ADS-100380, sunitinib, 5-fluorouracil, vorinostat, etoposide, gemcitabine, doxorubicin, liposomal doxorubicin, 5′-deoxy-5-fluorouridine, vincristine, temozolomide, ZK-304709, seliciclib; PD0325901, AZD-6244, capecitabine, L-Glutamic acid, N-[4-[2-(2-amino-4,7-dihydro-4-oxo-1H-pyrrolo[2,3-d]pyrimidin-5-yl)ethyl]benzoyl]-, disodium salt, heptahydrate, camptothecin, PEG-labeled irinotecan, tamoxifen, toremifene citrate, anastrazole, exemestane, letrozole, DES (diethylstilbestrol), estradiol, estrogen, conjugated estrogen, bevacizumab, IMC-1C11, CHIR-258,); 3-[5-(methylsulfonylpiperadinemethyl)-indolylj-quinolone, vatalanib, AG-013736, AVE-0005, the acetate salt of [D-Ser(Bu t) 6,Azgly 10] (pyro-Glu-His-Trp-Ser-Tyr-D-Ser(Bu t)-Leu-Arg-Pro-Azgly-NH₂ acetate [C₅₉H₈₄N₁₈Oi₄-(C₂H₄O₂)_{X} where x=1 to 2.4], goserelin acetate, leuprolide acetate, triptorelin pamoate, medroxyprogesterone acetate, hydroxyprogesterone caproate, megestrol acetate, raloxifene, bicalutamide, flutamide, nilutamide, megestrol acetate, CP-724714; TAK-165, HKI-272, erlotinib, lapatanib, canertinib, ABX-EGF antibody, erbitux, EKB-569, PKI-166, GW-572016, lonafamib, BMS-214662, tipifarnib; amifostine, NVP-LAQ824, suberoyl analide hydroxamic acid, valproic acid, trichostatin A, FK-228, SU11248, sorafenib, KRN951, aminoglutethimide, amsacrine, anagrelide, L-asparaginase, Bacillus Calmette-Guerin (BCG) vaccine, bleomycin, buserelin, busulfan, carboplatin, carmustine, chlorambucil, cisplatin, cladribine, clodronate, cyproterone, cytarabine, dacarbazine, dactinomycin, daunorubicin, diethylstilbestrol, epirubicin, fludarabine, fludrocortisone, fluoxymesterone, flutamide, gemcitabine, gleevac, hydroxyurea, idarubicin, ifosfamide, imatinib, leuprolide, levamisole, lomustine, mechlorethamine, melphalan, 6-mercaptopurine, mesna, methotrexate, mitomycin, mitotane, mitoxantrone, nilutamide, octreotide, oxaliplatin, pamidronate, pentostatin, plicamycin, porfimer, procarbazine, raltitrexed, rituximab, streptozocin, teniposide, testosterone, thalidomide, thioguanine, thiotepa, tretinoin, vindesine, 13-cis-retinoic acid, phenylalanine mustard, uracil mustard, estramustine, altretamine, floxuridine, 5-deooxyuridine, cytosine arabinoside, 6-mecaptopurine, deoxycoformycin, calcitriol, valrubicin, mithramycin, vinblastine, vinorelbine, topotecan, razoxin, marimastat, COL-3, neovastat, BMS-275291, squalamine, endostatin, SU5416, SU6668, EMD121974, interleukin-12, IM862, angiostatin, vitaxin, droloxifene, idoxyfene, spironolactone, finasteride, cimitidine, trastuzumab, denileukin diftitox, gefitinib, bortezimib, paclitaxel, cremophor-free paclitaxel, docetaxel, epithilone B, BMS-247550, BMS-310705, droloxifene, 4-hydroxytamoxifen, pipendoxifene, ERA-923, arzoxifene, fulvestrant, acolbifene, lasofoxifene, idoxifene, TSE-424, HMR-3339, ZK186619, topotecan, PTK787/ZK 222584, VX-745, PD 184352, rapamycin, 40-O-(2-hydroxyethyl)-rapamycin, temsirolimus, AP-23573, RAD001, ABT-578, BC-210, LY294002, LY292223, LY292696, LY293684, LY293646, wortmannin, ZM336372, L-779,450, PEG-filgrastim, darbepoetin, erythropoietin, granulocyte colony-stimulating factor, zolendronate, prednisone, cetuximab, granulocyte macrophage colony-stimulating factor, histrelin, pegylated interferon alfa-2a, interferon alfa-2a, pegylated interferon alfa-2b, interferon alfa-2b, azacitidine, PEG-L-asparaginase, lenalidomide, gemtuzumab, hydrocortisone, interleukin-11, dexrazoxane, alemtuzumab, all-transretinoic acid, ketoconazole, interleukin-2, megestrol, immune globulin, nitrogen mustard, methylprednisolone, ibritgumomab tiuxetan, androgens, decitabine, hexamethylmelamine, bexarotene, tositumomab, arsenic trioxide, cortisone, editronate, mitotane, cyclosporine, liposomal daunorubicin, Edwina-asparaginase, strontium 89, casopitant, netupitant, an NK-1 receptor antagonists, palonosetron, aprepitant, diphenhydramine, hydroxyzine, metoclopramide, lorazepam, alprazolam, haloperidol, droperidol, dronabinol, dexamethasone, methylprednisolone, prochlorperazine, granisetron, ondansetron, dolasetron, tropisetron, pegfilgrastim, erythropoietin, epoetin alfa, darbepoetin alfa and mixtures thereof.

The term “antihepatocellular cancer agent” is used throughout the specification to describe an anticancer agent which may be used to inhibit, treat or reduce the likelihood of hepatocellular cancer, or the metastasis of that cancer. Anticancer agents which may find use in the present invention include for example, nexavar (sorafenib), sunitinib, bevacizumab, tarceva (erlotinib), tykerb (lapatinib) and mixtures thereof. In addition, other anticancer agents may also be used in the present invention, where such agents are found to inhibit metastasis of cancer, in particular, hepatocellular cancer.

The term “antiviral agent” is used to describe a bioactive agent/drug which inhibits the growth and/or elaboration of a virus, including mutant strains such as drug resistant viral strains. Preferred antiviral agents include anti-HIV agents, anti-HBV agents and anti-HCV agents. In certain aspects of the invention, especially where the treatment of hepatocellular cancer is the object of therapy, the inclusion of an anti-hepatitis C agent or anti-hepatitis B agent may be combined with other traditional anticancer agents to effect therapy, given that hepatitis B virus (HBV) and/or hepatitis C virus (HCV) is often found as a primary or secondary infection or disease state associated with hepatocellular cancer. Anti-HBV agents which may be used in the present invention, either as a cargo component in the protocell or as an additional bioactive agent in a pharmaceutical composition which includes a population of protocells includes such agents as Hepsera (adefovir dipivoxil), lamivudine, entecavir, telbivudine, tenofovir, emtricitabine, clevudine, valtoricitabine, amdoxovir, pradefovir, racivir, BAM 205, nitazoxanide, UT 231-B, Bay 41-4109, EHT899, zadaxin (thymosin alpha-1) and mixtures thereof. Typical anti-HCV agents for use in the invention include such agents as boceprevir, daclatasvir, asunapavir, INX-189, FV-100, NM 283, VX-950 (telaprevir), SCH 50304, TMC435, VX-500, BX-813, SCH503034, R1626, ITMN-191 (R7227), R7128, PF-868554, TT033, CGH-759, GI 5005, MK-7009, SIRNA-034, MK-0608, A-837093, GS 9190, GS 9256, GS 9451, GS 5885, GS 6620, GS 9620, GS9669, ACH-1095, ACH-2928, GSK625433, TG4040 (MVA-HCV), A-831, F351, NS5A, NS4B, ANA598, A-689, GNI-104, IDX102, ADX184, ALS-2200, ALS-2158, BI 201335, BI 207127, BIT-225, BIT-8020, GL59728, GL60667, PSI-938, PSI-7977, PSI-7851, SCY-635, ribavirin, pegylated interferon, PHX1766, SP-30 and mixtures thereof.

The term “anti-HIV agent” refers to a compound which inhibits the growth and/or elaboration of HIV virus (I and/or II) or a mutant strain thereof. Exemplary anti-HIV agents for use in the present invention which can be included as cargo in protocells according to the present invention include, for example, including nucleoside reverse transcriptase inhibitors (NRTI), other non-nucloeoside reverse transcriptase inhibitors (i.e., those which are not representative of the present invention), protease inhibitors, fusion inhibitors, among others, exemplary compounds of which may include, for example, 3TC (Lamivudine), AZT (Zidovudine), (−)-FTC, ddl (Didanosine), ddC (zalcitabine), abacavir (ABC), tenofovir (PMPA), D-D4FC (Reverset), D4T (Stavudine), Racivir, L-FddC, L-FD4C, NVP (Nevirapine), DLV (Delavirdine), EFV (Efavirenz), SQVM (Saquinavir mesylate), RTV (Ritonavir), IDV (Indinavir), SQV (Saquinavir), NFV (Nelfinavir), APV (Amprenavir), LPV (Lopinavir), fusion inhibitors such as T20, among others, fuseon and mixtures thereof

The term “targeting active species” is used to describe a compound or moiety which is complexed or preferably covalently bonded to the surface of a protocell according to the present invention which binds to a moiety on the surface of a cell to be targeted so that the protocell may selectively bind to the surface of the targeted cell and deposit its contents into the cell. The targeting active species for use in the present invention is preferably a targeting peptide as otherwise described herein, a polypeptide including an antibody or antibody fragment, an aptamer, or a carbohydrate, among other species which bind to a targeted cell.

The term “targeting peptide” is used to describe a preferred targeting active species which is a peptide of a particular sequence which binds to a receptor or other polypeptide in cancer cells and allows the targeting of protocells according to the present invention to particular cells which express a peptide (be it a receptor or other functional polypeptide) to which the targeting peptide binds. In the present invention, exemplary targeting peptides include, for example, SP94 free peptide (H₂N-SFSIILTPILPL-COOH, SEQ ID NO: 6), SP94 peptide modified with a C-terminal cysteine for conjugation with a crosslinking agent (H₂N-GLFHAIAHFIHGGWHGLIHGWYGGC-COOH (SEQ ID. NO: 13) or an 8 mer polyarginine (H₂N-RRRRRRRR-COOH, SEQ ID NO:14), a modified SP94 peptide (H₂N-SFSIILTPILPLEEEGGC-COOH, SEQ ID NO: 8) or a MET binding peptide as otherwise disclosed herein. Other targeting peptides are known in the art. Targeting peptides may be complexed or preferably, covalently linked to the lipid bilayer through use of a crosslinking agent as otherwise described herein.

The terms “fusogenic peptide” and “endosomolytic peptide” are used synonymously to describe a peptide which is optionally and preferred crosslinked onto the lipid bilayer surface of the protocells according to the present invention. Fusogenic peptides are incorporated onto protocells in order to facilitate or assist escape from endosomal bodies and to facilitate the introduction of protocells into targeted cells to effect an intended result (therapeutic and/or diagnostic as otherwise described herein). Representative and preferred fusogenic peptides for use in protocells according to the present invention include H5WYG peptide, H₂N-GLFHAIAHFIHGGWHGLIHGWYGGC-COOH (SEQ ID. NO: 13) or an 8 mer polyarginine (H₂N-RRRRRRRR-COOH, SEQ ID NO:14), among others known in the art.

The term “crosslinking agent” is used to describe a bifunctional compound of varying length containing two different functional groups which may be used to covalently link various components according to the present invention to each other. Crosslinking agents according to the present invention may contain two electrophilic groups (to react with nucleophilic groups on peptides of oligonucleotides, one electrophilic group and one nucleophilic group or two nucleophilic groups). The crosslinking agents may vary in length depending upon the components to be linked and the relative flexibility required. Crosslinking agents are used to anchor targeting and/or fusogenic peptides to the phospholipid bilayer, to link nuclear localization sequences to histone proteins for packaging supercoiled plasmid DNA and in certain instances, to crosslink lipids in the lipid bilayer of the protocells as well as conjugate CD47 molecules to lipids which are incorporated into the lipid bilayer of the protocell. There are a large number of crosslinking agents which may be used in the present invention, many commercially available or available in the literature. Preferred crosslinking agents for use in the present invention include, for example, 1-Ethyl-3-[3-dimethylaminopropyl]carbodiimide hydrochloride (EDC), succinimidyl 4-[N-maleimidomethyl]cyclohexane-1-carboxylate (SMCC), N-[13-Maleimidopropionic acid] hydrazide (BMPH), NHS-(PEG)ₙ-maleimide, succinimidyl-[(N-maleimidopropionamido)-tetracosaethyleneglycol] ester (SM(PEG)₂₄), and succinimidyl 6-[3′-(2-pyridyldithio)-propionamido] hexanoate (LC-SPDP), among others.

As discussed in detail above, the porous nanoparticle core of the present invention can include porous nanoparticles having at least one dimension, for example, a width or a diameter of about 3000 nm or less, about 1000 nm or less, about 500 nm or less, about 200 nm or less. Preferably, the nanoparticle core is spherical with a preferred diameter of about 500 nm or less, more preferably about 8-10 nm to about 200 nm. In embodiments, the porous particle core can have various cross-sectional shapes including a circular, rectangular, square, or any other shape. In certain embodiments, the porous particle core can have pores with a mean pore size ranging from about 2 nm to about 30 nm, although the mean pore size and other properties (e.g., porosity of the porous particle core) are not limited in accordance with various embodiments of the present teachings.

In general, protocells according to the present invention are biocompatible. Drugs and other cargo components are often loaded by adsorption and/or capillary filling of the pores of the particle core up to approximately 50% by weight of the final protocell (containing all components). In certain embodiments according to the present invention, the loaded cargo can be released from the porous surface of the particle core (mesopores), wherein the release profile can be determined or adjusted by, for example, the pore size, the surface chemistry of the porous particle core, the pH value of the system, and/or the interaction of the porous particle core with the surrounding lipid bilayer(s) as generally described herein.

In the present invention, the porous nanoparticle core used to prepare the protocells can be tuned in to be hydrophilic or progressively more hydrophobic as otherwise described herein and can be further treated to provide a more hydrophilic surface. For example, mesoporous silica particles can be further treated with ammonium hydroxide and hydrogen peroxide to provide a higher hydrophilicity. In preferred aspects of the invention, the lipid bilayer is fused onto the porous particle core to form the protocell. Protocells according to the present invention can include various lipids in various weight ratios, preferably including 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-dioleoyl-sn-glycero-3-[phosphor-L-serine] (DOPS), 1,2-dioleoyl-3-trimethylammonium-propane (18:1 DOTAP), 1,2-dioleoyl-sn-glycero-3-phospho-(1′-rac-glycerol) (DOPG), 1,2-dioleoyl-sn-glycero-3-phosphoethanolamine (DOPE), 1,2-dipalmitoyl-sn-glycero-3-phosphoethanolamine (DPPE), 1,2-dioleoyl-sn-glycero-3-phosphoethanolamine-N-[methoxy(polyethylene glycol)-2000] (18:1 PEG-2000 PE), 1,2-dipalmitoyl-sn-glycero-3-phosphoethanolamine-N-[methoxy(polyethylene glycol)-2000] (16:0 PEG-2000 PE), 1-Oleoyl-2-[12-[(7-nitro-2-1,3-benzoxadiazol-4-yl)amino]lauroyl]-sn-Glycero-3-Phosphocholine (18:1-12:0 NBD PC), 1-palmitoyl-2-{12-[(7-nitro-2-1,3-benzoxadiazol-4-yl)amino]lauroyl}-sn-glycero-3-phosphocholine (16:0-12:0 NBD PC), cholesterol and mixtures/combinations thereof. One or more of the lipids may be modified to contain a CD47 molecule which can be incorporated into the lipid bilayer of the protocell. Alternatively, the lipid bilayer may be mixed with a portion of cellular plasma membrane which contains CD47 and the mixture may be fused onto a protocell resulting in a protocell containing a lipid bilayer which also contains CD47 molecules.

The lipid bilayer which is used to prepare protocells according to the present invention can be prepared, for example, by extrusion of hydrated lipid films through a filter with pore size of, for example, about 100 nm, using standard protocols known in the art or as otherwise described herein. The filtered lipid bilayer films can then be fused with the porous particle cores, for example, by pipette mixing. In certain embodiments, excess amount of lipid bilayer or lipid bilayer films can be used to form the protocell in order to improve the protocell colloidal stability.

In certain diagnostic embodiments, various dyes or fluorescent (reporter) molecules can be included in the protocell cargo (as expressed by as plasmid DNA) or attached to the porous particle core and/or the lipid bilayer for diagnostic purposes. For example, the porous particle core can be a silica core or the lipid bilayer and can be covalently labeled with FITC (green fluorescence), while the lipid bilayer or the particle core can be covalently labeled with FITC Texas red (red fluorescence). The porous particle core, the lipid bilayer and the formed protocell can then be observed by, for example, confocal fluorescence for use in diagnostic applications. In addition, as discussed herein, plasmid DNA can be used as cargo in protocells according to the present invention such that the plasmid may express one or more fluorescent proteins such as fluorescent green protein or fluorescent red protein which may be used in diagnostic applications.

In various embodiments, the protocell is used in a synergistic system where the lipid bilayer fusion or liposome fusion (i.e., on the porous particle core) is loaded and sealed with various cargo components with the pores (mesopores) of the particle core, thus creating a loaded protocell useful for cargo delivery across the cell membrane of the lipid bilayer or through dissolution of the porous nanoparticle, if applicable. In certain embodiments, in addition to fusing a single lipid (e.g., phospholipids) bilayer, multiple bilayers with opposite charges can be successively fused onto the porous particle core to further influence cargo loading and/or sealing as well as the release characteristics of the final protocell

A fusion and synergistic loading mechanism can be included for cargo delivery. For example, cargo can be loaded, encapsulated, or sealed, synergistically through liposome fusion on the porous particles. The cargo can include, for example, small molecule drugs (e.g. especially including anticancer drugs and/or antiviral drugs such as anti-HBV or anti-HCV drugs), peptides, proteins, antibodies, DNA (especially plasmid DNA, including the preferred histone-packaged super coiled plasmid DNA), RNAs (including shRNA and siRNA (which may also be expressed by the plasmid DNA incorporated as cargo within the protocells) fluorescent dyes, including fluorescent dye peptides which may be expressed by the plasmid DNA incorporated within the protocell.

In embodiments according to the present invention, the cargo can be loaded into the pores (mesopores) of the porous particle cores to form the loaded protocell. In various embodiments, any conventional technology that is developed for liposome-based drug delivery, for example, targeted delivery using PEGylation, can be transferred and applied to the protocells of the present invention.

As discussed above, electrostatics and pore size can play a role in cargo loading. For example, porous silica nanoparticles can carry a negative charge and the pore size can be tunable from about 2 nm to about 10 nm or more. Negatively charged nanoparticles can have a natural tendency to adsorb positively charged molecules and positively charged nanoparticles can have a natural tendency to adsorb negatively charged molecules. In various embodiments, other properties such as surface wettability (e.g., hydrophobicity) can also affect loading cargo with different hydrophobicity.

In various embodiments, the cargo loading can be a synergistic lipid-assisted loading by tuning the lipid composition. For example, if the cargo component is a negatively charged molecule, the cargo loading into a negatively charged silica can be achieved by the lipid-assisted loading. In certain embodiments, for example, a negatively species can be loaded as cargo into the pores of a negatively charged silica particle when the lipid bilayer is fused onto the silica surface showing a fusion and synergistic loading mechanism. In this manner, fusion of a non-negatively charged (i.e., positively charged or neutral) lipid bilayer or liposome on a negatively charged mesoporous particle can serve to load the particle core with negatively charged cargo components. The negatively charged cargo components can be concentrated in the loaded protocell having a concentration exceed about 100 times as compared with the charged cargo components in a solution. In other embodiments, by varying the charge of the mesoporous particle and the lipid bilayer, positively charged cargo components can be readily loaded into protocells.

Once produced, the loaded protocells can have a cellular uptake for cargo delivery into a desirable site after administration. For example, the cargo-loaded protocells can be administered to a patient or subject and the protocell comprising a targeting peptide can bind to a target cell and be internalized or uptaken by the target cell, for example, a cancer cell in a subject or patient. Due to the internalization of the cargo-loaded protocells in the target cell, cargo components can then be delivered into the target cells. In certain embodiments the cargo is a small molecule, which can be delivered directly into the target cell for therapy. In other embodiments, negatively charged DNA or RNA (including shRNA or siRNA), especially including a DNA plasmid which is preferably formulated as histone-packaged supercoiled plasmid DNA preferably modified with a nuclear localization sequence can be directly delivered or internalized by the targeted cells. Thus, the DNA or RNA can be loaded first into a protocell and then into then through the target cells through the internalization of the loaded protocells.

As discussed, the cargo loaded into and delivered by the protocell to targeted cells includes small molecules or drugs (especially anti-cancer or anti-HBV and/or anti-HCV agents), bioactive macromolecules (bioactive polypeptides such as ricin toxin A-chain or diphtheria toxin A-chain or RNA molecules such as shRNA and/or siRNA as otherwise described herein) or histone-packaged supercoiled plasmid DNA which can express a therapeutic or diagnostic peptide or a therapeutic RNA molecule such as shRNA or siRNA, wherein the histone-packaged supercoiled plasmid DNA is optionally and preferably modified with a nuclear localization sequence which can localize and concentrate the delivered plasmid DNA into the nucleus of the target cell. As such, loaded protocells can deliver their cargo into targeted cells for therapy or diagnostics.

In various embodiments according to the present invention, the protocells and/or the loaded protocells can provide a targeted delivery methodology for selectively delivering the protocells or the cargo components to targeted cells (e.g., cancer cells). For example, a surface of the lipid bilayer can be modified by a targeting active species that corresponds to the targeted cell. The targeting active species may be a targeting peptide as otherwise described herein, a polypeptide including an antibody or antibody fragment, an aptamer, a carbohydrate or other moiety which binds to a targeted cell. In preferred aspects of the invention, the targeting active species is a targeting peptide as otherwise described herein. In certain embodiments, preferred peptide targeting species include a MET binding peptide as otherwise described herein.

For example, by providing a targeting active species (preferably, a targeting peptide) on the surface of the loaded protocell, the protocell selectively binds to the targeted cell in accordance with the present teachings. In one embodiment, by conjugating an exemplary targeting peptide SP94 or analog or a MET binding peptide as otherwise described herein that targets cancer cells, including cancer liver cells to the lipid bilayer, a large number of the cargo-loaded protocells can be recognized and internalized by this specific cancer cells due to the specific targeting of the exemplary SP94 or MET binding peptide with the cancer (including liver) cells. In most instances, if the protocells are conjugated with the targeting peptide, the protocells will selectively bind to the cancer cells and no appreciable binding to the non-cancerous cells occurs.

Once bound and taken up by the target cells, the loaded protocells can release cargo components from the porous particle and transport the released cargo components into the target cell. For example, sealed within the protocell by the liposome fused bilayer on the porous particle core, the cargo components can be released from the pores of the lipid bilayer, transported across the protocell membrane of the lipid bilayer and delivered within the targeted cell. In embodiments according to the present invention, the release profile of cargo components in protocells can be more controllable as compared with when only using liposomes as known in the prior art. The cargo release can be determined by, for example, interactions between the porous core and the lipid bilayer and/or other parameters such as pH value of the system. For example, the release of cargo can be achieved through the lipid bilayer, through dissolution of the porous silica; while the release of the cargo from the protocells can be pH-dependent.

In certain embodiments, the pH value for cargo is often less than 7, preferably about 4.5 to about 6.0, but can be about pH 14 or less. Lower pHs tend to facilitate the release of the cargo components significantly more than compared with high pHs. Lower pHs tend to be advantageous because the endosomal compartments inside most cells are at low pHs (about 5.5), but the rate of delivery of cargo at the cell can be influenced by the pH of the cargo. Depending upon the cargo and the pH at which the cargo is released from the protocell, the release of cargo can be relative short (a few hours to a day or so) or span for several days to about 20-30 days or longer. Thus, the present invention may accommodate immediate release and/or sustained release applications from the protocells themselves.

In certain embodiments, the inclusion of surfactants can be provided to rapidly rupture the lipid bilayer, transporting the cargo components across the lipid bilayer of the protocell as well as the targeted cell. In certain embodiments, the phospholipid bilayer of the protocells can be ruptured by the application/release of a surfactant such as sodium dodecyl sulfate (SDS), among others to facilitate a rapid release of cargo from the protocell into the targeted cell. Other than surfactants, other materials can be included to rapidly rupture the bilayer. One example would be gold or magnetic nanoparticles that could use light or heat to generate heat thereby rupturing the bilayer. Additionally, the bilayer can be tuned to rupture in the presence of discrete biophysical phenomena, such as during inflammation in response to increased reactive oxygen species production. In certain embodiments, the rupture of the lipid bilayer can in turn induce immediate and complete release of the cargo components from the pores of the particle core of the protocells. In this manner, the protocell platform can provide an increasingly versatile delivery system as compared with other delivery systems in the art. For example, when compared to delivery systems using nanoparticles only, the disclosed protocell platform can provide a simple system and can take advantage of the low toxicity and immunogenicity of liposomes or lipid bilayers along with their ability to be PEGylated or to be conjugated to extend circulation time and effect targeting. In another example, when compared to delivery systems using liposome only, the protocell platform can provide a more stable system and can take advantage of the mesoporous core to control the loading and/or release profile and provide increased cargo capacity.

In addition, the lipid bilayer and its fusion on porous particle core can be fine-tuned to control the loading, release, and targeting profiles and can further comprise fusogenic peptides and related peptides to facilitate delivery of the protocells for greater therapeutic and/or diagnostic effect. Further, the lipid bilayer of the protocells can provide a fluidic interface for ligand display and multivalent targeting, which allows specific targeting with relatively low surface ligand density due to the capability of ligand reorganization on the fluidic lipid interface. Furthermore, the disclosed protocells can readily enter targeted cells while empty liposomes without the support of porous particles cannot be internalized by the cells.

Embodiments of the present invention are directed to protocells for specific targeting of cells within a patient's body, including cancer cells and infected cells which comprise a 1) a nanoporous silica or metal oxide core; 2) a supported lipid bilayer which comprises an effective amount of CD47 or an active fragment thereof (“CD47 molecule”); 3) at least one agent which preferably facilitates cancer cell death or treats a bacterial or viral infection (such as a traditional small molecule, a macromolecular cargo such as a polynucleotide such as RNA, DNA and/or a polypeptide, protein or carbohydrate (e.g. siRNA, shRNA other micro RNA, a protein toxin such as ricin toxin A-chain or diphtheria toxin A-chain, double stranded or linear DNA, plasmid DNA which may be supercoiled and/or packaged such as with histones and disposed within the nanoporous silica core which may be supercoiled in order to more efficiently package the DNA into protocells) which is optionally modified with a nuclear localization sequence to assist in localizing protocells within the nucleus of the cancer cell and the ability to express peptides involved in therapy (apoptosis/cell death) of the cancer cell or as a reporter, a targeting peptide which targets cancer or other infected (virus, bacterial) cells in tissue to be treated such that binding of the protocell to the targeted cells is specific and enhanced and a fusogenic peptide that promotes endosomal escape of protocells and encapsulated cargo, especially including polynucleotides such as RNA and DNA. Protocells according to the present invention may be used to treat cancer, bacterial and viral infections, by selectively binding to tissue or to function in diagnosis of cancer, bacterial and viral infections, including cancer treatment and drug discovery.

In certain embodiments, protocells of the invention facilitate the delivery of a wide variety of active ingredients with reduced interference by the immune system because of the incorporation of CD47 molecules on the surface of the protocell. Significantly, these protocells effectively enhance delivery of active ingredients including macromolecules via numerous routes of administration.

In another embodiment, the invention provides stable, hydrophobic and super-hydrophobic porous nanoparticles useful in the delivery of a wide variety of active ingredients in environments such as the stomach (oral dosage) and other dosage forms.

In certain other embodiments, the invention provides transdermal protocells that are useful in delivering a wide-variety of active ingredients, protocells comprising a plurality of mesoporous, nanoparticulate silica cores that are loaded with a siRNA that induces sequence-specific degradation of mRNA, and gastrically-buoyant protocells that enable delivery of a wide variety of active ingredients in the stomach. The inclusion of CD47 molecules on the surface of the protocells facilitates biodistribution and minimizes interference from the patient's immune system.

In one embodiment, the present invention is directed to a protocell comprising a nanoporous silica or metal oxide core with a supported lipid bilayer comprising at least one CD47 molecule and preferably an effective amount of CD47 which allows the protocell to avoid impact by a patient's immune system for enhanced bioavailability. The CD47 molecule may be conjugated to the lipid bilayer by way of any number of conjugating moieties (e.g. crosslinking agents as described herein) or may be introduced into the bilayer by incorporating a certain weight percentage of cellular plasma membrane (which contains CD47, such as the cell membrane of erythrocytes) into the fused lipid bilayer of the protocell. In this embodiment, the fused lipid bilayer comprises between about 0.5% to about 99.5% by weight, about 1% and 99% by weight, about 5% and about 95% by weight, about 10% and about 90% by weight, about 15% and about 85% by weight, about 20% and about 80% by weight, about 25% and about 75% by weight, about 30% and about 70% by weight, about 35% and about 65% by weight, about 40% and about 60% by weight, about 45% and about 55% by weight and about 50% by weight of a synthetic lipid bilayer and about 0.5% to about 99.5% by weight, about 1% and 99% by weight, about 5% and about 95% by weight, about 10% and about 90% by weight, about 15% and about 85% by weight, about 20% and about 80% by weight, about 25% and about 75% by weight, about 30% and about 70% by weight, about 35% and about 65% by weight, about 40% and about 60% by weight, about 45% and about 55% by weight and about 50% by weight of a cellular plasma membrane. It is noted that the amount of cellular plasma membrane incorporated into the fused bilayer of the protocell is sufficient to substantially reduce the effect of immune system on the ability of the protocell to biodistribute in a patient after administration.

In certain aspects, the present invention is directed to a cell-targeting porous protocell comprising a nanoporous silica or metal oxide core with a supported lipid bilayer comprising CD47 molecules (whether conjugated to lipids within the lipid bilayer or from the incorporation of cellular plasma membrane in combination with other lipids, including autologous cellular plasma membrane into the lipid bilayer of the protocell), and at least one further component selected from the group consisting of
a cell targeting species;
a fusogenic peptide that promotes endosomal escape of protocells and encapsulated DNA;
cargo comprising at least one cargo component selected from the group consisting of polynucleotides (RNA and/or DNA) including double stranded linear DNA or plasmid DNA, small interfering RNA, small hairpin RNA, microRNA, antisense RNA, polypeptides, proteins, or a mixture thereof;
cargo comprising a drug or other small molecule;
an imaging agent,
wherein one of said cargo components is optionally conjugated further with a nuclear localization sequence.

In certain embodiments, protocells according to embodiments of the invention comprise a nanoporous silica core with a supported lipid bilayer comprising an effective number of CD47 molecules; a cargo comprising at least one therapeutic agent which optionally treats bacterial and/or viral infected cells or facilitates cancer cell death such as a traditional small molecule, a macromolecular cargo (e.g. siRNA such as 5565, 57824 and/or s10234, among others, shRNA or a protein toxin such as a ricin toxin A-chain or diphtheria toxin A-chain) and/or a packaged plasmid DNA (in certain embodiments—histone packaged) disposed within the nanoporous silica core (preferably supercoiled as otherwise described herein in order to more efficiently package the DNA into protocells as a cargo element) which is optionally modified with a nuclear localization sequence to assist in localizing/presenting the plasmid within the nucleus of the cancer cell and the ability to express peptides involved in therapy (e.g., apoptosis/cell death of the cancer cell) or as a reporter (fluorescent green protein, fluorescent red protein, among others, as otherwise described herein) for diagnostic applications. Protocells according to the present invention include a targeting peptide which targets cells for therapy (e.g., infected cells and/or cancer cells in tissue to be treated) such that binding of the protocell to the targeted cells is specific and enhanced and a fusogenic peptide that promotes endosomal escape of protocells and encapsulated DNA. Protocells according to the present invention may be used in therapy or diagnostics, more specifically to treat cancer and other diseases and chronic conditions, including viral infections, especially including hepatocellular (liver) cancer. In other aspects of the invention, protocells use binding peptides (e.g., MET binding peptides) which selectively bind to cancer tissue (including hepatocellular, ovarian and cervical cancer tissue, among other tissue) for therapy and/or diagnosis of cancer, including the monitoring of cancer treatment and drug discovery.

In one aspect, protocells according to embodiments of the present invention comprise a porous nanoparticle protocell which often comprises a nanoporous silica core with a supported lipid bilayer comprising CD47 molecules. In this aspect of the invention, the protocell comprises a targeting peptide which can be a MET receptor binding, which can also be in combination with a fusogenic peptide on the surface of the protocell. The protocell may be loaded with various therapeutic and/or diagnostic cargo, including for example, small molecules (therapeutic and/or diagnostic, especially including anticancer and/or antiviral agents (for treatment of HBV and/or HCV), macromolecules including polypeptides and nucleotides, including RNA (shRNA and siRNA) or plasmid DNA which may be supercoiled and histone-packaged including a nuclear localization sequence, which may be therapeutic and/or diagnostic (including a reporter molecule such as a fluorescent peptide, including fluorescent green protein/FGP, fluorescent red protein/FRP, among others).

Transdermal embodiments of the invention include protocells comprised of porous nanoparticulates that (a) are loaded with one or more pharmaceutically-active agents and (b) that are encapsulated by and that support a lipid bilayer comprising at least one CD47 molecule, preferably a population of such molecules either conjugated to the lipids in the bilayer or from cellular plasma membrane which has been mixed with other lipids and fused as a bilayer to the nanoparticle, wherein the lipid bilayer further comprises one or more stratum corneum permeability-enhancers selected form the group consisting of monounsaturated omega-9 fatty acids (oleic acid, elaidic acid, eicosenoic acid, mead acid, erucic acid, and nervonic acid, most preferably oleic acid), an alcohol, a diol (most preferably polyethylene glycol (PEG)), R8 peptide, and edge activators such as bile salts, polyoxyethylene esters and polyoxyethylene ethers, a single-chain surfactant (e.g. sodium deoxycholate), and wherein the protocell has an average diameter of between about 25 nm to about 300 nm, more preferably between about 30 nm to about 250 nm, more preferably between about 30 nm to about 240 nm, more preferably between about 30 nm to about 210 nm, more preferably between about 30 nm to about 190 nm, more preferably between about 30 nm to about 160 nm, more preferably between about 30 nm to about 130 nm, more preferably between about 30 nm to about 100 nm, more preferably between about 30 nm to about 90 nm, more preferably between about 30 nm to about 80 nm, more preferably between about 65 nm to about 75 nm, more preferably between about 65 nm to about 66, 67, 68, 69, 70, 71, 72, 73, 74 or 75 nm, most preferably around 70 nm.

Thus, in some embodiments there is a transdermal protocell comprising a plurality of porous nanoparticulates that (a) are loaded with one or more pharmaceutically-active agents and (b) that are encapsulated by and that support a lipid bilayer comprising CD47 molecules, wherein the lipid bilayer comprises one or more stratum corneum permeability-enhancers selected from the group consisting of a monounsaturated omega-9 fatty acid, an alcohol, a diol, a solvent, a co-solvent, permeation promoting peptides and nucleotides, and an edge activator, wherein the protocell has an average diameter of between about 50 nm to about 300 nm. The monounsaturated omega-9 fatty acid can be selected from the group consisting of oleic acid, elaidic acid, eicosenoic acid, mead acid, erucic acid, and nervonic acid, most preferably oleic acid, and mixtures thereof. The alcohol can be selected from the group consisting of methanol, ethanol, propanol, and butanol, and mixtures thereof, and the solvent and co-solvent are selected from the group consisting of PEG 400 and DMSO. The diol can be selected from the group consisting of ethylene glycol and polyethylene glycol, and mixtures thereof. The edge activator can be selected from the group consisting of bile salts, polyoxyethylene esters and polyoxyethylene ethers, and a single-chain surfactant, and mixtures thereof. In a preferred embodiment, the edge activator is sodium deoxycholate.

The transdermal route of administration can be a superior route in comparison to the oral and parenteral routes, depending on the embodiment and therapeutic or diagnostic agent to be delivered. Orally administered drugs are subject to first-pass metabolism, and can have adverse interactions with food and the broad pH-range of the digestive tract. Parenteral administration is painful, generates bio-hazardous waste, and cannot be self-administered. Transdermal drug delivery addresses all of the fore-mentioned issues associated with both the oral and parenteral routes. Additionally, transdermal delivery systems (TDDS) allow for a controlled release profile that is sustained over several days. However, the main challenge associated with transdermal drug delivery lies in the skin's outermost layer of the epidermis, the stratum corneum. It confers the skin's barrier function due to its structure that is analogous to a “brick and mortar”. The “bricks” are composed of flattened corneocytes enriched with proteins, glycoproteins, fatty acids, and cholesterol. The intercellular space, that comprises the “mortars”, is rich in bilayers composed of ceramides, cholesterol, fatty acids, and exhibits a polarity similar to that of butanol. In the past four decades three generations of TDDS have been developed. First-generation systems utilize diffusion of low molecular weight, lipophilic compounds. Second- and third-generation systems recognize that permeability of the stratum corneum is key. These strategies ablate/bypass the stratum corneum or utilize chemical enhancers, biochemical enhancers, and electromotive forces to increase permeability of the stratum corneum. Amongst different enhancement strategies, liposomes have been shown to disrupt the highly ordered structure of the stratum corneum and subsequently increase the skin's permeability.

In one embodiment herein, we describe the development of nanoporous particle-supported lipid bilayers (“protocells”) to serve as a TDDS. Protocells are formed by electrostatically fusing a liposome to a nanoporous silica-particle core. They synergistically combine the advantages of both inorganic nanoparticles and liposomes, such as tunable porosity, high surface area that is amenable to high capacity loading of disparate types of cargo, and a supported lipid bilayer (SLB) with tunable fluidity that can be modified with various molecules. These biophysical and biochemical properties allow the protocell to be modified for different applications. In our preliminary studies, using inductively coupled plasma mass spectroscopy, we have shown that 0.1-0.5 wt % of our standard protocell formulation (55% DOPE, 30% Cholesterol, 15% PEG-2000) dosed at 8.125 mg was able to cross full-thickness patient-derived abdominal skins. Additionally, we demonstrated that 0.3-2.4 wt % of protocells were able to cross partial thickness skin from which the stratum corneum was removed.

The nanoporous silica-particle core of the transdermal protocells has a high surface area, readily variable porosity, and surface chemistry that is easily modified. These properties make the protocell-core amenable to high-capacity loading of many different types of cargo. The protocell's supported lipid bilayer (SLB) has an inherently low immunogenicity. Additionally, the SLB provides a fluid surface to which peptides, polymers and other molecules can be conjugated in order to facilitate targeted cellular uptake. These biophysical and biochemical properties allow for the protocell to be optimized for a specific environment, facilitate penetration into the stratum corneum, and subsequently deliver disparate types of cargo via the transdermal route. Methods of treating a cancer are one example of a therapeutic use of the transdermal protocells of the invention. Related pharmaceutical compositions are also described.

In one embodiment, the invention provides a protocell comprising a plurality of negatively-charged, nanoporous, nanoparticulate silica cores that are modified with an amine-containing silane such as N-(2-aminoethyl)-3-aminopropyltrimethoxysilane (AEPTMS) and that (a) are loaded with a siRNA or ricin toxin A-chain and (b) that are encapsulated by and that support a lipid bilayer comprising one of more lipids selected from the group consisting of 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-dioleoyl-sn-glycero-3-[phosphor-L-serine] (DOPS), 1,2-dioleoyl-3-trimethylammonium-propane (18:1 DOTAP), 1,2-dioleoyl-sn-glycero-3-phospho-(1′-rac-glycerol) (DOPG), 1,2-dioleoyl-sn-glycero-3-phosphoethanolamine (DOPE), 1,2-dipalmitoyl-sn-glycero-3-phosphoethanolamine (DPPE), 1,2-dioleoyl-sn-glycero-3-phosphoethanolamine-N-[methoxy(polyethylene glycol)-2000] (18:1 PEG-2000 PE), 1,2-dipalmitoyl-sn-glycero-3-phosphoethanolamine-N-[methoxy(polyethylene glycol)-2000] (16:0 PEG-2000 PE), 1-Oleoyl-2-[12-[(7-nitro-2-1,3-benzoxadiazol-4-yl)amino]lauroyl]-sn-Glycero-3-Phosphocholine (18:1-12:0 NBD PC), 1-palmitoyl-2-{12-[(7-nitro-2-1,3-benzoxadiazol-4-yl)amino]lauroyl}-sn-glycero-3-phosphocholine (16:0-12:0 NBD PC), cholesterol and mixtures/combinations thereof, and wherein the lipid bilayer comprises a cationic lipid and one or more zwitterionic phospholipids wherein the lipid bilayer comprises CD47 molecules, either conjugated to the lipids or fused with a cell membrane lipid.

In the embodiment of the preceding paragraph, the lipid is preferably selected from the group consisting of 1,2-dioleoyl-3-trimethylammonium-propane (18:1 DOTAP) or 1,2-dioleoyl-sn-glycero-3-phospho-(1′-rac-glycerol) (DOPG), 1,2-dioleoyl-sn-glycero-3-phosphoethanolamine (DOPE) and mixtures thereof, and the protocell has at least one of the following characteristics: a BET surface area of greater than about 600 m²/g, a pore volume fraction of between about 60% to about 70%, a multimodal pore morphology composed of pores having an average diameter of between about 20 nm to about 30 nm, surface-accessible pores interconnected by pores having an average diameter of between about 5 nm to about 15 nm. Preferably, the protocell encapsulates around 10 nM of siRNA per 10¹⁰ nanoparticulate silica cores.

In still another embodiment, the invention provides a protocell comprising a plurality of negatively-charged, nanoporous, nanoparticulate silica cores that are modified with an amine-containing silane such as AEPTMS and that:
(a) are loaded with one or more siRNAs that target members of the cyclin superfamily selected from the group consisting of cyclin A2, cyclin B1, cyclin D1, and cyclin E; and
(b) that are encapsulated by and that support a lipid bilayer comprising one of more lipids selected from the group consisting of 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-dioleoyl-sn-glycero-3-[phosphor-L-serine] (DOPS), 1,2-dioleoyl-3-trimethylammonium-propane (18:1 DOTAP), 1,2-dioleoyl-sn-glycero-3-phospho-(1′-rac-glycerol) (DOPG), 1,2-dioleoyl-sn-glycero-3-phosphoethanolamine (DOPE), 1,2-dipalmitoyl-sn-glycero-3-phosphoethanolamine (DPPE), 1,2-dioleoyl-sn-glycero-3-phosphoethanolamine-N-[methoxy(polyethylene glycol)-2000] (18:1 PEG-2000 PE), 1,2-dipalmitoyl-sn-glycero-3-phosphoethanolamine-N-[methoxy(polyethylene glycol)-2000] (16:0 PEG-2000 PE), 1-Oleoyl-2-[12-[(7-nitro-2-1,3-benzoxadiazol-4-yl)amino]lauroyl]-sn-Glycero-3-Phosphocholine (18:1-12:0 NBD PC), 1-palmitoyl-2-{12-[(7-nitro-2-1,3-benzoxadiazol-4-yl)amino]lauroyl}-sn-glycero-3-phosphocholine (16:0-12:0 NBD PC), cholesterol and mixtures/combinations thereof, and wherein (1) the lipid bilayer is loaded with SP94 and an endosomolytic peptide, and (2) the protocell selectively binds to a hepatocellular carcinoma cell.

In the embodiment of the preceding paragraph, the lipid bilayer preferably comprises DOPC/DOPE/cholesterol/PEG-2000 in an approximately 55:5:30:10 mass ratio.

Methods of treating a cancer such as liver cancer are one example of a therapeutic use of the AEPTMS-modified protocells of the invention. Related pharmaceutical compositions are also described.

In another embodiment, the invention provides a protocell comprising a plurality of mesoporous, nanoparticulate silica cores that (a) are loaded with a siRNA that induces sequence-specific degradation of Nipah virus (NiV) nucleocapsid protein (NiV-N) mRNA and (b) that are encapsulated by and that support a lipid bilayer comprising one of more lipids selected from the group consisting of 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-dioleoyl-sn-glycero-3-[phosphor-L-serine] (DOPS), 1,2-dioleoyl-3-trimethylammonium-propane (18:1 DOTAP), 1,2-dioleoyl-sn-glycero-3-phospho-(1′-rac-glycerol) (DOPG), 1,2-dioleoyl-sn-glycero-3-phosphoethanolamine (DOPE), 1,2-dipalmitoyl-sn-glycero-3-phosphoethanolamine (DPPE), 1,2-dioleoyl-sn-glycero-3-phosphoethanolamine-N-[methoxy(polyethylene glycol)-2000] (18:1 PEG-2000 PE), 1,2-dipalmitoyl-sn-glycero-3-phosphoethanolamine-N-[methoxy(polyethylene glycol)-2000] (16:0 PEG-2000 PE), 1-Oleoyl-2-[12-[(7-nitro-2-1,3-benzoxadiazol-4-yl)amino]lauroyl]-sn-Glycero-3-Phosphocholine (18:1-12:0 NBD PC), 1-palmitoyl-2-{12-[(7-nitro-2-1,3-benzoxadiazol-4-yl)amino]lauroyl}-sn-glycero-3-phosphocholine (16:0-12:0 NBD PC), cholesterol and mixtures/combinations thereof.

In certain embodiments of the protocells of the preceding paragraph, the lipid bilayer comprises 1,2-dioleoyl-sn-glycero-3-phosphocholine (DOPC), 1,2-dioleoyl-sn-glycero-3-phosphoethanolamine (DOPE) a polyethylene glycol (PEG), a targeting peptide, and R8, and the mesoporous, nanoparticulate silica cores each have an average diameter of around 100 nm, an average surface area of greater than 1,000 m²/g and surface-accessible pores having an average diameter of between about 20 nm to about 25 nm, and have a siRNA load of around 1 μM per 10¹⁰ particles or greater. The targeting peptide preferably is a peptide that binds to ephrin B2 (EB2), and most preferably is TGAILHP (SEQ ID NO:18). Most preferably, the protocell comprises around 0.01 to around 0.02 wt % of TGAILHP, around 10 wt % PEG-2000 and around 0.500 wt % of R8.

Methods of treating a subject who is infected by, or at risk of infection with Nipah virus (NiV) are one example of a therapeutic use of protocells of the invention comprising a siRNA that induces sequence-specific degradation of Nipah virus (NiV) nucleocapsid protein (NiV-N) mRNA. Related pharmaceutical compositions are also described.

Other aspects of embodiments of the present invention are directed to pharmaceutical compositions. Pharmaceutical compositions according to the present invention comprise a population of protocells which may be the same or different and are formulated in combination with a pharmaceutically acceptable carrier, additive or excipient. The protocells may be formulated alone or in combination with another bioactive agent (such as an additional anti-cancer agent or an antiviral agent) depending upon the disease treated and the route of administration (as otherwise described herein). These compositions comprise protocells as modified for a particular purpose (e.g. therapy, including cancer therapy, or diagnostics, including the monitoring of cancer therapy). Pharmaceutical compositions comprise an effective population of protocells for a particular purpose and route of administration in combination with a pharmaceutically acceptable carrier, additive or excipient.

An embodiment of the present invention also relates to methods of utilizing the novel protocells as described herein. Thus, in alternative embodiments, the present invention relates to a method of treating a disease and/or condition comprising administering to a patient or subject in need an effective amount of a pharmaceutical composition as otherwise described herein. The pharmaceutical compositions according to the present invention are particularly useful for the treatment of a number disease states, especially including cancer, and disease states or conditions which occur secondary to cancer or are the cause of cancer (in particular, HBV and/or HCV infections).

In further alternative aspects, the present invention relates to methods of diagnosing cancer, the method comprising administering a pharmaceutical composition comprising a population of protocells which have been modified to deliver a diagnostic agent or reporter imaging agent selectively to cancer cells to identify cancer in the patient. In this method, protocells according to the present invention may be adapted to target cancer cells through the inclusion of at least one targeting peptide which binds to cancer cells which express polypeptides or more generally, surface receptors or cell membrane components, which are the object of the targeting peptide and through the inclusion of a reporter component (including an imaging agent) of the protocell targeted to the cancer cell, may be used to identify the existence and size of cancerous tissue in a patient or subject by comparing a signal from the reporter with a standard. The standard may be obtained for example, from a population of healthy patients or patients known to have a disease for which diagnosis is made. Once diagnosed, appropriate therapy with pharmaceutical compositions according to the present invention, or alternative therapy may be implemented.

In still other aspects of the invention, the compositions according to the present invention may be used to monitor the progress of therapy of a particular disease state and/or condition, including therapy with compositions according to the present invention. In this aspect of the invention, a composition comprising a population of protocells which are specific for cancer cell binding and include a reporter component may be administered to a patient or subject undergoing therapy such that progression of the therapy of the disease state can be monitored.

Pharmaceutical compositions according to the present invention comprise an effective population of protocells as otherwise described herein formulated to effect an intended result (e.g. therapeutic result and/or diagnostic analysis, including the monitoring of therapy) formulated in combination with a pharmaceutically acceptable carrier, additive or excipient. The protocells within the population of the composition may be the same or different depending upon the desired result to be obtained. Pharmaceutical compositions according to the present invention may also comprise an addition bioactive agent or drug, such as an anticancer agent or an antiviral agent, for example, an anti-HIV, anti-HBV or an anti-HCV agent.

Generally, dosages and routes of administration of the compound are determined according to the size and condition of the subject, according to standard pharmaceutical practices. Dose levels employed can vary widely, and can readily be determined by those of skill in the art. Typically, amounts in the milligram up to gram quantities are employed. The composition may be administered to a subject by various routes, e.g. orally, transdermally, perineurally or parenterally, that is, by intravenous, subcutaneous, intraperitoneal, intrathecal or intramuscular injection, among others, including buccal, rectal and transdermal administration. Subjects contemplated for treatment according to the method of the invention include humans, companion animals, laboratory animals, and the like. The invention contemplates immediate and/or sustained/controlled release compositions, including compositions which comprise both immediate and sustained release formulations. This is particularly true when different populations of protocells are used in the pharmaceutical compositions or when additional bioactive agent(s) are used in combination with one or more populations of protocells as otherwise described herein.

Formulations containing the compounds according to the present invention may take the form of liquid, solid, semi-solid or lyophilized powder forms, such as, for example, solutions, suspensions, emulsions, sustained-release formulations, tablets, capsules, powders, suppositories, creams, ointments, lotions, aerosols, patches or the like, preferably in unit dosage forms suitable for simple administration of precise dosages.

Pharmaceutical compositions according to the present invention typically include a conventional pharmaceutical carrier or excipient and may additionally include other medicinal agents, carriers, adjuvants, additives and the like. Preferably, the composition is about 0.1% to about 85%, about 0.5% to about 75% by weight of a compound or compounds of the invention, with the remainder consisting essentially of suitable pharmaceutical excipients.

An injectable composition for parenteral administration (e.g. intravenous, intramuscular or intrathecal) will typically contain the compound in a suitable i.v. solution, such as sterile physiological salt solution. The composition may also be formulated as a suspension in an aqueous emulsion.

Liquid compositions can be prepared by dissolving or dispersing the population of protocells (about 0.5% to about 20% by weight or more), and optional pharmaceutical adjuvants, in a carrier, such as, for example, aqueous saline, aqueous dextrose, glycerol, or ethanol, to form a solution or suspension. For use in an oral liquid preparation, the composition may be prepared as a solution, suspension, emulsion, or syrup, being supplied either in liquid form or a dried form suitable for hydration in water or normal saline.

For oral administration, such excipients include pharmaceutical grades of mannitol, lactose, starch, magnesium stearate, sodium saccharine, talcum, cellulose, glucose, gelatin, sucrose, magnesium carbonate, and the like. If desired, the composition may also contain minor amounts of non-toxic auxiliary substances such as wetting agents, emulsifying agents, or buffers.

When the composition is employed in the form of solid preparations for oral administration, the preparations may be tablets, granules, powders, capsules or the like. In a tablet formulation, the composition is typically formulated with additives, e.g. an excipient such as a saccharide or cellulose preparation, a binder such as starch paste or methyl cellulose, a filler, a disintegrator, and other additives typically used in the manufacture of medical preparations.

Methods for preparing such dosage forms are known or is apparent to those skilled in the art; for example, see Remington's Pharmaceutical Sciences (17th Ed., Mack Pub. Co., 1985). The composition to be administered will contain a quantity of the selected compound in a pharmaceutically effective amount for therapeutic use in a biological system, including a patient or subject according to the present invention.

Methods of treating patients or subjects in need for a particular disease state or infection (especially including cancer and/or a HBV, HCV or HIV infection) comprise administration an effective amount of a pharmaceutical composition comprising therapeutic protocells and optionally at least one additional bioactive (e.g. antiviral) agent according to the present invention.

Diagnostic methods according to the present invention comprise administering to a patient in need (a patient suspected of having cancer) an effective amount of a population of diagnostic protocells (e.g., protocells which comprise a target species, such as a targeting peptide which binds selectively to cancer cells and a reporter component to indicate the binding of the protocells to cancer cells if the cancer cells are present) whereupon the binding of protocells to cancer cells as evidenced by the reporter component (moiety) will enable a diagnosis of the existence of cancer in the patient.

An alternative of the diagnostic method of the present invention can be used to monitor the therapy of cancer or other disease state in a patient, the method comprising administering an effective population of diagnostic protocells (e.g., protocells which comprise a target species, such as a targeting peptide which binds selectively to cancer cells or other target cells and a reporter component to indicate the binding of the protocells to cancer cells if the cancer cells are present) to a patient or subject prior to treatment, determining the level of binding of diagnostic protocells to target cells in said patient and during and/or after therapy, determing the level of binding of diagnostic protocells to target cells in said patient, whereupon the difference in binding before the start of therapy in the patient and during and/or after therapy will evidence the effectiveness of therapy in the patient, including whether the patient has completed therapy or whether the disease state has been inhibited or eliminated (including remission of a cancer).

### VARIOUS EMBODIMENTS

### Embodiment 1

A porous protocell comprising a nanoporous silica or metal oxide core with a supported lipid bilayer comprising at least one CD47 molecule or active fragment thereof.

### Embodiment 2

A cell-targeting porous protocell comprising:

a nanoporous silica or metal oxide core with a supported lipid bilayer comprising at least one CD47 molecule or active fragment thereof and at least one further component selected from the group consisting of
a cell targeting species;
a fusogenic peptide that promotes endosomal escape of protocells and encapsulated DNA,
other cargo comprising at least one cargo component selected from the group consisting of double stranded linear DNA or plasmid DNA;
a drug;
an imaging agent,
RNA, including small interfering RNA, small hairpin RNA, microRNA, antisense RNA or a mixture thereof,
wherein one of said cargo components is optionally conjugated further with a nuclear localization sequence.

### Embodiment 3

The protocell according to embodiment 1, wherein said silica core is spherical and ranges in diameter from about 10 nm to about 250 nm.

### Embodiment 4

The protocell according to embodiment 1 or 2 wherein said silica core has a mean diameter of about 30 to about 100 nm.

### Embodiment 5

The protocell according to either of embodiments 2 or 3 wherein said silica core is monodisperse or polydisperse in size distribution.

### Embodiment 6

The protocell according to either of embodiments 2 or 3 wherein said silica core is monodisperse.

### Embodiment 7

The protocell according to either of embodiments 2 or 3 wherein said silica core is polydisperse.

### Embodiment 8

The protocell according to any of embodiments 1-7 wherein said lipid bilayer is comprised of lipids selected from the group consisting of 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-dioleoyl-sn-glycero-3-[phosphor-L-serine] (DOPS), 1,2-dioleoyl-3-trimethylammonium-propane (18:1 DOTAP), 1,2-dioleoyl-sn-glycero-3-phospho-(1′-rac-glycerol) (DOPG), 1,2-dioleoyl-sn-glycero-3-phosphoethanolamine (DOPE), 1,2-dipalmitoyl-sn-glycero-3-phosphoethanolamine (DPPE), 1,2-dioleoyl-sn-glycero-3-phosphoethanolamine-N-[methoxy(polyethylene glycol)-2000] (18:1 PEG-2000 PE), 1,2-dipalmitoyl-sn-glycero-3-phosphoethanolamine-N-[methoxy(polyethylene glycol)-2000] (16:0 PEG-2000 PE), 1-Oleoyl-2-[12-[(7-nitro-2-1,3-benzoxadiazol-4-yl)amino]lauroyl]-sn-Glycero-3-Phosphocholine (18:1-12:0 NBD PC), 1-palmitoyl-2-{12-[(7-nitro-2-1,3-benzoxadiazol-4-yl)amino]lauroyl}-sn-glycero-3-phosphocholine (16:0-12:0 NBD PC), cholesterol and mixtures thereof.

### Embodiment 9

The protocell according to any of embodiments 1-8 wherein said lipid bilayer comprises DOPC in combination with DOPE.

### Embodiment 10

The protocell according to any of embodiments 1-8 wherein said lipid bilayer comprises DOTAP, DOPG, DOPC or mixtures thereof.

### Embodiment 11

The protocell according to any of embodiments 1-8 wherein said lipid bilayer comprises DOPG and DOPC.

### Embodiment 12

The protocell according to any of embodiments 9-11 wherein said lipid bilayer further comprises cholesterol.

### Embodiment 13

The protocell according to any of embodiments 1-8 wherein said lipid bilayer comprises DOPC in combination with about 5 wt % DOPE, about 30 wt % cholesterol, and about 10 wt % PEG-2000 PE (18:1).

### Embodiment 14

The protocell according to any of embodiments 1-8 wherein lipid bilayer comprises about 5% by weight DOPE, about 5% by weight PEG, about 30% by weight cholesterol, about 60% by weight DOPC and/or DPPC.

### Embodiment 15

The protocell according to embodiment 14 wherein said PEG is conjugated to said DOPE.

### Embodiment 16

The protocell according to any of embodiments 2-15 wherein said targeting species is a targeting peptide.

### Embodiment 17

The protocell according to embodiment 16 wherein said targeting peptide is a SP94 peptide.

### Embodiment 18

The protocell according to embodiment 17 wherein said targeting peptide is SEQ ID NO: 6, SEQ ID NO: 7 or SEQ ID NO: 8.

### Embodiment 19

The protocell according to embodiment 16 wherein said targeting peptide is a MET binding peptide according to SEQ ID NO: 1, SEQ ID NO: 2, SEQ I.D. NO: 3, SEQ I.D. No. 4 or SEQ ID NO: 5.

### Embodiment 20

The protocell according to any of embodiments 2-19 wherein said fusogenic protein is H5WYG peptide (SEQ ID NO: 13) or an eight mer of polyarginine (SEQ ID NO: 14).

### Embodiment 21

The protocell according to embodiment 20 wherein said fusogenic peptide is SEQ ID NO: 13.

### Embodiment 22

The protocell according to any of embodiments 1-21 comprising plasmid DNA, wherein said plasmid DNA is optionally modified to express a nuclear localization sequence.

### Embodiment 23

The protocell according to 22 wherein said plasmid DNA is supercoiled or packaged plasmid DNA

### Embodiment 24

The protocell according to embodiment 23 wherein said DNA is both supercoiled and packaged plasmid DNA.

### Embodiment 25

The protocell according to any of embodiments 22-24 wherein said plasmid DNA is modified to express a nuclear localization sequence.

### Embodiment 26

The protocell according to any of embodiments 22-25 wherein said DNA is histone-packaged supercoiled plasmid DNA comprises a mixture of human histone proteins.

### Embodiment 27

The protocell according to embodiment 26 wherein said mixture of histones consists of H1, H2A, H2B, H3, and H4.

### Embodiment 28

The protocell according to embodiment 26 wherein said mixture of histones is H1, H2A, H2B, H3 and H4 is in a weight ratio of 1:2:2:2:2.

### Embodiment 29

The protocell according to any of embodiments 2-28 wherein said plasmid DNA is capable of expressing a polypeptide toxin, a small hairpin RNA (shRNA) or a small interfering RNA (siRNA).

### Embodiment 30

The protocell according to embodiment 29 wherein said polypeptide toxin is selected from the group consisting of ricin toxin chain-A or diphtheria toxin chain-A.

### Embodiment 31

The protocell according to embodiment 2 or 29 wherein said shRNA or said siRNA induces apoptosis of a cell.

### Embodiment 32

The protocell according to any of embodiments 2-31 wherein said DNA is capable of expressing a reporter protein.

### Embodiment 33

The protocell according to embodiment 32 wherein said reporter protein is green fluorescent protein or red fluorescent protein.

### Embodiment 34

The protocell according to any of embodiments 2-33 wherein said nuclear localization sequence is a peptide according to SEQ ID NO: 9, SEQ ID NO: 10, SEQ ID NO: 11 or SEQ ID NO: 12.

### Embodiment 35

The protocell according to any of embodiments 2-34 wherein said nuclear localization sequence is a peptide according to SEQ ID NO: 9.

### Embodiment 36

The protocell according to any of embodiments 2-35 further comprising as a drug an anticancer agent.

### Embodiment 37

The protocell according to embodiment 36 wherein said anticancer agent is everolimus, trabectedin, abraxane, TLK 286, AV-299, DN-101, pazopanib, GSK690693, RTA 744, ON 0910.Na, AZD 6244 (ARRY-142886), AMN-107, TKI-258, GSK461364, AZD 1152, enzastaurin, vandetanib, ARQ-197, MK-0457, MLN8054, PHA-739358, R-763, AT-9263, a FLT-3 inhibitor, a VEGFR inhibitor, an EGFR TK inhibitor, an aurora kinase inhibitor, a PIK-1 modulator, a Bcl-2 inhibitor, an HDAC inhbitor, a c-MET inhibitor, a PARP inhibitor, a Cdk inhibitor, an EGFR TK inhibitor, an IGFR-TK inhibitor, an anti-HGF antibody, a PI3 kinase inhibitors, an AKT inhibitor, a JAK/STAT inhibitor, a checkpoint-1 or 2 inhibitor, a focal adhesion kinase inhibitor, a Map kinase kinase (mek) inhibitor, a VEGF trap antibody, pemetrexed, erlotinib, dasatanib, nilotinib, decatanib, panitumumab, amrubicin, oregovomab, Lep-etu, nolatrexed, azd2171, batabulin, ofatumumab, zanolimumab, edotecarin, tetrandrine, rubitecan, tesmilifene, oblimersen, ticilimumab, ipilimumab, gossypol, Bio 111, 131-I-TM-601, ALT-110, BIO 140, CC 8490, cilengitide, gimatecan, IL13-PE38QQR, INO 1001, IPdR₁ KRX-0402, lucanthone, LY 317615, neuradiab, vitespan, Rta 744, Sdx 102, talampanel, atrasentan, Xr 311, romidepsin, ADS-100380, sunitinib, 5-fluorouracil, vorinostat, etoposide, gemcitabine, doxorubicin, 5′-deoxy-5-fluorouridine, vincristine, temozolomide, ZK-304709, seliciclib; PD0325901, AZD-6244, capecitabine, L-Glutamic acid, N-[4-[2-(2-amino-4,7-dihydro-4-oxo-1H-pyrrolo[2,3-d]pyrimidin-5-yl)ethyl]benzoyl]-, disodium salt, heptahydrate, camptothecin, PEG-labeled irinotecan, tamoxifen, toremifene citrate, anastrazole, exemestane, letrozole, DES(diethylstilbestrol), estradiol, estrogen, conjugated estrogen, bevacizumab, IMC-1C11, CHIR-258,); 3-[5-(methylsulfonylpiperadinemethyl)-indolylj-quinolone, vatalanib, AG-013736, AVE-0005, the acetate salt of [D-Ser(Bu t) 6,Azgly 10] (pyro-Glu-His-Trp-Ser-Tyr-D-Ser(Bu t)-Leu-Arg-Pro-Azgly-NH₂ acetate [C₅₉H₈₄N₁₈Oi₄-(C₂H₄O₂)_{X} where x=1 to 2.4], goserelin acetate, leuprolide acetate, triptorelin pamoate, medroxyprogesterone acetate, hydroxyprogesterone caproate, megestrol acetate, raloxifene, bicalutamide, flutamide, nilutamide, megestrol acetate, CP-724714; TAK-165, HKI-272, erlotinib, lapatanib, canertinib, ABX-EGF antibody, erbitux, EKB-569, PKI-166, GW-572016, Ionafamib, BMS-214662, tipifarnib; amifostine, NVP-LAQ824, suberoyl analide hydroxamic acid, valproic acid, trichostatin A, FK-228, SU11248, sorafenib, KRN951, aminoglutethimide, amsacrine, anagrelide, L-asparaginase, Bacillus Calmette-Guerin (BCG) vaccine, bleomycin, buserelin, busulfan, carboplatin, carmustine, chlorambucil, cisplatin, cladribine, clodronate, cyproterone, cytarabine, dacarbazine, dactinomycin, daunorubicin, diethylstilbestrol, epirubicin, fludarabine, fludrocortisone, fluoxymesterone, flutamide, gemcitabine, gleevac, hydroxyurea, idarubicin, ifosfamide, imatinib, leuprolide, levamisole, lomustine, mechlorethamine, melphalan, 6-mercaptopurine, mesna, methotrexate, mitomycin, mitotane, mitoxantrone, nilutamide, octreotide, oxaliplatin, pamidronate, pentostatin, plicamycin, porfimer, procarbazine, raltitrexed, rituximab, streptozocin, teniposide, testosterone, thalidomide, thioguanine, thiotepa, tretinoin, vindesine, 13-cis-retinoic acid, phenylalanine mustard, uracil mustard, estramustine, altretamine, floxuridine, 5-deooxyuridine, cytosine arabinoside, 6-mecaptopurine, deoxycoformycin, calcitriol, valrubicin, mithramycin, vinblastine, vinorelbine, topotecan, razoxin, marimastat, COL-3, neovastat, BMS-275291, squalamine, endostatin, SU5416, SU6668, EMD121974, interleukin-12, IM862, angiostatin, vitaxin, droloxifene, idoxyfene, spironolactone, finasteride, cimitidine, trastuzumab, denileukin diftitox, gefitinib, bortezimib, paclitaxel, cremophor-free paclitaxel, docetaxel, epithilone B, BMS-247550, BMS-310705, droloxifene, 4-hydroxytamoxifen, pipendoxifene, ERA-923, arzoxifene, fulvestrant, acolbifene, lasofoxifene, idoxifene, TSE-424, HMR-3339, ZK186619, topotecan, PTK787/ZK 222584, VX-745, PD 184352, rapamycin, 40-O-(2-hydroxyethyl)-rapamycin, temsirolimus, AP-23573, RAD001, ABT-578, BC-210, LY294002, LY292223, LY292696, LY293684, LY293646, wortmannin, ZM336372, L-779,450, PEG-filgrastim, darbepoetin, erythropoietin, granulocyte colony-stimulating factor, zolendronate, prednisone, cetuximab, granulocyte macrophage colony-stimulating factor, histrelin, pegylated interferon alfa-2a, interferon alfa-2a, pegylated interferon alfa-2b, interferon alfa-2b, azacitidine, PEG-L-asparaginase, lenalidomide, gemtuzumab, hydrocortisone, interleukin-11, dexrazoxane, alemtuzumab, all-transretinoic acid, ketoconazole, interleukin-2, megestrol, immune globulin, nitrogen mustard, methylprednisolone, ibritgumomab tiuxetan, androgens, decitabine, hexamethylmelamine, bexarotene, tositumomab, arsenic trioxide, cortisone, editronate, mitotane, cyclosporine, liposomal daunorubicin, Edwina-asparaginase, strontium 89, casopitant, netupitant, an NK-1 receptor antagonists, palonosetron, aprepitant, diphenhydramine, hydroxyzine, metoclopramide, lorazepam, alprazolam, haloperidol, droperidol, dronabinol, dexamethasone, methylprednisolone, prochlorperazine, granisetron, ondansetron, dolasetron, tropisetron, pegfilgrastim, erythropoietin, epoetin alfa, darbepoetin alfa or a mixture thereof.

### Embodiment 38

The protocell according to any of embodiments 2-37 wherein said drug comprises an antiviral agent.

### Embodiment 39

The protocell according to embodiment 38 wherein said antiviral agent is an anti-HIV agent, an anti-HBV agent or an anti-HCV agent.

### Embodiment 40

The protocell according to any of embodiments 1-39 wherein said CD47 molecule(s) is conjugated to a lipid in said lipid bilayer.

### Embodiment 41

The protocell according to any of embodiments 1-39 wherein said lipid bilayer comprises a cellular plasma membrane containing CD47 molecules.

### Embodiment 42

A protocell comprising a nanoporous silica core with a supported lipid bilayer and a MET binding peptide according to SEQ ID NO: 1, SEQ ID NO: 2, SEQ ID NO:3, SEQ ID. NO: 4 or SEQ ID NO: 5, wherein said lipid bilayer comprises at least one CD47 molecule or an active fragment thereof.

### Embodiment 43

The protocell according to embodiment 42 wherein said MET binding peptide is a peptide according to SEQ ID NO:1.

### Embodiment 44

The protocell according to embodiment 42 or 43 wherein said MET binding peptide is conjugated to said lipid bilayer.

### Embodiment 45

The protocell according to any of embodiments 42-44 wherein said protocell further comprises at least one component selected from the group consisting of a fusogenic peptide that promotes endosomal escape of protocells and encapsulated DNA; a plasmid DNA; double stranded linear DNA, a drug; an imaging agent, small interfering RNA, small hairpin RNA and micro RNA wherein said plasmid DNA, said drug, said imaging agent and/or said RNA are further conjugated with a nuclear localization sequence.

### Embodiment 46

A protocell according to embodiment 45 wherein said drug comprises at least one anticancer agent.

### Embodiment 47

The protocell according to embodiment 46 wherein said anticancer agent is selected from the group consisting of everolimus, trabectedin, abraxane, TLK 286, AV-299, DN-101, pazopanib, GSK690693, RTA 744, ON 0910.Na, AZD 6244 (ARRY-142886), AMN-107, TKI-258, GSK461364, AZD 1152, enzastaurin, vandetanib, ARQ-197, MK-0457, MLN8054, PHA-739358, R-763, AT-9263, a FLT-3 inhibitor, a VEGFR inhibitor, an EGFR TK inhibitor, an aurora kinase inhibitor, a PIK-1 modulator, a Bcl-2 inhibitor, an HDAC inhbitor, a c-MET inhibitor, a PARP inhibitor, a Cdk inhibitor, an EGFR TK inhibitor, an IGFR-TK inhibitor, an anti-HGF antibody, a PI3 kinase inhibitors, an AKT inhibitor, a JAK/STAT inhibitor, a checkpoint-1 or 2 inhibitor, a focal adhesion kinase inhibitor, a Map kinase kinase (mek) inhibitor, a VEGF trap antibody, pemetrexed, erlotinib, dasatanib, nilotinib, decatanib, panitumumab, amrubicin, oregovomab, Lep-etu, nolatrexed, azd2171, batabulin, ofatumumab, zanolimumab, edotecarin, tetrandrine, rubitecan, tesmilifene, oblimersen, ticilimumab, ipilimumab, gossypol, Bio 111, 131-I-TM-601, ALT-110, BIO 140, CC 8490, cilengitide, gimatecan, IL13-PE38QQR, INO 1001, IPdR₁ KRX-0402, lucanthone, LY 317615, neuradiab, vitespan, Rta 744, Sdx 102, talampanel, atrasentan, Xr 311, romidepsin, ADS-100380, sunitinib, 5-fluorouracil, vorinostat, etoposide, gemcitabine, doxorubicin, 5′-deoxy-5-fluorouridine, vincristine, temozolomide, ZK-304709, seliciclib; PD0325901, AZD-6244, capecitabine, L-Glutamic acid, N-[4-[2-(2-amino-4,7-dihydro-4-oxo-1H-pyrrolo[2,3-d]pyrimidin-5-yl)ethyl]benzoyl]-, disodium salt, heptahydrate, camptothecin, PEG-labeled irinotecan, tamoxifen, toremifene citrate, anastrazole, exemestane, letrozole, DES(diethylstilbestrol), estradiol, estrogen, conjugated estrogen, bevacizumab, IMC-1C11, CHIR-258,); 3-[5-(methylsulfonylpiperadinemethyl)-indolylj-quinolone, vatalanib, AG-013736, AVE-0005, the acetate salt of [D-Ser(Bu t) 6,Azgly 10] (pyro-Glu-His-Trp-Ser-Tyr-D-Ser(Bu t)-Leu-Arg-Pro-Azgly-NH₂ acetate [C₅₉H₈₄N₁₈Oi₄-(C₂H₄O₂)_{X} where x=1 to 2.4], goserelin acetate, leuprolide acetate, triptorelin pamoate, medroxyprogesterone acetate, hydroxyprogesterone caproate, megestrol acetate, raloxifene, bicalutamide, flutamide, nilutamide, megestrol acetate, CP-724714; TAK-165, HKI-272, erlotinib, lapatanib, canertinib, ABX-EGF antibody, erbitux, EKB-569, PKI-166, GW-572016, Ionafarnib, BMS-214662, tipifarnib; amifostine, NVP-LAQ824, suberoyl analide hydroxamic acid, valproic acid, trichostatin A, FK-228, SU11248, sorafenib, KRN951, aminoglutethimide, amsacrine, anagrelide, L-asparaginase, Bacillus Calmette-Guerin (BCG) vaccine, bleomycin, buserelin, busulfan, carboplatin, carmustine, chlorambucil, cisplatin, cladribine, clodronate, cyproterone, cytarabine, dacarbazine, dactinomycin, daunorubicin, diethylstilbestrol, epirubicin, fludarabine, fludrocortisone, fluoxymesterone, flutamide, gemcitabine, gleevac, hydroxyurea, idarubicin, ifosfamide, imatinib, leuprolide, levamisole, lomustine, mechlorethamine, melphalan, 6-mercaptopurine, mesna, methotrexate, mitomycin, mitotane, mitoxantrone, nilutamide, octreotide, oxaliplatin, pamidronate, pentostatin, plicamycin, porfimer, procarbazine, raltitrexed, rituximab, streptozocin, teniposide, testosterone, thalidomide, thioguanine, thiotepa, tretinoin, vindesine, 13-cis-retinoic acid, phenylalanine mustard, uracil mustard, estramustine, altretamine, floxuridine, 5-deooxyuridine, cytosine arabinoside, 6-mecaptopurine, deoxycoformycin, calcitriol, valrubicin, mithramycin, vinblastine, vinorelbine, topotecan, razoxin, marimastat, COL-3, neovastat, BMS-275291, squalamine, endostatin, SU5416, SU6668, EMD121974, interleukin-12, IM862, angiostatin, vitaxin, droloxifene, idoxyfene, spironolactone, finasteride, cimitidine, trastuzumab, denileukin diftitox, gefitinib, bortezimib, paclitaxel, cremophor-free paclitaxel, docetaxel, epithilone B, BMS-247550, BMS-310705, droloxifene, 4-hydroxytamoxifen, pipendoxifene, ERA-923, arzoxifene, fulvestrant, acolbifene, lasofoxifene, idoxifene, TSE-424, HMR-3339, ZK186619, topotecan, PTK787/ZK 222584, VX-745, PD 184352, rapamycin, 40-O-(2-hydroxyethyl)-rapamycin, temsirolimus, AP-23573, RAD001, ABT-578, BC-210, LY294002, LY292223, LY292696, LY293684, LY293646, wortmannin, ZM336372, L-779,450, PEG-filgrastim, darbepoetin, erythropoietin, granulocyte colony-stimulating factor, zolendronate, prednisone, cetuximab, granulocyte macrophage colony-stimulating factor, histrelin, pegylated interferon alfa-2a, interferon alfa-2a, pegylated interferon alfa-2b, interferon alfa-2b, azacitidine, PEG-L-asparaginase, lenalidomide, gemtuzumab, hydrocortisone, interleukin-11, dexrazoxane, alemtuzumab, all-transretinoic acid, ketoconazole, interleukin-2, megestrol, immune globulin, nitrogen mustard, methylprednisolone, ibritgumomab tiuxetan, androgens, decitabine, hexamethylmelamine, bexarotene, tositumomab, arsenic trioxide, cortisone, editronate, mitotane, cyclosporine, liposomal daunorubicin, Edwina-asparaginase, strontium 89, casopitant, netupitant, an NK-1 receptor antagonists, palonosetron, aprepitant, diphenhydramine, hydroxyzine, metoclopramide, lorazepam, alprazolam, haloperidol, droperidol, dronabinol, dexamethasone, methylprednisolone, prochlorperazine, granisetron, ondansetron, dolasetron, tropisetron, pegfilgrastim, erythropoietin, epoetin alfa, darbepoetin alfa and mixtures thereof.

### Embodiment 48

The protocell according to embodiment 45 wherein said drug comprises at least one antiviral agent.

### Embodiment 49

The protocell according to embodiment 48 wherein said antiviral agent is an anti-HIV agent, an anti-HBV agent, an anti-HCV agent or mixtures thereof.

### Embodiment 50

A protocell according to any of embodiments 45 wherein said DNA is capable of expressing at least one reporter molecule.

### Embodiment 51

The protocell according to any of embodiments 41-47 comprising plasmid DNA, wherein said plasmid DNA is optionally modified to express a nuclear localization sequence.

### Embodiment 52

The protocell according to 51 wherein said DNA is supercoiled or packaged plasmid DNA

### Embodiment 53

The protocell according to embodiment 51 wherein said DNA is both supercoiled and packaged plasmid DNA.

### Embodiment 54

The protocell according to any of embodiments 51-53 wherein said plasmid DNA is modified to express a nuclear localization sequence.

### Embodiment 55

The protocell according to any of embodiments 50-54 wherein said DNA is histone-packaged supercoiled plasmid DNA comprises a mixture of human histone proteins.

### Embodiment 56

The protocell according to embodiment 55 wherein said mixture of histones consists of H1, H2A, H2B, H3, and H4.

### Embodiment 57

The protocell according to embodiment 56 wherein said mixture of histones is H1, H2A, H2B, H3 and H4 is in a weight ratio of 1:2:2:2:2.

### Embodiment 58

The protocell according to any of embodiments 51-57 wherein said plasmid DNA is capable of expressing a polypeptide toxin, a small hairpin RNA (shRNA) or a small interfering RNA (siRNA).

### Embodiment 59

The protocell according to embodiment 58 wherein said polypeptide toxin is selected from the group consisting of ricin toxin chain-A or diphtheria toxin chain-A.

### Embodiment 60

The protocell according to embodiment 58 wherein said shRNA or said siRNA induces apoptosis of a cell.

### Embodiment 61

The protocell according to any of embodiments 51-60 wherein said plasmid DNA is capable of expressing a reporter protein.

### Embodiment 62

The protocell according to embodiment 61 wherein said reporter protein is green fluorescent protein or red fluorescent protein.

### Embodiment 63

The protocell according to any of embodiments 45-62 wherein said nuclear localization sequence is a peptide according to SEQ ID NO: 9, SEQ ID NO: 10, SEQ ID NO: 11 or SEQ ID NO: 12.

### Embodiment 64

The protocell according to embodiment 63 wherein said nuclear localization sequence is a peptide according to SEQ ID NO: 9.

### Embodiment 65

The protocell according to any of embodiments 42-64 wherein said CD47 molecule(s) is conjugated to a lipid in said lipid bilayer.

### Embodiment 66

The protocell according to any of embodiments 1-39 wherein said lipid bilayer comprises a cellular plasma membrane containing CD47 molecules.

### Embodiment 67

A pharmaceutical composition comprising a population of protocells according to any of embodiments 1-66 in an amount effective for effecting a therapeutic effect in combination with a pharmaceutically acceptable carrier, additive or excipient.

### Embodiment 68

The composition according to embodiment 67 further comprising a drug which is not disposed as cargo within the protocell.

### Embodiment 69

The composition according to embodiment 68 wherein said drug is an anti-cancer agent or an anti-viral agent.

### Embodiment 70

The composition according to embodiment 69 wherein said anti-viral agent is an anti-HIV agent, anti-HBV agent, an anti-HCV agent or mixtures thereof.

### Embodiment 71

The composition according to any of embodiments 67-70 in parenteral dosage form.

### Embodiment 72

The composition according to embodiment 71 wherein said dosage form is intradermal, intramuscular, intraosseous, intraperitoneal, intravenous, subcutaneous or intrathecal.

### Embodiment 73

The composition according to any of embodiments 67-70 in topical or transdermal dosage form.

### Embodiment 74

A MET binding peptide according to SEQ ID NO: 1, SEQ ID NO: 2, SEQ ID NO:3, SEQ ID. NO: 4 or SEQ ID NO: 5.

### Embodiment 75

A MET binding peptide of embodiment 74 according to SEQ ID NO: 1.

### Embodiment 76

A pharmaceutical composition comprising a MET binding peptide according to embodiment 74 or 75.

### Embodiment 77

A pharmaceutical composition comprising a population of protocells which comprise a targeting peptide so that the protocells selectively bind to hepatocellular cancer cells in combination with an anticancer agent and an anti-HBV agent an anti-HCV agent or a mixture thereof, wherein said protocells comprise at least one CD47 molecule or active fragment thereof, wherein said CD47 molecule or active fragment thereof is conjugated to a lipid of a lipid bilayer of said protocell or said CD47 molecule is incorporated into said lipid bilayer through the addition of a cellular plasma membrane fused into said lipid bilayer.

### Embodiment 78

The composition according to embodiment 77 wherein said targeting peptide is selected from the group consisting of a S94 peptide, a MET binding peptide or mixtures thereof.

### Embodiment 79

The composition according to embodiment 77 wherein said anticancer agent is nexavar (sorafenib), sunitinib, bevacizumab, tarceva (erlotinib), tykerb (lapatinib) or a mixture thereof.

### Embodiment 80

The composition according to any of embodiments 77-79 wherein said anti-HBV agent is Hepsera (adefovir dipivoxil), lamivudine, entecavir, telbivudine, tenofovir, emtricitabine, clevudine, valtoricitabine, amdoxovir, pradefovir, racivir, BAM 205, nitazoxanide, UT 231-B, Bay 41-4109, EHT899, zadaxin (thymosin alpha-1) or a mixture thereof.

### Embodiment 81

The composition according to any of embodiments 77-80 wherein said anti-HCV agent is boceprevir, daclatasvir, asunapavir, INX-189, FV-100, NM 283, VX-950 (telaprevir), SCH 50304, TMC435, VX-500, BX-813, SCH503034, R1626, ITMN-191 (R7227), R7128, PF-868554, TT033, CGH-759, GI 5005, MK-7009, SIRNA-034, MK-0608, A-837093, GS 9190, GS 9256, GS 9451, GS 5885, GS 6620, GS 9620, GS9669, ACH-1095, ACH-2928, GSK625433, TG4040 (MVA-HCV), A-831, F351, NS5A, NS4B, ANA598, A-689, GNI-104, IDX102, ADX184, ALS-2200, ALS-2158, BI 201335, BI 207127, BIT-225, BIT-8020, GL59728, GL60667, PSI-938, PSI-7977, PSI-7851, SCY-635, ribavirin, pegylated interferon, PHX1766, SP-30 or a mixture thereof.

### Embodiment 82

A method of treating cancer comprising administering to a patient in need an effective amount of a composition comprising a population of protocells according to any of embodiments 1-66 which have been adapted to deliver an anticancer agent to a cancer cell in said patient.

### Embodiment 83

A method of treating hepatocellular cancer comprising administering to said patient an effective amount of composition according to any of embodiments 77-82.

### Embodiment 84

A method of treating cancer comprising administering to a patient in need an effective amount of a population of protocells according to any of embodiments 2-66 wherein said DNA plasmid is supercoiled and is adapted to express an anticancer polypeptide and/or RNA, optionally in combination with an effective amount of an additional anticancer agent which is formulated as cargo within said protocells.

### Embodiment 85

The method according to embodiment 84 wherein said anticancer polypeptide is ricin toxin chain-A or diphtheria toxin chain-A.

### Embodiment 86

The method according to embodiment 84 or 85 wherein said RNA is shRNA or siRNA which induces apoptosis of a cancer cell.

### Embodiment 87

The method according to any of embodiments 84-86 wherein said siRNA is selected from the group consisting of s565, s7824 or s10234.

### Embodiment 88

The method according to embodiment 86 wherein said shRNA is a cyclin B1-specific shRNA which induces cell death.

### Embodiment 89

The method according to any of embodiments 84-88 wherein said anticancer agent is selected from the group consisting of everolimus, trabectedin, abraxane, TLK 286, AV-299, DN-101, pazopanib, GSK690693, RTA 744, ON 0910.Na, AZD 6244 (ARRY-142886), AMN-107, TKI-258, GSK461364, AZD 1152, enzastaurin, vandetanib, ARQ-197, MK-0457, MLN8054, PHA-739358, R-763, AT-9263, a FLT-3 inhibitor, a VEGFR inhibitor, an EGFR TK inhibitor, an aurora kinase inhibitor, a PIK-1 modulator, a Bcl-2 inhibitor, an HDAC inhbitor, a c-MET inhibitor, a PARP inhibitor, a Cdk inhibitor, an EGFR TK inhibitor, an IGFR-TK inhibitor, an anti-HGF antibody, a PI3 kinase inhibitors, an AKT inhibitor, a JAK/STAT inhibitor, a checkpoint-1 or 2 inhibitor, a focal adhesion kinase inhibitor, a Map kinase kinase (mek) inhibitor, a VEGF trap antibody, pemetrexed, erlotinib, dasatanib, nilotinib, decatanib, panitumumab, amrubicin, oregovomab, Lep-etu, nolatrexed, azd2171, batabulin, ofatumumab, zanolimumab, edotecarin, tetrandrine, rubitecan, tesmilifene, oblimersen, ticilimumab, ipilimumab, gossypol, Bio 111, 131-I-TM-601, ALT-110, BIO 140, CC 8490, cilengitide, gimatecan, IL13-PE38QQR, INO 1001, IPdR₁ KRX-0402, lucanthone, LY 317615, neuradiab, vitespan, Rta 744, Sdx 102, talampanel, atrasentan, Xr 311, romidepsin, ADS-100380, sunitinib, 5-fluorouracil, vorinostat, etoposide, gemcitabine, doxorubicin, liposomal doxorubicin, 5′-deoxy-5-fluorouridine, vincristine, temozolomide, ZK-304709, seliciclib; PD0325901, AZD-6244, capecitabine, L-Glutamic acid, N-[4-[2-(2-amino-4,7-dihydro-4-oxo-1H-pyrrolo[2,3-d]pyrimidin-5-yl)ethyl]benzoyl]-, disodium salt, heptahydrate, camptothecin, PEG-labeled irinotecan, tamoxifen, toremifene citrate, anastrazole, exemestane, letrozole, DES(diethylstilbestrol), estradiol, estrogen, conjugated estrogen, bevacizumab, IMC-1C11, CHIR-258,); 3-[5-(methylsulfonylpiperadinemethyl)-indolylj-quinolone, vatalanib, AG-013736, AVE-0005, the acetate salt of [D-Ser(Bu t) 6,Azgly 10] (pyro-Glu-His-Trp-Ser-Tyr-D-Ser(Bu t)-Leu-Arg-Pro-Azgly-NH₂ acetate [C₅₉H₈₄N₁₈Oi₄-(C₂H₄O₂)_{X} where x=1 to 2.4], goserelin acetate, leuprolide acetate, triptorelin pamoate, medroxyprogesterone acetate, hydroxyprogesterone caproate, megestrol acetate, raloxifene, bicalutamide, flutamide, nilutamide, megestrol acetate, CP-724714; TAK-165, HKI-272, erlotinib, lapatanib, canertinib, ABX-EGF antibody, erbitux, EKB-569, PKI-166, GW-572016, Ionafarnib, BMS-214662, tipifarnib; amifostine, NVP-LAQ824, suberoyl analide hydroxamic acid, valproic acid, trichostatin A, FK-228, SU11248, sorafenib, KRN951, aminoglutethimide, amsacrine, anagrelide, L-asparaginase, Bacillus Calmette-Guerin (BCG) vaccine, bleomycin, buserelin, busulfan, carboplatin, carmustine, chlorambucil, cisplatin, cladribine, clodronate, cyproterone, cytarabine, dacarbazine, dactinomycin, daunorubicin, diethylstilbestrol, epirubicin, fludarabine, fludrocortisone, fluoxymesterone, flutamide, gemcitabine, gleevac, hydroxyurea, idarubicin, ifosfamide, imatinib, leuprolide, levamisole, lomustine, mechlorethamine, melphalan, 6-mercaptopurine, mesna, methotrexate, mitomycin, mitotane, mitoxantrone, nilutamide, octreotide, oxaliplatin, pamidronate, pentostatin, plicamycin, porfimer, procarbazine, raltitrexed, rituximab, streptozocin, teniposide, testosterone, thalidomide, thioguanine, thiotepa, tretinoin, vindesine, 13-cis-retinoic acid, phenylalanine mustard, uracil mustard, estramustine, altretamine, floxuridine, 5-deooxyuridine, cytosine arabinoside, 6-mecaptopurine, deoxycoformycin, calcitriol, valrubicin, mithramycin, vinblastine, vinorelbine, topotecan, razoxin, marimastat, COL-3, neovastat, BMS-275291, squalamine, endostatin, SU5416, SU6668, EMD121974, interleukin-12, IM862, angiostatin, vitaxin, droloxifene, idoxyfene, spironolactone, finasteride, cimitidine, trastuzumab, denileukin diftitox, gefitinib, bortezimib, paclitaxel, cremophor-free paclitaxel, docetaxel, epithilone B, BMS-247550, BMS-310705, droloxifene, 4-hydroxytamoxifen, pipendoxifene, ERA-923, arzoxifene, fulvestrant, acolbifene, lasofoxifene, idoxifene, TSE-424, HMR-3339, ZK186619, topotecan, PTK787/ZK 222584, VX-745, PD 184352, rapamycin, 40-O-(2-hydroxyethyl)-rapamycin, temsirolimus, AP-23573, RAD001, ABT-578, BC-210, LY294002, LY292223, LY292696, LY293684, LY293646, wortmannin, ZM336372, L-779,450, PEG-filgrastim, darbepoetin, erythropoietin, granulocyte colony-stimulating factor, zolendronate, prednisone, cetuximab, granulocyte macrophage colony-stimulating factor, histrelin, pegylated interferon alfa-2a, interferon alfa-2a, pegylated interferon alfa-2b, interferon alfa-2b, azacitidine, PEG-L-asparaginase, lenalidomide, gemtuzumab, hydrocortisone, interleukin-11, dexrazoxane, alemtuzumab, all-transretinoic acid, ketoconazole, interleukin-2, megestrol, immune globulin, nitrogen mustard, methylprednisolone, ibritgumomab tiuxetan, androgens, decitabine, hexamethylmelamine, bexarotene, tositumomab, arsenic trioxide, cortisone, editronate, mitotane, cyclosporine, liposomal daunorubicin, Edwina-asparaginase, strontium 89, casopitant, netupitant, an NK-1 receptor antagonists, palonosetron, aprepitant, diphenhydramine, hydroxyzine, metoclopramide, lorazepam, alprazolam, haloperidol, droperidol, dronabinol, dexamethasone, methylprednisolone, prochlorperazine, granisetron, ondansetron, dolasetron, tropisetron, pegfilgrastim, erythropoietin, epoetin alfa, darbepoetin alfa and mixtures thereof.

### Embodiment 90

The method according to any of embodiments 77-81 wherein said protocells or said composition outside of said protocells further comprise an antiviral agent.

### Embodiment 91

The method according to embodiment 90 wherein said antiviral agent is an anti-HBV agent or an anti-HCV agent.

### Embodiment 92

A method of treating cancer in a patient comprising administering to a patient in need an effective amount of a composition according to any of embodiments 67-81.

### Embodiment 93

The method according to any of embodiments 92 wherein said cancer is squamous-cell carcinoma, adenocarcinoma, hepatocellular carcinoma, renal cell carcinomas, carcinoma of the bladder, bone, bowel, breast, cervix, colon (colorectal), esophagus, head, kidney, liver (hepatocellular), lung, nasopharyngeal, neck, ovary, testicles, pancreas, prostate, and stomach; a leukemia, Burkitt's lymphoma, Non-Hodgkin's lymphoma, B-cell lymphoma; malignant melanoma; myeloproliferative diseases; Ewing's sarcoma, hemangiosarcoma, Kaposi's sarcoma, liposarcoma, myosarcomas, peripheral neuroepithelioma, synovial sarcoma; gliomas, astrocytomas, oligodendrogliomas, ependymomas, gliobastomas, neuroblastomas, ganglioneuromas, gangliogliomas, medulloblastomas, pineal cell tumors, meningiomas, meningeal sarcomas, neurofibromas, Schwannomas, bowel cancer, breast cancer, prostate cancer, cervical cancer, uterine cancer, non-small cell lung cancer, small cell lung cancer, mixed small cell and non-small cell lung cancer, pleural mesothelioma, pleural mesothelioma, testicular cancer, thyroid cancer and astrocytoma.

### Embodiment 94

A method of diagnosing cancer in a patient at risk for cancer, the method comprising administering to said patient a pharmaceutical composition comprising a population of protocells according to any of embodiments 1-66 comprising targeting peptides adapted to selectively bind to cancer cells and deliver the protocells to said cells, wherein said protocells comprise a plasmid DNA adapted to express a reporter molecule and optionally comprise an additional reporter molecule, whereupon the binding of the protocell to a cancer cell in said patient will release said reporter molecules into the cancer cells, if present, and the reporter molecules will elicit a signal which can be compared with a standard to determine whether or not the patient has cancer and if so, the extent of the cancer and/or size of a cancerous tumor, if present.

### Embodiment 95

A method of monitoring cancer therapy in a patient comprising administering to said patient a population of protocells according to any of embodiments 1-66 comprising targeting peptides adapted to selectively bind to cancer cells and deliver the protocells to said cells, wherein said protocells comprise a plasmid DNA adapted to express a reporter molecule and optionally comprise an additional reporter molecule, whereupon the binding of the protocell to a cancer cell in said patient will release said reporter molecules into the cancer cells and the reporter molecules will elicit a signal which can be compared with a standard at the commencement of therapy and at varying intervals during the course of therapy to determine whether or not patient is responding to the therapy and if so, the extent of the response to the therapy.

### Embodiment 96

A transdermal protocell comprising a plurality of porous nanoparticulates that (a) are loaded with one or more pharmaceutically-active agents and (b) that are encapsulated by and that support a lipid bilayer, wherein the lipid bilayer comprises at least one CD47 molecule or active fragment thereof and one or more stratum corneum permeability-enhancers selected from the group consisting of a monounsaturated omega-9 fatty acid, an alcohol, a diol, a solvent, a co-solvent, R8 peptide, and an edge activator, wherein the protocell has an average diameter of between about 50 nm to about 300 nm.

### Embodiment 97

The transdermal protocell of embodiment 96, wherein the monounsaturated omega-9 fatty acid is selected from the group consisting of oleic acid, elaidic acid, eicosenoic acid, mead acid, erucic acid, and nervonic acid, most preferably oleic acid, and mixtures thereof.

### Embodiment 98

The transdermal protocell of embodiment 96, wherein the alcohol is selected from the group consisting of methanol, ethanol, propanol, and butanol, and mixtures thereof, and the solvent and co-solvent are selected from the group consisting of PEG 400 and DMSO.

### Embodiment 99

The transdermal protocell of embodiment 96, wherein the diol is selected from the group consisting of ethylene glycol and polyethylene glycol, and mixtures thereof.

### Embodiment 100

The transdermal protocell of embodiment 96, wherein the edge activator is selected from the group consisting of bile salts, polyoxyethylene esters and polyoxyethylene ethers, and a single-chain surfactant, and mixtures thereof.

### Embodiment 101

The transdermal protocell of embodiment 96, wherein the edge activator is sodium deoxycholate.

### Embodiment 102

The transdermal protocell of embodiment 97, wherein the protocell has an average diameter of between about 50 nm to about 300 nm.

### Embodiment 103

The transdermal protocell of embodiment 96, wherein the protocell has an average diameter of between about 55 nm to about 270 nm.

### Embodiment 104

The transdermal protocell of embodiment 96, wherein the protocell has an average diameter of between about 60 nm to about 240 nm.

### Embodiment 105

The transdermal protocell of embodiment 96, wherein the protocell has an average diameter of between about 65 nm to about 210 nm.

### Embodiment 106

The transdermal protocell of embodiment 96, wherein the protocell has an average diameter of between about 65 nm to about 190 nm.

### Embodiment 107

The transdermal protocell of embodiment 96, wherein the protocell has an average diameter of between about 65 nm to about 160 nm.

### Embodiment 108

The transdermal protocell of embodiment 96, wherein the protocell has an average diameter of between about 65 nm to about 130 nm.

### Embodiment 109

The transdermal protocell of embodiment 96, wherein the protocell has an average diameter of between about 65 nm to about 100 nm.

### Embodiment 110

The transdermal protocell of embodiment 96, wherein the protocell has an average diameter of between about 65 nm to about 90 nm.

### Embodiment 111

The transdermal protocell of embodiment 96, wherein the protocell has an average diameter of between about, more preferably between about 65 nm to about 80 nm.

### Embodiment 112

The transdermal protocell of embodiment 96, wherein the protocell has an average diameter of between about 65 nm to about 75 nm.

### Embodiment 113

The transdermal protocell of embodiment 96, wherein the protocell has an average diameter of between about 65 nm to about 66, 67, 68, 69, 70, 71, 72, 73, 74 or 75 nm.

### Embodiment 114

The transdermal protocell of embodiment 96, wherein the protocell has an average diameter of around 70 nm.

### Embodiment 115

The transdermal protocell of embodiments 96-114, wherein (a) the nanoparticulates are comprised of one or more compositions selected from the group consisting of silica, a biodegradable polymer, a solgel, a metal and a metal oxide; and (b) the protocell includes at least one anticancer agent.

### Embodiment 116

The transdermal protocell of embodiments 96-115, wherein (a) the nanoparticulates are comprised of one or more compositions selected from the group consisting of silica, a biodegradable polymer, a solgel, a metal and a metal oxide; and (b) the protocell includes at least one anticancer agent selected from the group consisting of everolimus, trabectedin, abraxane, TLK 286, AV-299, DN-101, pazopanib, GSK690693, RTA 744, ON 0910.Na, AZD 6244 (ARRY-142886), AMN-107, TKI-258, GSK461364, AZD 1152, enzastaurin, vandetanib, ARQ-197, MK-0457, MLN8054, PHA-739358, R-763, AT-9263, a FLT-3 inhibitor, a VEGFR inhibitor, an EGFR TK inhibitor, an aurora kinase inhibitor, a PIK-1 modulator, a Bcl-2 inhibitor, an HDAC inhbitor, a c-MET inhibitor, a PARP inhibitor, a Cdk inhibitor, an EGFR TK inhibitor, an IGFR-TK inhibitor, an anti-HGF antibody, a PI3 kinase inhibitors, an AKT inhibitor, a JAK/STAT inhibitor, a checkpoint-1 or 2 inhibitor, a focal adhesion kinase inhibitor, a Map kinase kinase (mek) inhibitor, a VEGF trap antibody, pemetrexed, erlotinib, dasatanib, nilotinib, decatanib, panitumumab, amrubicin, oregovomab, Lep-etu, nolatrexed, azd2171, batabulin, ofatumumab, zanolimumab, edotecarin, tetrandrine, rubitecan, tesmilifene, oblimersen, ticilimumab, ipilimumab, gossypol, Bio 111, 131-I-TM-601, ALT-110, BIO 140, CC 8490, cilengitide, gimatecan, IL13-PE38QQR, NO 1001, IPdR₁ KRX-0402, lucanthone, LY 317615, neuradiab, vitespan, Rta 744, Sdx 102, talampanel, atrasentan, Xr 311, romidepsin, ADS-100380, sunitinib, 5-fluorouracil, vorinostat, etoposide, gemcitabine, doxorubicin, liposomal doxorubicin, 5′-deoxy-5-fluorouridine, vincristine, temozolomide, ZK-304709, seliciclib; PD0325901, AZD-6244, capecitabine, L-Glutamic acid, N-[4-[2-(2-amino-4,7-dihydro-4-oxo-1H-pyrrolo[2,3-d]pyrimidin-5-yl)ethyl]benzoyl]-, disodium salt, heptahydrate, camptothecin, PEG-labeled irinotecan, tamoxifen, toremifene citrate, anastrazole, exemestane, letrozole, DES(diethylstilbestrol), estradiol, estrogen, conjugated estrogen, bevacizumab, IMC-1C11, CHIR-258,); 3-[5-(methylsulfonylpiperadinemethyl)-indolylj-quinolone, vatalanib, AG-013736, AVE-0005, the acetate salt of [D-Ser(Bu t) 6,Azgly 10] (pyro-Glu-His-Trp-Ser-Tyr-D-Ser(Bu t)-Leu-Arg-Pro-Azgly-NH₂ acetate [C₅₉H₈₄N₁₈Oi₄-(C₂H₄O₂)_{X} where x=1 to 2.4], goserelin acetate, leuprolide acetate, triptorelin pamoate, medroxyprogesterone acetate, hydroxyprogesterone caproate, megestrol acetate, raloxifene, bicalutamide, flutamide, nilutamide, megestrol acetate, CP-724714; TAK-165, HKI-272, erlotinib, lapatanib, canertinib, ABX-EGF antibody, erbitux, EKB-569, PKI-166, GW-572016, Ionafarnib, BMS-214662, tipifarnib; amifostine, NVP-LAQ824, suberoyl analide hydroxamic acid, valproic acid, trichostatin A, FK-228, SU11248, sorafenib, KRN951, aminoglutethimide, amsacrine, anagrelide, L-asparaginase, Bacillus Calmette-Guerin (BCG) vaccine, bleomycin, buserelin, busulfan, carboplatin, carmustine, chlorambucil, cisplatin, cladribine, clodronate, cyproterone, cytarabine, dacarbazine, dactinomycin, daunorubicin, diethylstilbestrol, epirubicin, fludarabine, fludrocortisone, fluoxymesterone, flutamide, gemcitabine, gleevac, hydroxyurea, idarubicin, ifosfamide, imatinib, leuprolide, levamisole, lomustine, mechlorethamine, melphalan, 6-mercaptopurine, mesna, methotrexate, mitomycin, mitotane, mitoxantrone, nilutamide, octreotide, oxaliplatin, pamidronate, pentostatin, plicamycin, porfimer, procarbazine, raltitrexed, rituximab, streptozocin, teniposide, testosterone, thalidomide, thioguanine, thiotepa, tretinoin, vindesine, 13-cis-retinoic acid, phenylalanine mustard, uracil mustard, estramustine, altretamine, floxuridine, 5-deooxyuridine, cytosine arabinoside, 6-mecaptopurine, deoxycoformycin, calcitriol, valrubicin, mithramycin, vinblastine, vinorelbine, topotecan, razoxin, marimastat, COL-3, neovastat, BMS-275291, squalamine, endostatin, SU5416, SU6668, EMD121974, interleukin-12, IM862, angiostatin, vitaxin, droloxifene, idoxyfene, spironolactone, finasteride, cimitidine, trastuzumab, denileukin diftitox, gefitinib, bortezimib, paclitaxel, cremophor-free paclitaxel, docetaxel, epithilone B, BMS-247550, BMS-310705, droloxifene, 4-hydroxytamoxifen, pipendoxifene, ERA-923, arzoxifene, fulvestrant, acolbifene, lasofoxifene, idoxifene, TSE-424, HMR-3339, ZK186619, topotecan, PTK787/ZK 222584, VX-745, PD 184352, rapamycin, 40-O-(2-hydroxyethyl)-rapamycin, temsirolimus, AP-23573, RAD001, ABT-578, BC-210, LY294002, LY292223, LY292696, LY293684, LY293646, wortmannin, ZM336372, L-779,450, PEG-filgrastim, darbepoetin, erythropoietin, granulocyte colony-stimulating factor, zolendronate, prednisone, cetuximab, granulocyte macrophage colony-stimulating factor, histrelin, pegylated interferon alfa-2a, interferon alfa-2a, pegylated interferon alfa-2b, interferon alfa-2b, azacitidine, PEG-L-asparaginase, lenalidomide, gemtuzumab, hydrocortisone, interleukin-11, dexrazoxane, alemtuzumab, all-transretinoic acid, ketoconazole, interleukin-2, megestrol, immune globulin, nitrogen mustard, methylprednisolone, ibritgumomab tiuxetan, androgens, decitabine, hexamethylmelamine, bexarotene, tositumomab, arsenic trioxide, cortisone, editronate, mitotane, cyclosporine, liposomal daunorubicin, Edwina-asparaginase, strontium 89, casopitant, netupitant, an NK-1 receptor antagonists, palonosetron, aprepitant, diphenhydramine, hydroxyzine, metoclopramide, lorazepam, alprazolam, haloperidol, droperidol, dronabinol, dexamethasone, methylprednisolone, prochlorperazine, granisetron, ondansetron, dolasetron, tropisetron, pegfilgrastim, erythropoietin, epoetin alfa, darbepoetin alfa and mixtures thereof.

### Embodiment 117

A transdermal protocell comprising a plurality of porous nanoparticulates that (a) are loaded with a pharmaceutically-effective amount of imatinib and (b) that are encapsulated by and that support a lipid bilayer, wherein the lipid bilayer comprises at least one CD47 molecule or an active fragment thereof and one or more stratum corneum permeability-enhancers selected from the group consisting of PEG 400, DMSO and ethanol, and mixtures thereof, and wherein the protocell has an average diameter of between about 65 nm to about 66, 67, 68, 69, 70, 71, 72, 73, 74 or 75 nm.

### Embodiment 118

The transdermal protocell of embodiment 117, wherein the protocell has an average flux of imatinib of around 0.20 to about 0.30 μg/cm² hr.

### Embodiment 119

The transdermal protocell of embodiment 117, wherein the lipid bilayer is comprised of lipids selected from the group consisting of 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-dioleoyl-sn-glycero-3-[phosphor-L-serine] (DOPS), 1,2-dioleoyl-3-trimethylammonium-propane (18:1 DOTAP), 1,2-dioleoyl-sn-glycero-3-phospho-(1′-rac-glycerol) (DOPG), 1,2-dioleoyl-sn-glycero-3-phosphoethanolamine (DOPE), 1,2-dipalmitoyl-sn-glycero-3-phosphoethanolamine (DPPE), 1,2-dioleoyl-sn-glycero-3-phosphoethanolamine-N-[methoxy(polyethylene glycol)-2000] (18:1 PEG-2000 PE), 1,2-dipalmitoyl-sn-glycero-3-phosphoethanolamine-N-[methoxy(polyethylene glycol)-2000] (16:0 PEG-2000 PE), 1-Oleoyl-2-[12-[(7-nitro-2-1,3-benzoxadiazol-4-yl)amino]lauroyl]-sn-Glycero-3-Phosphocholine (18:1-12:0 NBD PC), 1-palmitoyl-2-{12-[(7-nitro-2-1,3-benzoxadiazol-4-yl)amino]lauroyl}-sn-glycero-3-phosphocholine (16:0-12:0 NBD PC), cholesterol and mixtures thereof.

### Embodiment 120

A method of treating a subject who suffers from a cancer, the method comprising transdermally administering to the subject a pharmaceutically-effective amount of a protocell of embodiments 110-114.

### Embodiment 121

A method of treating a subject who suffers from one or more diseases selected from the group consisting of chronic myelogenous leukemia, a gastrointestinal stromal tumor and acute lymphocytic leukemia hypereosinophilic syndrome (HES), the method comprising transdermally administering to the subject a pharmaceutically-effective amount of a protocell of embodiments 117-119.

### Embodiment 122

A method of treating a subject who suffers from a cancer, the method comprising transdermally administering to the subject a pharmaceutically-effective amount of a protocell of embodiment 121.

### Embodiment 123

A transdermal pharmaceutical composition comprising a pharmaceutically-effective amount of a protocell of embodiments 96-114, 117, 118 and 119, and optionally a pharmaceutically-acceptable excipient.

### Embodiment 124

A transdermal pharmaceutical composition comprising a pharmaceutically-effective amount of a protocell of embodiment 115, and optionally a pharmaceutically-acceptable excipient.

### Embodiment 125

A transdermal pharmaceutical composition comprising a pharmaceutically-effective amount of a protocell of embodiment 116, and optionally a pharmaceutically-acceptable excipient.

### Embodiment 126

A protocell comprising a plurality of negatively-charged, nanoporous, nanoparticulate silica cores that are modified with an amine-containing silane (AEPTMS) and that (a) are loaded with a siRNA or ricin toxin A-chain and (b) that are encapsulated by and that support a lipid bilayer comprising one of more lipids selected from the group consisting of 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-dioleoyl-sn-glycero-3-[phosphor-L-serine] (DOPS), 1,2-dioleoyl-3-trimethylammonium-propane (18:1 DOTAP), 1,2-dioleoyl-sn-glycero-3-phospho-(1′-rac-glycerol) (DOPG), 1,2-dioleoyl-sn-glycero-3-phosphoethanolamine (DOPE), 1,2-dipalmitoyl-sn-glycero-3-phosphoethanolamine (DPPE), 1,2-dioleoyl-sn-glycero-3-phosphoethanolamine-N-[methoxy(polyethylene glycol)-2000] (18:1 PEG-2000 PE), 1,2-dipalmitoyl-sn-glycero-3-phosphoethanolamine-N-[methoxy(polyethylene glycol)-2000] (16:0 PEG-2000 PE), 1-Oleoyl-2-[12-[(7-nitro-2-1,3-benzoxadiazol-4-yl)amino]lauroyl]-sn-Glycero-3-Phosphocholine (18:1-12:0 NBD PC), 1-palmitoyl-2-{12-[(7-nitro-2-1,3-benzoxadiazol-4-yl)amino]lauroyl}-sn-glycero-3-phosphocholine (16:0-12:0 NBD PC), cholesterol and mixtures/combinations thereof, wherein the lipid bilayer comprises a cationic lipid and one or more zwitterionic phospholipids and at least one CD47 molecule or an active fragment thereof.

### Embodiment 127

The protocell of embodiment 126, wherein the lipid is selected from the group consisting of 1,2-dioleoyl-3-trimethylammonium-propane (18:1 DOTAP) or 1,2-dioleoyl-sn-glycero-3-phospho-(1′-rac-glycerol) (DOPG), 1,2-dioleoyl-sn-glycero-3-phosphoethanolamine (DOPE) and mixtures thereof.

### Embodiment 128

The protocell of embodiment 127, wherein the protocell has at least one of the following characteristics: a BET surface area of greater than about 600 m²/g, a pore volume fraction of between about 60% to about 70%, a multimodal pore morphology composed of pores having an average diameter of between about 20 nm to about 30 nm, and surface-accessible pores interconnected by pores having an average diameter of between about 5 nm to about 15 nm.

### Embodiment 129

The protocell of embodiments 127 or 128, wherein the protocell encapsulates around 10 nM of siRNA per 10¹⁰ nanoparticulate silica cores.

### Embodiment 130

A protocell comprising a plurality of negatively-charged, nanoporous, nanoparticulate silica cores that are modified with an amine-containing silane (AEPTMS) and that:

(a) are loaded with one or more siRNAs that target members of the cyclin superfamily selected from the group consisting of cyclin A2, cyclin B1, cyclin D1, and cyclin E; and

(b) that are encapsulated by and that support a lipid bilayer comprising one of more lipids selected from the group consisting of 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-dioleoyl-sn-glycero-3-[phosphor-L-serine] (DOPS), 1,2-dioleoyl-3-trimethylammonium-propane (18:1 DOTAP), 1,2-dioleoyl-sn-glycero-3-phospho-(1′-rac-glycerol) (DOPG), 1,2-dioleoyl-sn-glycero-3-phosphoethanolamine (DOPE), 1,2-dipalmitoyl-sn-glycero-3-phosphoethanolamine (DPPE), 1,2-dioleoyl-sn-glycero-3-phosphoethanolamine-N-[methoxy(polyethylene glycol)-2000] (18:1 PEG-2000 PE), 1,2-dipalmitoyl-sn-glycero-3-phosphoethanolamine-N-[methoxy(polyethylene glycol)-2000] (16:0 PEG-2000 PE), 1-Oleoyl-2-[12-[(7-nitro-2-1,3-benzoxadiazol-4-yl)amino]lauroyl]-sn-Glycero-3-Phosphocholine (18:1-12:0 NBD PC), 1-palmitoyl-2-{12-[(7-nitro-2-1,3-benzoxadiazol-4-yl)amino]lauroyl}-sn-glycero-3-phosphocholine (16:0-12:0 NBD PC), cholesterol and mixtures/combinations thereof,

and wherein (1) the lipid bilayer is loaded with SP94 and an endosomolytic peptide, and (2) the protocell selectively binds to a hepatocellular carcinoma cell.

### Embodiment 131

The protocell of embodiment 129, wherein the lipid bilayer comprises DOPC/DOPE/cholesterol/PEG-2000 in an approximately 55:5:30:10 mass ratio.

### Embodiment 132

A method of treating a subject suffering from a cancer comprising administering to the subject a pharmaceutically-effective amount of a protocell of embodiments 126-131.

### Embodiment 133

The method of embodiment 132, wherein the subject suffers from liver cancer and is administered a pharmaceutically-effective amount of a protocell of embodiments 126-131.

### Embodiment 134

A pharmaceutical composition comprising a pharmaceutically-effective amount of a protocell of embodiments 126-131, and optionally a pharmaceutically-acceptable excipient.

### Embodiment 135

A protocell comprising a plurality of mesoporous, nanoparticulate silica cores that (a) are loaded with a siRNA that induces sequence-specific degradation of NiV nucleocapsid protein (NiV-N) mRNA and (b) that are encapsulated by and that support a lipid bilayer comprising at least one CD47 molecule or an active fragment thereof and one of more lipids selected from the group consisting of 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-dioleoyl-sn-glycero-3-[phosphor-L-serine] (DOPS), 1,2-dioleoyl-3-trimethylammonium-propane (18:1 DOTAP), 1,2-dioleoyl-sn-glycero-3-phospho-(1′-rac-glycerol) (DOPG), 1,2-dioleoyl-sn-glycero-3-phosphoethanolamine (DOPE), 1,2-dipalmitoyl-sn-glycero-3-phosphoethanolamine (DPPE), 1,2-dioleoyl-sn-glycero-3-phosphoethanolamine-N-[methoxy(polyethylene glycol)-2000] (18:1 PEG-2000 PE), 1,2-dipalmitoyl-sn-glycero-3-phosphoethanolamine-N-[methoxy(polyethylene glycol)-2000] (16:0 PEG-2000 PE), 1-Oleoyl-2-[12-[(7-nitro-2-1,3-benzoxadiazol-4-yl)amino]lauroyl]-sn-Glycero-3-Phosphocholine (18:1-12:0 NBD PC), 1-palmitoyl-2-{12-[(7-nitro-2-1,3-benzoxadiazol-4-yl)amino]lauroyl}-sn-glycero-3-phosphocholine (16:0-12:0 NBD PC), cholesterol and mixtures/combinations thereof.

### Embodiment 136

The protocell of embodiment 135, wherein the lipid bilayer comprises 1,2-dioleoyl-sn-glycero-3-phosphocholine (DOPC), 1,2-dioleoyl-sn-glycero-3-phosphoethanolamine (DOPE) a polyethylene glycol (PEG), a targeting peptide, and R8, and wherein the mesoporous, nanoparticulate silica cores (1) each have an average diameter of around 100 nm, an average surface area of greater than 1,000 m²/g and surface-accessible pores having an average diameter of between about 20 nm to about 25 nm, and (2) have a siRNA load of around 1 μM per 10¹⁰ particles or greater.

### Embodiment 137

The protocell of embodiment 136, wherein the targeting peptide is a peptide that binds to ephrin B2 (EB2).

### Embodiment 138

The protocell of embodiment 137, wherein the targeting peptide is TGAILHP (SEQ ID NO:18).

### Embodiment 139

The protocell of embodiment 138, wherein the protocell comprises around 0.01 to around 0.02 wt % of TGAILHP (SEQ ID NO:18) around 10 wt % PEG-2000 and around 0.500 wt % of R8.

### Embodiment 140

A method of treating a subject who has been infected by, or is at risk of infection with, Nipah virus (NiV), the method comprising administering to the subject a pharmaceutically-effective amount of a protocell of embodiments 135-139.

### Embodiment 141

A pharmaceutical composition comprising a pharmaceutically-effective amount of a protocell of embodiments 135-139, and optionally a pharmaceutically-acceptable excipient.

### Embodiment 142

A protocell comprising a plurality of negatively-charged, nanoporous, nanoparticulate silica cores that:

(a) are modified with an amine-containing silane selected from the group consisting of (1) a primary amine, a secondary amine a tertiary amine, each of which is functionalized with a silicon atom (2) a monoamine or a polyamine (3) N-(2-aminoethyl)-3-aminopropyltrimethoxysilane (AEPTMS) (4) 3-aminopropyltrimethoxysilane (APTMS) (5) 3-aminopropyltriethoxysilane (APTS) (6) an amino-functional trialkoxysilane, and (7) protonated secondary amines, protonated tertiary alkyl amines, protonated amidines, protonated guanidines, protonated pyridines, protonated pyrimidines, protonated pyrazines, protonated purines, protonated imidazoles, protonated pyrroles, and quaternary alkyl amines, or combinations thereof;

(b) are loaded with a siRNA or ricin toxin A-chain; and

(c) that are encapsulated by and that support a lipid bilayer comprising at least one CD47 molecule or an active fragment thereof and one of more lipids selected from the group consisting of 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-dioleoyl-sn-glycero-3-[phosphor-L-serine] (DOPS), 1,2-dioleoyl-3-trimethylammonium-propane (18:1 DOTAP), 1,2-dioleoyl-sn-glycero-3-phospho-(1′-rac-glycerol) (DOPG), 1,2-dioleoyl-sn-glycero-3-phosphoethanolamine (DOPE), 1,2-dipalmitoyl-sn-glycero-3-phosphoethanolamine (DPPE), 1,2-dioleoyl-sn-glycero-3-phosphoethanolamine-N-[methoxy(polyethylene glycol)-2000] (18:1 PEG-2000 PE), 1,2-dipalmitoyl-sn-glycero-3-phosphoethanolamine-N-[methoxy(polyethylene glycol)-2000] (16:0 PEG-2000 PE), 1-Oleoyl-2-[12-[(7-nitro-2-1,3-benzoxadiazol-4-yl)amino]lauroyl]-sn-Glycero-3-Phosphocholine (18:1-12:0 NBD PC), 1-palmitoyl-2-{12-[(7-nitro-2-1,3-benzoxadiazol-4-yl)amino]lauroyl}-sn-glycero-3-phosphocholine (16:0-12:0 NBD PC), cholesterol and mixtures/combinations thereof, and wherein the lipid bilayer comprises a cationic lipid and one or more zwitterionic phospholipids.

### Embodiment 143

The protocell of embodiment 142, wherein the lipid is selected from the group consisting of 1,2-dioleoyl-3-trimethylammonium-propane (18:1 DOTAP) or 1,2-dioleoyl-sn-glycero-3-phospho-(1′-rac-glycerol) (DOPG), 1,2-dioleoyl-sn-glycero-3-phosphoethanolamine (DOPE) and mixtures thereof.

### Embodiment 144

The protocell of embodiment 143, wherein the protocell has at least one of the following characteristics: a BET surface area of greater than about 600 m²/g, a pore volume fraction of between about 60% to about 70%, a multimodal pore morphology composed of pores having an average diameter of between about 20 nm to about 30 nm, surface-accessible pores interconnected by pores having an average diameter of between about 5 nm to about 15 nm.

### Embodiment 145

The protocell of embodiments 143 or 144, wherein the protocell encapsulates around 10 nM of siRNA per 10¹⁰ nanoparticulate silica cores.

### Embodiment 146

A protocell comprising a plurality of negatively-charged, nanoporous, nanoparticulate silica cores that:

(a) are modified with an amine-containing silane selected from the group consisting of (1) a primary amine, a secondary amine a tertiary amine, each of which is functionalized with a silicon atom (2) a monoamine or a polyamine (3) N-(2-aminoethyl)-3-aminopropyltrimethoxysilane (AEPTMS) (4) 3-aminopropyltrimethoxysilane (APTMS) (5) 3-aminopropyltriethoxysilane (APTS) (6) an amino-functional trialkoxysilane, and (7) protonated secondary amines, protonated tertiary alkyl amines, protonated amidines, protonated guanidines, protonated pyridines, protonated pyrimidines, protonated pyrazines, protonated purines, protonated imidazoles, protonated pyrroles, and quaternary alkyl amines, or combinations thereof;

(b) are loaded with one or more siRNAs that target members of the cyclin superfamily selected from the group consisting of cyclin A2, cyclin B1, cyclin D1, and cyclin E; and

(c) that are encapsulated by and that support a lipid bilayer comprising at least one CD47 molecule or an active fragment thereof and one of more lipids selected from the group consisting of 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-dioleoyl-sn-glycero-3-[phosphor-L-serine] (DOPS), 1,2-dioleoyl-3-trimethylammonium-propane (18:1 DOTAP), 1,2-dioleoyl-sn-glycero-3-phospho-(1′-rac-glycerol) (DOPG), 1,2-dioleoyl-sn-glycero-3-phosphoethanolamine (DOPE), 1,2-dipalmitoyl-sn-glycero-3-phosphoethanolamine (DPPE), 1,2-dioleoyl-sn-glycero-3-phosphoethanolamine-N-[methoxy(polyethylene glycol)-2000] (18:1 PEG-2000 PE), 1,2-dipalmitoyl-sn-glycero-3-phosphoethanolamine-N-[methoxy(polyethylene glycol)-2000] (16:0 PEG-2000 PE), 1-Oleoyl-2-[12-[(7-nitro-2-1,3-benzoxadiazol-4-yl)amino]lauroyl]-sn-Glycero-3-Phosphocholine (18:1-12:0 NBD PC), 1-palmitoyl-2-{12-[(7-nitro-2-1,3-benzoxadiazol-4-yl)amino]lauroyl}-sn-glycero-3-phosphocholine (16:0-12:0 NBD PC), cholesterol and mixtures/combinations thereof,

and wherein (1) the lipid bilayer is loaded with SP94 and an endosomolytic peptide, and (2) the protocell selectively binds to a hepatocellular carcinoma cell.

### Embodiment 147

The protocell of embodiment 146, wherein the lipid bilayer comprises DOPC/DOPE/cholesterol/PEG-2000 in an approximately 55:5:30:10 mass ratio.

### Embodiment 148

A method of treating a subject suffering from a cancer comprising administering to the subject a pharmaceutically-effective amount of a protocell of embodiments 142-147.

### Embodiment 149

The method of embodiment 148, wherein the subject suffers from liver cancer and is administered a pharmaceutically-effective amount of a protocell of embodiments 146 or 147.

### Embodiment 150

A pharmaceutical composition comprising a pharmaceutically-effective amount of a protocell of embodiments 142-147, and optionally a pharmaceutically-acceptable excipient.

The following non-limiting examples are illustrative of the invention and its advantageous properties, and are not to be taken as limiting the disclosure or claims in any way. In the examples, as well as elsewhere in this application, all parts and percentages are by weight unless otherwise indicated.

### Example 1

One or more of the previously described protocell compositions may be modified to containing lipid which is conjugated to a CD47 molecule and fused to the surface of the protocell. Alternatively, the lipid bilayer described for each of the protocell compositions above, may be modified by incorporating cellular plasma membrane (which contain CD47) and fusing the mixture onto the protocell. The incorporation of CD47 molecules will enhance bioavailability and biodistribution and increase residence time of the CD47 modified protocells inasmuch as the CD47 modified protocells will exhibit much less interaction with immune cells in vivo.

### Example 2—CD47 Conjugation to Protocells Utilizing Heterobifunctional Crosslinkers EDC and Sulfo-NHS

Heterobifunctional linkers ethyl(dimethylaminopropyl) carbodiimide (EDC) and N-Hydroxylsulfosuccinimide (Sulfo-NHS) were utilized to attach rat CD47 protein (available form Creative BioMart) to an amine functionalized lipid incorporated into the supported lipid bilayer of the protocell. The created protocells show relative mondispersity as evidenced by DLS measurements and PDI of the protocells both before and after CD47 addition. As a comparison, size and PDI of liposomes used to make the protocells, the protocells without CD47, and protocells conjugated to CD47 were measured (Table 1).

TABLE 1
DLS measurements of liposomes, protocells, and protocells with CD47
conjugated using EDC and sulfo-NHS.

| Liposomes | | Protocells | | Protocells + CD47 | |
|---|---|---|---|---|---|
| Average Size | PDI | Average Size | PDI | Average Size | PDI |
| 76.13 nm | 0.187 | 157.2 nm | 0.115 | 186.9 nm | 0.102 |

### Example 3—CD47 Conjugation to Protocells Utilizing Heterobifunctional Crosslinker SM-PEG₁₂

SM-PEG₁₂ was utilized to attach CD47 protein to an amine functionalized lipid incorporated into the supported lipid bilayer of the protocell. The created protocells show relative mondispersity as evidenced by DLS measurements and PDI of the protocells both before and after CD47 addition. As a comparison, size and PDI of liposomes used to make the protocells, the protocells without CD47, and protocells conjugated to CD47 were measured (Table 2).

TABLE 2
DLS measurements of liposomes, protocells, and protocells with CD47
conjugated using SM-PEG₁₂.

| Liposomes | | Protocells | | Protocells + CD47 | |
|---|---|---|---|---|---|
| Average Size | PDI | Average Size | PDI | Average Size | PDI |
| 76.96 nm | 0.170 | 170.5 nm | 0.081 | 156.4 nm | 0.070 |

### Example 4—CD47 Conjugation to Protocells Using Click Chemistry Crosslinker

Click chemistry linker propargyl-PEG-maleimide was utilized to attach CD47 protein to an azide functionalized lipid incorporated into the supported lipid bilayer of the protocell. This chemistry utilizes internal cysteine residues in the CD47 protein. The created protocells show relative mondispersity as evidenced by DLS measurements and PDI of the protocells both before and after CD47 addition. As a comparison, size and PDI of liposomes used to make the protocells, the protocells without CD47, and protocells conjugated to CD47 were measured (Table 3).

TABLE 3
DLS measurements of liposomes, protocells, and protocells with CD47
conjugated using propargyl-PEG-maleimide.

| Liposomes | | Protocells | | Protocells + CD47 | |
|---|---|---|---|---|---|
| Average Size | PDI | Average Size | PDI | Average Size | PDI |
| 85.68 nm | 0.207 | 154.6 nm | 0.107 | 147.4 nm | 0.079 |

### Example 5—CD47 Conjugation to Protocells Utilizing NTA/NI Chelate Lipids

DOGS-NTA/Ni is a functional lipids with a NTA/Ni functional group incorporated into the supported lipid bilayer of the protocell. The chelating lipid was then associated with CD47, which had been functionalized with a His tag. The created protocells show relative mondispersity as evidenced by DLS measurements and PDI of the protocells both before and after CD47 addition. As a comparison, size and PDI of liposomes used to make the protocells, the protocells without CD47, and protocells conjugated to CD47 were measured (Table 4).

TABLE 4
DLS measurements of liposomes, protocells, and protocells with
His-tagged CD47 conjugated using DOGS-NTA/Ni.

| Liposomes | | Protocells | | Protocells + CD47 | |
|---|---|---|---|---|---|
| Average Size | PDI | Average Size | PDI | Average Size | PDI |
| 95.39 nm | 0.195 | 119.8 nm | 0.060 | 129.2 nm | 0.089 |

### Example 6—CD47 Conjugation to Protocells Limits Macrophage Uptake

Protocells conjugated to CD47 using each of the methods described in Examples 2-5 were mixed with macrophages to determine if CD47 attached to the lipid bilayer of the protocell limits macrophage phagocytosis. To compare, bare nanoparticles (i.e., not surrounded by a lipid bilayer) and protocells conjugated to PEG were also mixed with macrophages to test for phagocytosis. PEG attachment to nanoparticles has been previously used to limit macrophage phagocytosis.

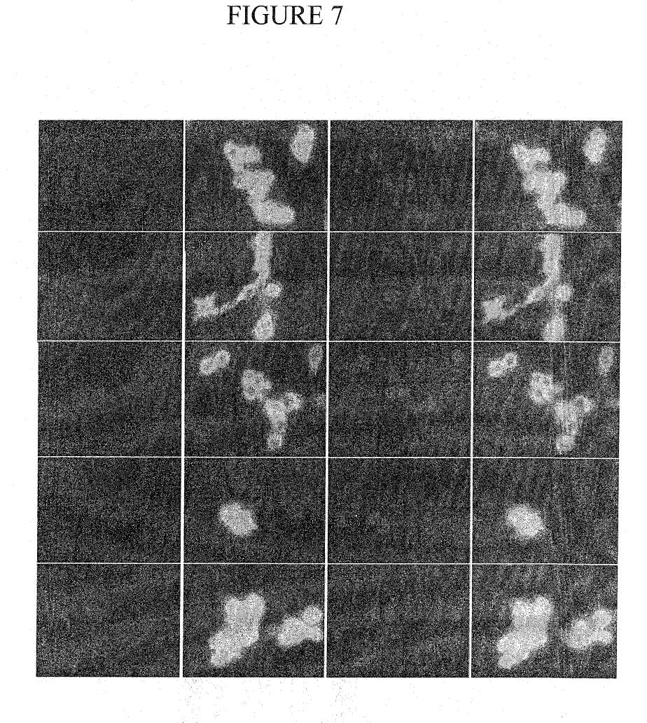

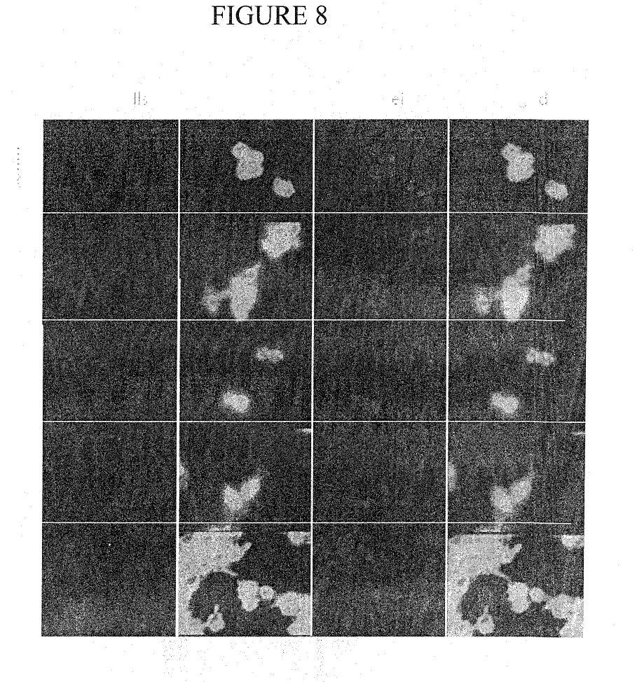

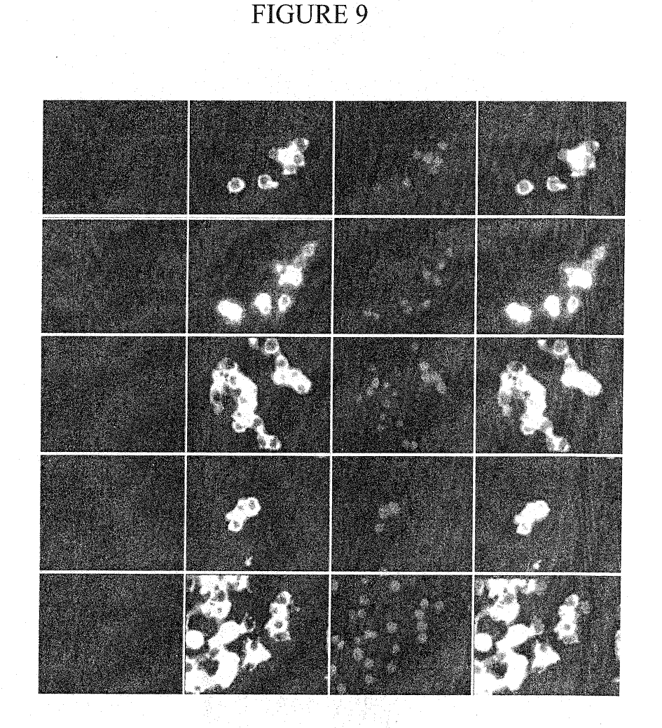

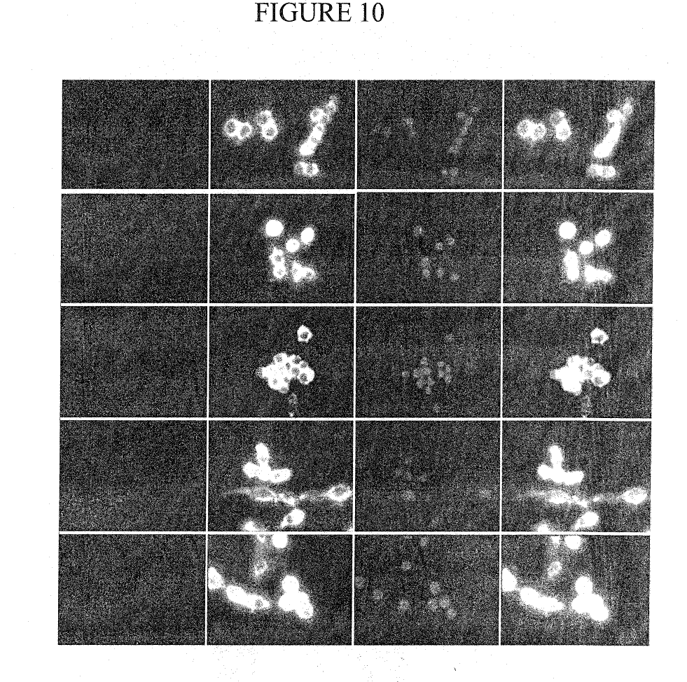

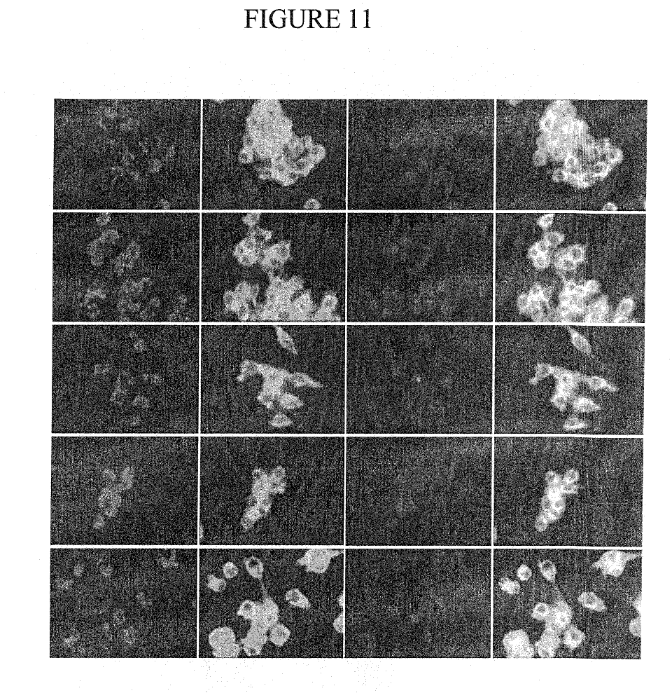

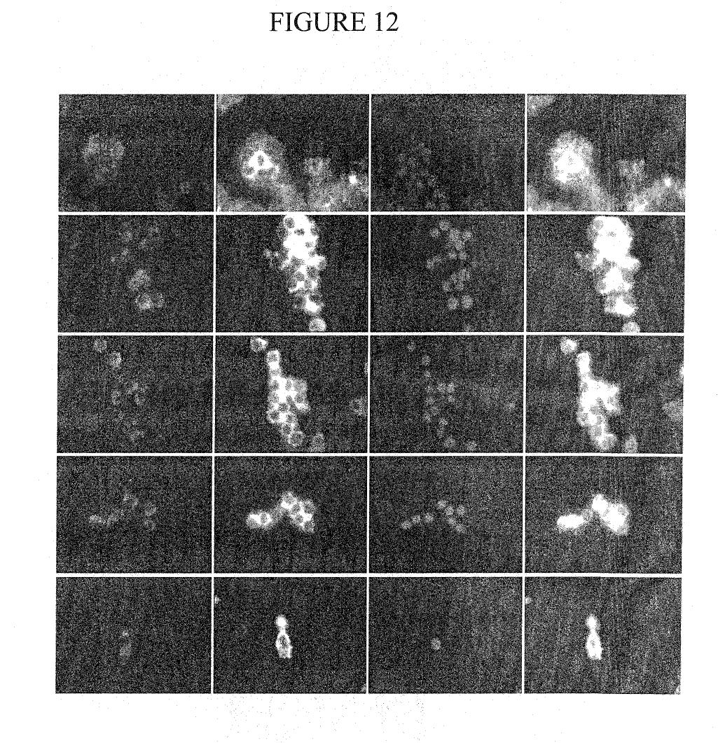

Conjugation of CD47 to the surface of the protocells utilizing any of the methods disclosed in Examples 2-5 results in near elimination of phagocytosis by macrophages. As seen in FIG. 7 (using EDC and Sulfo-NHS as in Example 2), FIG. 8 (using SM-PEG₁₂ as in Example 3), FIG. 9 (using propargyl-PEG-maleimide as in Example 4), and FIG. 10 (using DOGS-NTA/Ni as in Example 5), each method of conjugating CD47 to the protocell lipid bilayer results in limited phagocytosis activity by macrophages after 24 hours. In contrast, bare nanoparticles (FIG. 11) and protocells conjugated to PEG (FIG. 12) were uptaken by macrophages in as little as 30 minutes. In FIGS. 7-12, the protocell cores (or bare nanoparticles) were fluorescently labeled using a red dye, the cytoskeleton of the macrophages was died using phalloidin, and the nuclei of the macrophages were dyed using Hoescht stain. Co-localization of the fluorescently labeled protocell cores (or bare nanoparticles) with the macrophage cytoskeleton indicated uptake of the particles.

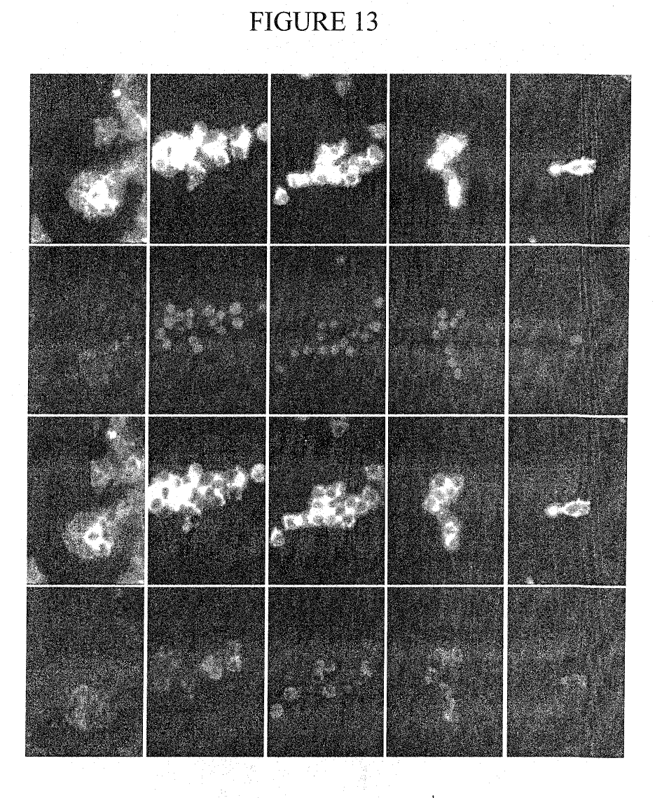

To confirm that the reduction in uptake by macrophages is due to the presence of CD47 on the surface of the protocells, CD47 conjugated protocells were incubated with an anti-CD47 antibody prior to addition to macrophages. The addition of the anti-CD47 antibody effectively blocked the CD47 on the surface of the protocell and resulted in uptake of the protocells by macrophages (FIG. 13).

Uptake of the protocells or bare nanoparticles can also be quantitated by measuring the mean fluorescence intensity of the macrophages utilizing flow cytometry. Macrophages were incubated with fluorescently labeled bare mesoporous silica nanoparticles, protocells with CD47 conjugated to the lipid bilayer using the methods described in Examples 2-5 with a fluorescently labeled core, or protocells with CD47 conjugated to the lipid bilayer using the methods described in Examples 2-5 that were pre-incubated with anti-CD47 antibody. As a control, mean fluorescence intensity of macrophages without being incubated with any particles was also measured.

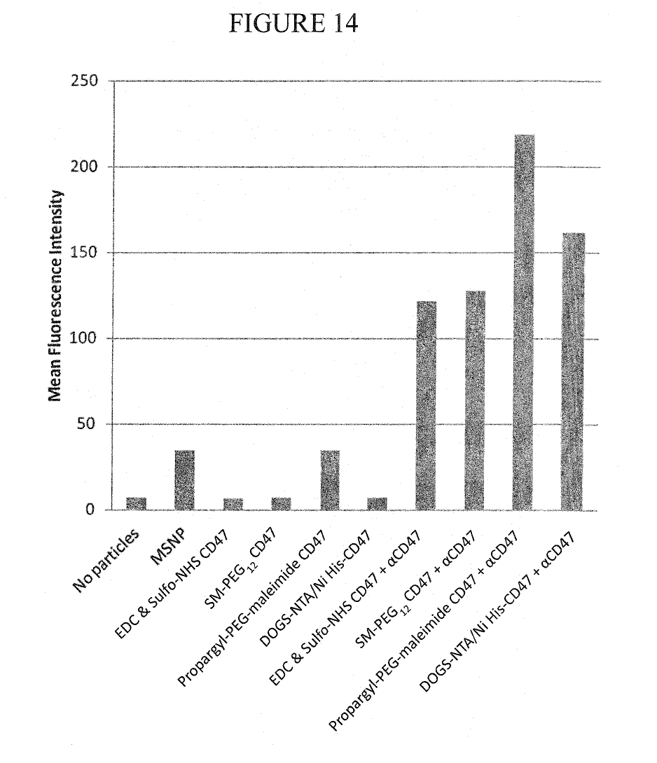

Conjugation of CD47 to protocells using EDC/Sulfo-NHS, SM-PEG₁₂, or the chelating lipid resulted in limiting macrophage phagocytosis of the protocells. Incubation of the protocells with the anti-CD47 antibody resulted in a significant increase in macrophage phagocytosis due to the antibody blocking the CD47 activity. The bare nanoparticles were also uptaken by the macrophages as expected. Protocells conjugated to CD47 using the click chemistry linker as described in Example 5 also resulted in some macrophage phagocytosis of the protocells, likely due to the use of active cysteine residues in the CD47 polypeptide for crosslinking, which could affect the function of the CD47 molecule. These results are shown in FIG. 14.

### Example 7—CD47 Density on Protocells

Protocells samples were conjugated to varying amounts of CD47 before being incubated with macrophages. The CD47 was conjugated to the protocells by incorporating DOGS-NTA/Ni in the protocell lipid bilayer and incubating the protocells with His-tagged CD47. The amount of CD47 conjugated the protocells was controlled by varying the concentration of CD47 during incubation, as shown in Table 5.

TABLE 5
Protocells prepared with varying numbers of CD47 molecules
on the surface

| | Approximate | | |
|---|---|---|---|
| | Number of CD47 | | |
| | Molecules per | | |
| [CD47] | Protocell | Average size | PDI |
| 33 μg/mL | 556 | 129.2 nm | 0.086 |
| 10 μg/mL | 169 | 127.5 | 0.077 |
| 5 μg/mL | 84 | 121.9 | 0.084 |
| 2.5 μg/mL | 42 | 126.8 | 0.071 |
| 1.25 μg/mL | 21 | 124.5 | 0.054 |
| 0.63 μg/mL | 11 | 116.3 | 0.061 |

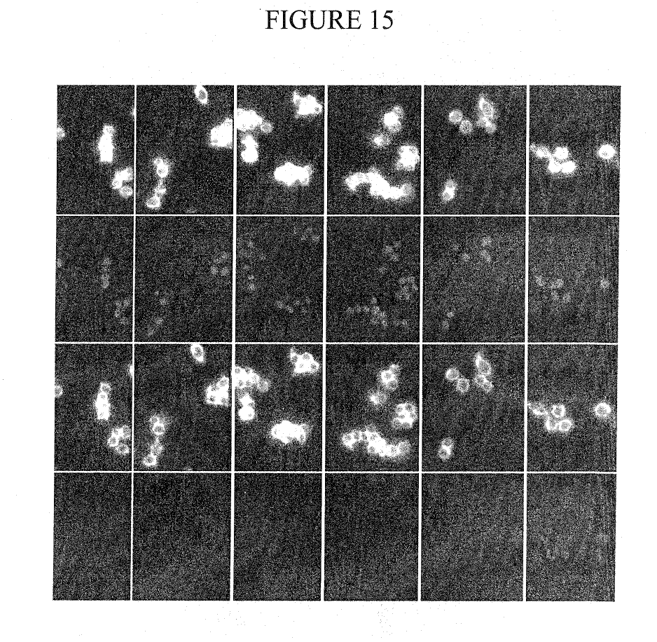

As seen in FIG. 15, protocells with about 21, about 42, about 84, about 169, and about 556 copies of CD47 avoided phagocytosis by macrophages. Protocells with only about 11 copies of CD47 per protocell were uptaken by the macrophages.

### Example 8—In Vivo Biodistribution of Protocells Conjugated to CD47

CD47 is attached to the lipid bilayer of protocells with varying diameters, such as between about 30 nm and 100 nm. The protocells are then IV-injected into BALB/c mice (about 200 mg/kg dose). As a control, protocells with a similar diameter but not conjugated to CD47 are also administered to BALB/c mice by IV injection. Protocells with larger diameters (such as between 100 nm and 300 nm), either conjugated to CD47 or not conjugated to CD47, are also tested by administering the protocells to BALB/c mice to compare the impact of protocell size on biodistribution.

Periodic blood samples are drawn from the mice and analyzed for silica content. Silica present in the blood indicates that the protocells remain in circulation. Thus, protocells uptaken by macrophages and removed from circulation result in rapid decrease of blood silica content. In contrast, protocells that remain in circulation and evade the macrophages demonstrate slower decline in blood silica content during the course of the study.

Sequences

ASVHFPP (Ala-Ser-Val-His-Phe-Pro-Pro)
SEQ ID NO: 1
TATFWFQ (Thr-Ala-Thr-Phe-Trp-Phe-Gln)
SEQ ID NO: 2
TSPVALL (Thr-Ser-Pro-Val-Ala-Leu-Leu)
SEQ ID NO: 3
IPLKVHP (Ile-Pro-Leu-Lys-Val-His-Pro)
SEQ ID NO: 4
WPRLTNM (Trp-Pro-Arg-Leu-Thr-Asn-Met)
SEQ ID NO: 5
H₂N-SFSIILTPILPL-COOH, SEQ ID NO: 6
H₂N-SFSIILTPILPLGGC-COOH, SEQ ID NO: 7
H₂N-SFSIILTPILPLEEEGGC-COOH, SEQ ID NO: 8
GNQSSNFGPMKGGNFGGRSSGPYGGGGQYFAKPRNQ-
GGYGGC-COOH, SEQ I.D NO: 9,
RRMKWKK, SEQ ID NO: 10
PKKKRKV, SEQ ID NO: 11
KR[PAATKKAGQA]KKKK, SEQ ID NO: 12
H₂N-GLFHAIAHFIHGGWHGLIHGWYGGC-COOH, SEQ ID. NO: 13
H₂N-RRRRRRRR-COOH, SEQ ID NO: 14
YLFSVHWPPLKA, SEQ ID NO: 15
HAIYPRH peptide, SEQ ID NO: 16
TPDWLFP, SEQ ID NO: 17
TGAILHP, SEQ ID NO: 18

1. A protocell comprising a nanoparticulate core surrounded by a lipid bilayer, wherein the lipid bilayer comprises a CD47 molecule or an active fragment thereof conjugated to the lipid bilayer.

2-3. (canceled)

4. The protocell of claim 1, wherein the CD47 molecule or an active fragment thereof is conjugated to the lipid bilayer via a linker.

5. (canceled)

6. The protocell of claim 4, wherein the CD47 molecule or an active fragment thereof is conjugated to the lipid bilayer via a heterobifunctional crosslinker.

7. The protocell of claim 4, wherein the linker is an amine-to-carboxylic acid crosslinker, crosslinker is formed using thyl(dimethylaminopropyl) carbodiimide and N-hydroxylsuflosuccinimide, is an amine-to-sulfhydryl crosslinker, comprises a maleimide reactive group and an N-hydroxysuccinimide ester reactive group or is SM-PEGₙ.

8-15. (canceled)

16. The protocell of claim 1, wherein the CD47 molecule or the active fragment thereof is conjugated to the lipid bilayer via chelation.

17-22. (canceled)

23. The protocell of claim 1, wherein the protocell comprises an effective number of copies of the CD47 molecule or the active fragment thereof to avoid macrophage phagocytosis or wherein the protocell comprises about 21 or more copies of the CD47 molecule or the active fragment thereof.

24. (canceled)

25. The protocell of claim 1, wherein the lipid bilayer further comprises a cell-targeting species.

26. (canceled)

27. The protocell of claim 1, wherein the lipid bilayer further comprises a fusogenic peptide.

28. The protocell of claim 1, wherein the protocell is loaded with a cargo.

29. The protocell of claim 28, wherein the cargo is a diagnostic agent.

30. The protocell of claim 28, wherein the cargo is a therapeutic agent.

31. The protocell of claim 28, wherein the cargo is a nucleic acid, a polypeptide, a drug, and an imaging agent.

32-41. (canceled)

42. The protocell of claim 1, wherein the core has a diameter of about 10 nm to about 250 nm.

43-45. (canceled)

46. The protocell of claim 1, wherein the lipid bilayer comprises a lipid selected from the group consisting of 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-dioleoyl-sn-glycero-3-[phosphor-L-serine] (DOPS), 1,2-dioleoyl-3-trimethylammonium-propane (18:1 DOTAP), 1,2-dioleoyl-sn-glycero-3-phospho-(1′-rac-glycerol) (DOPG), 1,2-dioleoyl-sn-glycero-3-phosphoethanolamine (DOPE), 1,2-dipalmitoyl-sn-glycero-3-phosphoethanolamine (DPPE), 1,2-dioleoyl-sn-glycero-3-phosphoethanolamine-N-[methoxy(polyethylene glycol)-2000] (18:1 PEG-2000 PE), 1,2-dipalmitoyl-sn-glycero-3-phosphoethanolamine-N-[methoxy(polyethylene glycol)-2000] (16:0 PEG-2000 PE), 1-Oleoyl-2-[12-[(7-nitro-2-1,3-benzoxadiazol-4-yl)amino]lauroyl]-sn-Glycero-3-Phosphocholine (18:1-12:0 NBD PC), 1-palmitoyl-2-{12-[(7-nitro-2-1,3-benzoxadiazol-4-yl)amino]lauroyl}-sn-glycero-3-phosphocholine (16:0-12:0 NBD PC), and cholesterol.

47-66. (canceled)

67. A method of treating cancer in a subject, comprising administering to the subject a pharmaceutical composition comprising the protocell of claim 1, wherein the protocells are loaded with an anticancer agent.

68. The method of claim 67, wherein the cancer is hepatocellular cancer.

69. A method of treating a viral infection in a subject, comprising administering to the subject the pharmaceutical composition of claim 1, wherein the protocells are loaded with an antiviral agent.

70. A method of delivering a cargo to a cell comprising contacting the protocell of claim 1 with a cell.

71. The method of claim 70, wherein the cell is a cancer cell.

72-74. (canceled)
